SEQUENCE LISTING (23 sequences)

SEQ ID NO: 1 - DNA, 1581 bp, Myceliophthora thermophila
atgtacgccaagttcgcgaccctcgccgcccttgtggctggcgccgctgctcagaacgcctgcactctgaccgctgagaaccacccctcgctgacgtggtccaagtgcacgtctggcggcagctgcaccagcgtccagggttccatcaccatcgacgccaactggcggtggactcaccggaccgatagcgccaccaactgctacgagggcaacaagtgggatacttcgtactgcagcgatggtccttcttgcgcctccaagtgctgcatcgacggcgctgactactcgagcacctatggcatcaccacgagcggtaactccctgaacctcaagttcgtcaccaagggccagtactcgaccaacatcggctcgcgtacctacctgatggagagcgacaccaagtaccagatgttccagctcctcggcaacgagttcaccttcgatgtcgacgtctccaacctcggctgcggcctcaatggcgccctctacttcgtgtccatggatgccgatggtggcatgtccaagtactcgggcaacaaggcaggtgccaagtacggtaccggctactgtgattctcagtgcccccgcgacctcaagttcatcaacggcgaggccaacgtagagaactggcagagctcgaccaacgatgccaacgccggcacgggcaagtacggcagctgctgctccgagatggacgtctgggaggccaacaacatggccgccgccttcactccccacccttgcaccgtgatcggccagtcgcgctgcgagggcgactcgtgcggcggtacctacagcaccgaccgctatgccggcatctgcgaccccgacggatgcgacttcaactcgtaccgccagggcaacaagaccttctacggcaagggcatgacggtcgacacgaccaagaagatcacggtcgtcacccagttcctcaagaactcggccggcgagctctccgagatcaagcggttctacgtccagaacggcaaggtcatccccaactccgagtccaccatcccgggcgtcgagggcaactccatcacccaggactggtgcgaccgccagaaggccgccttcggcgacgtgaccgacttccaggacaagggcggcatggtccagatgggcaaggccctcgcggggcccatggtcctcgtcatgtccatctgggacgaccacgccgtcaacatgctctggctcgactccacctggcccatcgacggcgccggcaagccgggcgccgagcgcggtgcctgccccaccacctcgggcgtccccgctgaggtcgaggccgaggcccccaactccaacgtcatcttctccaacatccgcttcggccccatcggctccaccgtctccggcctgcccgacggcggcagcggcaaccccaacccgcccgtcagctcgtccaccccggtcccctcctcgtccaccacatcctccggttcctccggcccgactggcggcacgggtgtcgctaagcactatgagcaatgcggaggaatcgggttcactggccctacccagtgcgagagcccctacacttgcaccaagctgaatgactggtactcgcagtgcctgtaa

SEQ ID NO: 2 - protein, 509 aa, Myceliophthora thermophila
QNACTLTAENHPSLTWSKCTSGGSCTSVQGSITIDANWRWTHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 3 - protein, 17 aa, Myceliophthora thermophila
MYAKFATLAALVAGAAA

SEQ ID NO: 4 - protein, 538 aa, thielavia australiensis
MYAKFATLAALVAGASAQAVCSLTAETHPSLTWQKCTAPGSCTNVAGSITIDANWRWTHQTSSATNCYSGSKWDSSICTTGTDCASKCCIDGAEYSSTYGITTSGNALNLKFVTKGQYSTNIGSRTYLMESDTKYQMFKLLGNEFTFDVDVSNLGCGLNGALYFVSMDADGGMSKYSGNKAGAKYGTGYCDAQCPRDLKFINGEANVEGWESSTNDANAGSGKYGSCCTEMDVWEANNMATAFTPHPCTTIGQTRCEGDTCGGTYSSDRYAGVCDPDGCDFNSYRQGNKTFYGKGMTVDTTKKITVVTQFLKNSAGELSEIKRFYAQDGKVIPNSESTIAGIPGNSITKAYCDAQKTVFQNTDDFTAKGGLVQMGKALAGDMVLVMSVWDDHAVNMLWLDSTYPTDQVGVAGAERGACPTTSGVPSDVEANAPNSNVIFSNIRFGPIGSTVQGLPSSGGTSSSSSAAPQSTSTKASTTTSAVRTTSTATTKTTSSAPAQGTNTAKHWQQCGGNGWTGPTVCESPYKCTKQNDWYSQCL

SEQ ID NO: 5 - protein, 525 aa, Humicola grisea
MRTAKFATLAALVASAAAQQACSLTTERHPSLSWKKCTAGGQCQTVQASITLDSNWRWTHQVSGSTNCYTGNKWDTSICTDAKSCAQNCCVDGADYTSTYGITTNGDSLSLKFVTKGQYSTNVGSRTYLMDGEDKYQTFELLGNEFTFDVDVSNIGCGLNGALYFVSMDADGGLSRYPGNKAGAKYGTGYCDAQCPRDIKFINGEANIEGWTGSTNDPNAGAGRYGTCCSEMDIWEANNMATAFTPHPCTIIGQSRCEGDSCGGTYSNERYAGVCDPDGCDFNSYRQGNKTFYGKGMTVDTTKKITVVTQFLKDANGDLGEIKRFYVQDGKIIPNSESTIPGVEGNSITQDWCDRQKVAFGDIDDFNRKGGMKQMGKALAGPMVLVMSIWDDHASNMLWLDSTFPVDAAGKPGAERGACPTTSGVPAEVEAEAPNSNVVFSNIRFGPIGSTVAGLPGAGNGGNNGGNPPPPTTTTSSAPATTTTASAGPKAGRWQQCGGIGFTGPTQCEEPYTCTKLNDWYSQCL

SEQ ID NO: 6 - protein, 532 aa, Chaetomium thermophilum
MMYKKFAALAALVAGASAQQACSLTAENHPSLTWKRCTSGGSCSTVNGAVTIDANWRWTHTVSGSTNCYTGNQWDTSLCTDGKSCAQTCCVDGADYSSTYGITTSGDSLNLKFVTKHQYGTNVGSRVYLMENDTKYQMFELLGNEFTFDVDVSNLGCGLNGALYFVSMDADGGMSKYSGNKAGAKYGTGYCDAQCPRDLKFINGEANVGNWTPSTNDANAGFGRYGSCCSEMDVWEANNMATAFTPHPCTTVGQSRCEADTCGGTYSSDRYAGVCDPDGCDFNAYRQGDKTFYGKGMTVDTNKKMTVVTQFHKNSAGVLSEIKRFYVQDGKIIANAESKIPGNPGNSITQEYCDAQKVAFSNTDDFNRKGGMAQMSKALAGPMVLVMSVWDDHYANMLWLDSTYPIDQAGAPGAERGACPTTSGVPAEIEAQVPNSNVIFSNIRFGPIGSTVPGLDGSNPGNPTTTVVPPASTSTSRPTSSTSSPVSTPTGQPGGCTTQKWGQCGGIGYTGCTNCVAGTTCTQLNPWYSQCL

SEQ ID NO: 7 - protein, 522 aa, Sordaria macrospora
MLAKFAALAALVASANAQAVCSLTAETHPSLNWSKCTSSGCTNVAGSVTIDANWRWTHTLSGSTNCYNGNKWDTTICSTNTDCATKCCVDGAEYSSTYGIQTSGNSLSLQFVTKGQYSTNIGSRTYLMNGNDAYQGFELLGNEFTFDVDVSGTGCGLNGALYFVSMDLDGGKSKYSTNKAGAKYGTGYCDAQCPRDLKYINGVGNVEGWSSSTNDPNAGIGNHGTCCSEMDIWEANKVSTAFTPHPCTTIDQHMCEGNSCGGTYSTDRYGGTCDADGCDFNSYRMGNTTFYGEGKTVDTRSKFTVVTQFIKNSAGDLGEIKRFYVQNGKVIENSQSNIAGVSGNSITQSFCDAQKTAFGDVGDFNVKGGLKQMGKALAKPMVLVMSIWDDHAANMLWLDSTYPVEGGSPGAYRGECPTTSGVPAEVEANAPNSKVIFSNIKFGPIGSTFSGGSSGGTPSNPSSSVKPVTSTAKPTTTSTASNPSGTGAAHWAQCGGIGFNGPTTCQSPYTCQKINDYYSQCL

SEQ ID NO: 8 - protein, 532 aa, Chaetomidium pingtungium
MLASTFSYRMYKTALILAALLGSGQAQQVGTSQAEVHPSMTWQSCTAGGSCTTNNGKVVIDANWRWVHKVGDYTNCYTGNTWDTTICPDDATCASNCALEGANYESTYGVTASGNSLRLNFVTTSQQKNIGSRLYMMKDDSTYEMFKLLNQEFTFDVDVSNLPCGLNGALYFVAMDAGGGMSKYPTNKAGAKYGTGYCDSQCPRDLKFINGQANVEGWQPSSNDANAGTGNHGSCCAEMDIWEANSISTAFTPHPCDTPGQVMCTGDACGGTYSSDRYGGTCDPDGCDFNSFRQGNKTFYGPGMTVDTKSKFTVVTQFITDDGTSSGTLKEIKRFYVQNGKVIPNSESTWTGVSGNSITTEYCTAQKSLFQDQNVFEKHGGLEGMGAALAQGMVLVMSLWDDHSANMLWLDSNYPTTASSTTPGVARGTCDISSGVPADVEANHPDAYVVYSNIKVGPIGSTFNSGGSNPGGGTTTTTTTQPTTTTTTAGNPGGTGVAQHYGQCGGIGWTGPTTCASPYTCQKLNDYYSQCL

SEQ ID NO: 9 - protein, 454 aa, Botryosphaeria rhodina
MLTQAVLATLATLAASQQVGTQKEEVHPSMTWQTCTSSGCTTNQGSIVVDANWRWVHNTEGYTNCYTGNTWNADYCTDNTECASNCALDGADYSGTYGATTSGDSLRLNFITNGQQKNIGSRMYLMQDDETYAVHKLLNKEFTFDVDTSKLPCGLNGAVYFVSMDADGGMAKFPDNKAGAKYGTGYCDSQCPRDLKFIDGKANVEGWVPSENDSNAGVGNLGSCCAEMDIWEANSISTAYTPHSCKTVAQHSCTGDDCGGTYSATRYAGDCDPDGCDFNSYRQGVKDFYGPGMTVDSNSVVTVVTQFITNDGTASGTLSEIKRFYVQNGKVIPNSESTIAGVSGNSITSAYCDAQKEVFGDNTSFQDQGGLASMSQALNAGMVLVMSIWDDHHSNMLWLDSDYPVDADPSQPGISRGTCPTTSGVPSEVEESAASAYVVYSNIKVGDLNSTFSA

SEQ ID NO: 10 - protein, 458 aa, Trichophaea saccata
MQRLLVLLTSLLAFTYGQQVGTQQAEVHPSMTWQQCTKSGGCTTKNGKVVIDANWRWVHNVGGYTNCYTGNTWDSSLCPDDVTCAKNCALDGADYSGTYGVTAGGNSLKLTFVTKGQYSTNVGSRLYMLADDSTYQMYNLLNQEFTFDVDVSNLPCGLNGALYFVSMDKDGGMSKYSGNKAGAKYGTGYCDSQCPRDLKFINGQGNVEGWKPSSNDANAGVGGHGSCCAEMDVWEANSISAAVTPHSCSTTSQTMCNGDSCGGTYSATRYAGVCDPDGCDFNSYRMGDTTFYGKGKTVDTSSKFTVVTQFITDTGTASGSLTEIRRFYVQNGKLIPNSQSKISGVTGNSITSAFCDAQKAAFGDNYTFKDKGGFASMTTAMKNGMVLVMSLWDDHYANMLWLDSDYPTNADSSKPGVARGTCPTSSGVPSDVETNNASASVTYSNIRFGDLNSTYTAQ

SEQ ID NO: 11 - protein, 526 aa, Aspergillus nidulans
MASSFQLYKALLFFSSLLSAVQAQKVGTQQAEVHPGLTWQTCTSSGSCTTVNGEVTIDANWRWLHTVNGYTNCYTGNEWDTSICTSNEVCAEQCAVDGANYASTYGITTSGSSLRLNFVTQSQQKNIGSRVYLMDDEDTYTMFYLLNKEFTFDVDVSELPCGLNGAVYFVSMDADGGKSRYATNEAGAKYGTGYCDSQCPRDLKFINGVANVEGWESSDTNPNGGVGNHGSCCAEMDIWEANSISTAFTPHPCDTPGQTLCTGDSCGGTYSNDRYGGTCDPDGCDFNSYRQGNKTFYGPGLTVDTNSPVTVVTQFLTDDNTDTGTLSEIKRFYVQNGVVIPNSESTYPANPGNSITTEFCESQKELFGDVDVFSAHGGMAGMGAALEQGMVLVLSLWDDNYSNMLWLDSNYPTDADPTQPGIARGTCPTDSGVPSEVEAQYPNAYVVYSNIKFGPIGSTFGNGGGSGPTTTVTTSTATSTTSSATSTATGQAQHWEQCGGNGWTGPTVCASPWACTVVNSWYSQCL

SEQ ID NO: 12 - protein, 455 aa, Schizophyllum commune
MFPAAALVSLTLAAIARAQQVGNLTAETHPSLSWQECTSSGCTDHSAAITIDANWRWLHTVEGYDNCYTGNEWDESVCTDGATCASSCALDGADYSGTYGITTSGDALSLKFVTSGQQKNIGSRTYLMNGEDKYQMFKLKNKEFTFTVDMSTLPCGLNGALYFVEMDEDGGVSKYPTNKAGAKYGTGYCDSQCPHDIKFINGEANSEGWAGSDNDPNAGTGTYGSCCSEMDIWEANSQAAAYTPHVCTVEGQTRCEGTDCGDDDDRYSGVCDKDGCDFNSYRMGDTTFLGEGLTIDTTQPITVVTQFITADNTTSGALSEIRRLYVQGGEVVANSATKIEGLDDYDSITDQFCADQKEVFGDQNYFGTLGGLEAMGESLDRGHVLVMSIWDDHAVNMLWLDSDYPLDKDPSEPGVSRGPCGTDSGVPTDVEAQSPDATVIYSAIKFGDIGTTYSA

SEQ ID NO: 13 - protein, 506 aa, Agaricus bisporus
MFPRSILLALSLTAVALGQQVGTNMAENHPSLTWQRCTSSGCQNVNGKVTLDANWRWTHRINDFTNCYTGNEWDTSICPDGVTCAENCALDGADYAGTYGVTSSGTALTLKFVTESQQKNIGSRLYLMADDSNYEIFNLLNKEFTFDVDVSKLPCGLNGALYFSEMAADGGMSSTNTAGAKYGTGYCDSQCPRDIKFIDGEANSEGWEGSPNDVNAGTGNFGACCGEMDIWEANSISSAYTPHPCREPGLQRCEGNTCSVNDRYATECDPDGCDFNSFRMGDKSFYGPGMTVDTNQPITVVTQFITDNGSDNGNLQEIRRIYVQNGQVIQNSNVNIPGIDSGNSISAEFCDQAKEAFGDERSFQDRGGLSGMGSALDRGMVLVLSIWDDHAVNMLWLDSDYPLDASPSQPGISRGTCSRDSGKPEDVEANAGGVQVVYSNIKFGDINSTFNNNGGGGGNPSPTTTRPNSPAQTMWGQCGGQGWTGPTACQSPSTCHVINDFYSQCF

SEQ ID NO: 14 - DNA, 2487 bp, Myceliophthora thermophila
atgaggacctcctctcgtttaatcggtgcccttgcggcggcactcttgccgtctgcccttgcgcagaacaacgcgccggtaaccttcaccgacccggactcgggcattaccttcaacacgtggggtctcgccgaggattctccccagactaagggcggtttcacttttggtgttgctctgccctctgatgccctcacgacagacgccaaggagttcatcggttacttgaaatgcgcgaggaacgatgagagcggttggtgcggtgtctccctgggcggccccatgaccaactcgctcctcatcgcggcctggccccacgaggacaccgtctacacctctctccgcttcgccaccggctatgccatgccggatgtctaccagggggacgccgagatcacccaggtctcctcctctgtcaactcgacgcacttcagcctcatcttcaggtgcgagaactgcctgcaatggagtcaaagcggcgccaccggcggtgcctccacctcgaacggcgtgttggtcctcggctgggtccaggcattcgccgaccccggcaacccgacctgccccgaccagatcaccctcgagcagcacgacaacggcatgggtatctggggtgcccagctcaactccgacgccgccagcccgtcctacaccgagtgggccgcccaggccaccaagaccgtcacgggtgactgcggcggtcccaccgagacctctgtcgtcggtgtccccgttccgacgggcgtctcgttcgattacatcgtcgtgggcggcggtgccggtggcatccccgccgccgacaagctcagcgaggccggcaagagtgtgctgctcatcgagaagggctttgcctcgaccgccaacaccggaggcactctcggccccgagtggctcgagggccacgaccttacccgctttgacgtgccgggtctgtgcaaccagatctgggttgactccaaggggatcgcttgcgaggataccgaccagatggctggctgtgtcctcggcggcggtaccgccgtgaatgccggcctgtggttcaagccctactcgctcgactgggactacctcttccctagtggttggaagtacaaagacgtccagccggccatcaaccgcgccctctcgcgcatcccgggcaccgatgctccctcgaccgacggcaagcgctactaccaacagggcttcgacgtcctctccaagggcctggccggcggcggctggacctcggtcacggccaataacgcgccagacaagaagaaccgcaccttctcccatgcccccttcatgttcgccggcggcgagcgcaacggcccgctgggcacctacttccagaccgccaagaagcgcagcaacttcaagctctggctcaacacgtcggtcaagcgcgtcatccgccagggcggccacatcaccggcgtcgaggtcgagccgttccgcgacggcggttaccaaggcatcgtccccgtcaccaaggttacgggccgcgtcatcctctctgccggtacctttggcagtgcaaagatcctgctgaggagcggtatcggtccgaacgatcagctgcaggttgtcgcggcctcggagaaggatggccctaccatgatcagcaactcgtcctggatcaacctgcctgtcggctacaacctggatgaccacctcaacaccgacactgtcatctcccaccccgacgtcgtgttctacgacttctacgaggcgtgggacaatcccatccagtctgacaaggacagctacctcaactcgcgcacgggcatcctcgcccaagccgctcccaacattgggcctatgttctgggaagagatcaagggtgcggacggcattgttcgccagctccagtggactgcccgtgtcgagggcagcctgggtgcccccaacggcaagaccatgaccatgtcgcagtacctcggtcgtggtgccacctcgcgcggccgcatgaccatcaccccgtccctgacaactgtcgtctcggacgtgccctacctcaaggaccccaacgacaaggaggccgtcatccagggcatcatcaacctgcagaacgccctcaagaacgtcgccaacctgacctggctcttccccaactcgaccatcacgccgcgccaatacgttgacagcatggtcgtctccccgagcaaccggcgctccaaccactggatgggcaccaacaagatcggcaccgacgacgggcgcaagggcggctccgccgtcgtcgacctcaacaccaaggtctacggcaccgacaacctcttcgtcatcgacgcctccatcttccccggcgtgcccaccaccaaccccacctcgtacatcgtgacggcgtcggagcacgcctcggcccgcatcctcgccctgcccgacctcacgcccgtccccaagtacgggcagtgcggcggccgcgaatggagcggcagcttcgtctgcgccgacggctccacgtgccagatgcagaacgagtggtactcgcagtgcttgtga

SEQ ID NO: 15 - protein, 828 aa, Myceliophthora thermophila
MRTSSRLIGALAAALLPSALAQNNAPVTFTDPDSGITFNTWGLAEDSPQTKGGFTFGVALPSDALTTDAKEFIGYLKCARNDESGWCGVSLGGPMTNSLLIAAWPHEDTVYTSLRFATGYAMPDVYQGDAEITQVSSSVNSTHFSLIFRCENCLQWSQSGATGGASTSNGVLVLGWVQAFADPGNPTCPDQITLEQHDNGMGIWGAQLNSDAASPSYTEWAAQATKTVTGDCGGPTETSVVGVPVPTGVSFDYIVVGGGAGGIPAADKLSEAGKSVLLIEKGFASTANTGGTLGPEWLEGHDLTRFDVPGLCNQIWVDSKGIACEDTDQMAGCVLGGGTAVNAGLWFKPYSLDWDYLFPSGWKYKDVQPAINRALSRIPGTDAPSTDGKRYYQQGFDVLSKGLAGGGWTSVTANNAPDKKNRTFSHAPFMFAGGERNGPLGTYFQTAKKRSNFKLWLNTSVKRVIRQGGHITGVEVEPFRDGGYQGIVPVTKVTGRVILSAGTFGSAKILLRSGIGPNDQLQVVAASEKDGPTMISNSSWINLPVGYNLDDHLNTDTVISHPDVVFYDFYEAWDNPIQSDKDSYLNSRTGILAQAAPNIGPMFWEEIKGADGIVRQLQWTARVEGSLGAPNGKTMTMSQYLGRGATSRGRMTITPSLTTVVSDVPYLKDPNDKEAVIQGIINLQNALKNVANLTWLFPNSTITPRQYVDSMVVSPSNRRSNHWMGTNKIGTDDGRKGGSAVVDLNTKVYGTDNLFVIDASIFPGVPTTNPTSYIVTASEHASARILALPDLTPVPKYGQCGGREWSGSFVCADGSTCQMQNEWYSQCL

SEQ ID NO: 16 - DNA, 2364 bp, Myceliophthora thermophila
atgaagctactcagccgcgttggggcgaccgccctagcggcgacgttgtcactgcagcaatgtgcagcccagatgaccgaggggacctacaccgatgaggctaccggtatccaattcaagacgtggaccgcctccgagggcgcccctttcacgtttggcttgaccctccccgcggacgcgctggaaaaggatgccaccgagtacattggtctcctgcgttgccaaatcaccgatcccgcctcgcccagctggtgcggtatctcccacggccagtccggccagatgacgcaggcgctgctgctggtcgcctgggccagcgaggacaccgtctacacgtcgttccgctacgccaccggctacacgctccccggcctctacacgggcgacgccaagctgacccagatctcctcctcggtcagcgaggacagcttcgaggtgctgttccgctgcgaaaactgcttctcctgggaccaggatggcaccaagggcaacgtctcgaccagcaacggcaacctggtcctcggccgcgccgccgcgaaggatggtgtgacgggccccacgtgcccggacacggccgagttcggtttccatgataacggtttcggacagtggggtgccgtgcttgagggtgctacttcggactcgtacgaggagtgggctaagctggccacgaccacgcccgagaccacctgcgatggcactggccccggcgacaaggagtgcgttccggctcccgaggacacgtatgattacatcgttgtcggtgccggcgccggtggtatcaccgtcgccgacaagctcagcgaggccggccacaaggtccttctcatcgagaagggacccccttcgaccggcctgtggaacgggaccatgaagcccgagtggctcgagagcaccgaccttacccgcttcgacgttcccggcctgtgcaaccagatctgggtcgactctgccggcatcgcctgcaccgataccgaccagatggcgggctgcgttctcggcggtggcaccgctgtcaacgctggtttgtggtggaagccccaccccgctgactgggatgagaacttccccgaagggtggaagtcgagcgatctcgcggatgcgaccgagcgtgtcttcaagcgcatccccggcacgtcgcacccgtcgcaggacggcaagttgtaccgccaggagggcttcgaggtcatcagcaagggcctggccaacgccggctggaaggaaatcagcgccaacgaggcgcccagcgagaagaaccacacctatgcacacaccgagttcatgttctcgggcggtgagcgtggcggccccctggcgacgtaccttgcctcggctgccgagcgcagcaacttcaacctgtggctcaacactgccgtccggagggccgtccgcagcggcagcaaggtcaccggcgtcgagctcgagtgcctcacggacggtggcttcagcgggaccgtcaacctgaatgagggcggtggtgtcatcttctcggccggcgctttcggctcggccaagctgctccttcgcagcggtatcggtcctgaggaccagctcgagattgtggcgagctccaaggacggcgagaccttcactcccaaggacgagtggatcaacctccccgtcggccacaacctgatcgaccatctcaacactgacctcattatcacgcacccggatgtcgttttctatgacttctatgcggcctgggacgagcccatcacggaggataaggaggcctacctgaactcgcggtccggcattctcgcccaggcggcgcccaatatcggccctatgatgtgggatcaagtcacgccgtccgacggcatcacccgccagttccagtggacatgccgtgttgagggcgacagctccaagaccaactcgacccacgccatgaccctcagccagtacctcggccgtggcgtcgtctcgcgcggccggatgggcatcacctccgggctgagcacgacggtggccgagcacccgtacctgcacaacaacggcgacctggaggcggtcatccaggggatccagaacgtggtggacgcgctcagccaggtggccgacctcgagtgggtgctcccgccgcccgacgggacggtggccgactacgtcaacagcctgatcgtctcgccggccaaccgccgggccaaccactggatgggcacggccaagctgggcaccgacgacggccgctcgggcggcacctcggtcgtcgacctcgacaccaaggtgtacggcaccgacaacctgttcgtcgtcgacgcgtccgtcttccccggcatgtcgacgggcaacccgtcggccatgatcgtcatcgtggccgagcaggcggcgcagcgcatcctggccctgcggtcttaa

SEQ ID NO: 17 - protein, 787 aa, Myceliophthora thermophila
MKLLSRVGATALAATLSLQQCAAQMTEGTYTDEATGIQFKTWTASEGAPFTFGLTLPADALEKDATEYIGLLRCQITDPASPSWCGISHGQSGQMTQALLLVAWASEDTVYTSFRYATGYTLPGLYTGDAKLTQISSSVSEDSFEVLFRCENCFSWDQDGTKGNVSTSNGNLVLGRAAAKDGVTGPTCPDTAEFGFHDNGFGQWGAVLEGATSDSYEEWAKLATTTPETTCDGTGPGDKECVPAPEDTYDYIVVGAGAGGITVADKLSEAGHKVLLIEKGPPSTGLWNGTMKPEWLESTDLTRFDVPGLCNQIWVDSAGIACTDTDQMAGCVLGGGTAVNAGLWWKPHPADWDENFPEGWKSSDLADATERVFKRIPGTSHPSQDGKLYRQEGFEVISKGLANAGWKEISANEAPSEKNHTYAHTEFMFSGGERGGPLATYLASAAERSNFNLWLNTAVRRAVRSGSKVTGVELECLTDGGFSGTVNLNEGGGVIFSAGAFGSAKLLLRSGIGPEDQLEIVASSKDGETFTPKDEWINLPVGHNLIDHLNTDLIITHPDVVFYDFYAAWDEPITEDKEAYLNSRSGILAQAAPNIGPMMWDQVTPSDGITRQFQWTCRVEGDSSKTNSTHAMTLSQYLGRGVVSRGRMGITSGLSTTVAEHPYLHNNGDLEAVIQGIQNVVDALSQVADLEWVLPPPDGTVADYVNSLIVSPANRRANHWMGTAKLGTDDGRSGGTSVVDLDTKVYGTDNLFVVDASVFPGMSTGNPSAMIVIVAEQAAQRILALRS

SEQ ID NO: 18 - protein, 526 aa, Artificial Sequence
  Synthetic polypeptide for M. thermophila CBH1a variant 24
MYAKFATLAALVAGAAAQNACTLTAENHPSLTWSKCTSGGSCTSVQGSITIDANWRWTHRTDSATNCYEGNKWDTSWCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 19 - protein, 526 aa, Artificial Sequence
  Synthetic polypeptide for M. thermophila CBH1a variant 207
MYAKFATLAALVAGAAAQNACTLNAENHPSLTWSKCTSGGSCTSVQGSITIDANWRWTHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALEGPMVLVMSIWDDHADNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 20 - protein, 526 aa, Artificial Sequence
  Synthetic polypeptide for M. thermophila CBH1a variant 208
MYAKFATLAALVAGAAAQNACTLNAENHPSLTWSKCTSGGSCTSVQGSITIDANWRWTHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQEYCDRQKAAFGDVTDFQDKGGMVQMGKALAGPMVLVMSIWDDHADNMLWLDSTWPIDGAGKPGAERGACPTTSGVPAEVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 21 - protein, 526 aa, Artificial Sequence
  Synthetic polypeptide for M. thermophila CBH1a variant 251
MYAKFATLAALVAGAAAQNACTLTAENHPSLTWSKCTSGGSCTSVQGSITIDANWRWTHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGEGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQDWCDRQKAAFGDVTDFQDKGGMVQMGKALAGPMVLVMSIWDDHAVNMLWLDSTWPIDGAGKPGAERGACPTTSGVPANVEAEAPNSNVIFSNIRFGPIGSTVSGLPDGGSGNPNPPVSSSTPVPSSSTTSSGSSGPTGGTGVAKHYEQCGGIGFTGPTQCESPYTCTKLNDWYSQCL

SEQ ID NO: 22 - protein, 596 aa, Artificial Sequence
  Synthetic polypeptide for M. thermophila CBH1a variant 472
MYAKFATLAALVAGAAAQNACTLTAENHPSLTWSKCTSGGSCTSVQGSITIDANWRWTHRTDSATNCYEGMYAKFATLAALVAGAAAQNACTLNAENHPSLTWSKCTSGGSCTSVQGSITIDANWRWVHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQEYCDRQKAAFGDVDDFQDKGGMVQMGKALAGPMVLVMSIWDDHADNMLWLDSTWPIDGAGKPGAERGACPTTSGVPAEVEAEAPNSNVIFSNIRFGPIGSTYSGLPDGGSGNPNPPVSSSTPVPSSSTTSSGSSGPTGGTGVAKHYEQCGGQGFTGPTQCESPYTCTKLNDWYSQCL

SEQ ID NO: 23 - protein, 526 aa, Artificial Sequence
  Synthetic polypeptide for M. thermophila CBH1a variant 473
MYAKFATLAALVAGAAAQNACTLTAENHPSLTWSKCTSGGSCTSVQGSITIDANWRWVHRTDSATNCYEGNKWDTSYCSDGPSCASKCCIDGADYSSTYGITTSGNSLNLKFVTKGQYSTNIGSRTYLMESDTKYQMFQLLGNEFTFDVDVSNLGCGLNGALYFVSMDADGGMSKYSGNKAGAKYGTGYCDSQCPRDLKFINGEANVENWQSSTNDANAGTGKYGSCCSEMDVWEANNMAAAFTPHPCTVIGQSRCEGDSCGGTYSTDRYAGICDPDGCDFNSYRQGNKTFYGKGMTVDTTKKITVVTQFLKNSAGELSEIKRFYVQNGKVIPNSESTIPGVEGNSITQEYCDRQKAAFGDVDDFQDKGGMVQMGKALAGPMVLVMSIWDDHADNMLWLDSTWPIDGAGKPGAERGACPTTSGVPAEVEAEAPNSNVIFSNIRFGPIGSTYSGLPDGGSGNPNPPVSSSTPVPSSSTTSSGSSGPTGGTGVAKHYEQCGGQGFTGPTQCESPYTCTKLNDWYSQCL

(19) United States
(12) Patent Application Publication
(10) Pub. No.: US 2015/0087028 A1
(43) Pub. Date: Mar. 26, 2015

(54) CBH1A VARIANTS

(71) Applicant: Codexis, Inc., Redwood City, CA (US)

(72) Inventors: Behnaz Behrouzian, Sunnyvale, CA (US); Xinkai Xie, Castro Valley, CA (US); Kui Chan, Belmont, CA (US); Xiyun Zhang, Fremont, CA (US); Vesna Mitchell, San Jose, CA (US); Douglas A. Hattendorf, Mountain View, CA (US)

(21) Appl. No.: 14/384,133

(22) PCT Filed: Mar. 12, 2013
(86) PCT No.: PCT/US2013/030558

§ 371 Date: Sep. 9, 2014

Related U.S. Application Data
(60) Provisional application No. 61/609,843, filed on Mar. 12, 2012.

(51) Int. Cl. C12P 19/14; C12P 19/02; C12P 7/20; C12P 13/00; C12P 7/40; C12P 7/18; C12N 9/42; C12P 7/04

(52) U.S. Cl. CPC C12P 19/14; C12N 9/2437; C12P 19/02; C12P 7/04; C12P 13/001; C12P 7/40; C12P 7/18; C12P 7/20
USPC 435/99; 536/23.2; 435/209; 435/320.1; 435/157; 435/128; 435/136; 435/158; 435/159; 435/254.11; 435/254.3; 435/254.7; 435/254.8; 435/254.4; 435/254.5; 435/254.9; 435/254.6; 435/254.2; 435/254.22; 435/254.21; 435/254.23; 435/252.3; 435/252.31; 435/252.32; 435/252.33; 435/252.34; 435/252.35; 435/252.2

(57) ABSTRACT

The invention relates to recombinant expression of variant forms of M. thermophila CBH1a and homologs thereof, having improved thermoactivity, specific activity, and other desirable properties. Also provided are methods for producing ethanol and other valuable organic compounds by combining cellobiohydrolase variants with cellulosic materials.

### CROSS-REFERENCES TO RELATED APPLICATIONS

This application claims benefit of priority of U.S. Provisional Application No. 61/609,843, filed Mar. 12, 2012, the entire content of which is incorporated herein by reference.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 90834-832291_ST25.TXT, created on Mar. 6, 2013, 99,750 bytes, machine format IBM-PC, MS-Windows operating system, is hereby incorporated by reference.

### FIELD OF THE INVENTION

The invention relates to expression of recombinant cellobiohydrolase variants and their use in the production of fermentable sugars from cellulosic biomass.

### BACKGROUND OF THE INVENTION

Cellulosic biomass is a significant renewable resource for the generation of fermentable sugars. These sugars can be used as reactants in various metabolic processes, including fermentation, to produce biofuels, chemical compounds, and other commercially valuable end-products. While the fermentation of simple sugars such as glucose to ethanol is relatively straightforward, the efficient conversion of cellulosic biomass to fermentable sugars is challenging. See, e.g., Ladisch et al., 1983, Enzyme Microb. Technol. 5:82. Cellulose may be pretreated chemically, mechanically, enzymatically or in other ways to increase the susceptibility of cellulose to hydrolysis. Such pretreatment may be followed by the enzymatic conversion of cellulose to cellobiose, cello-oligosaccharides, glucose, and other sugars and sugar polymers, using enzymes that break down the β-1-4 glycosidic bonds of cellulose. These enzymes are collectively referred to as “cellulases.”

Cellulases are divided into three sub-categories of enzymes: 1,4-β-D-glucan glucanohydrolase (“endoglucanase” or “EG”); 1,4-β-D-glucan cellobiohydrolase (“exoglucanase”, “cellobiohydrolase”, or “CBH”); and β-D-glucoside-glucohydrolase (“β-glucosidase”, “cellobiase” or “BGL”). Endoglucanases break internal bonds and disrupt the crystalline structure of cellulose, exposing individual cellulose polysaccharide chains (“glucans”). Cellobiohydrolases incrementally shorten the glucan molecules, releasing mainly cellobiose units (a water-soluble β-1,4-linked dimer of glucose) as well as glucose, cellotriose, and cellotetrose. There are two main types of cellobiohydrolases: Type 1 cellobiohydrolases are members of the Glycoside Hydrolase Family 7, and cleave processively from the reducing end of cellulose chains. Type 2 cellobiohydrolases cleave processively from the non-reducing end of cellulose. β-glucosidases split cellobiose into glucose monomers.

Cellulases with improved properties for use in processing cellulosic biomass would reduce costs and increase the efficiency of production of biofuels and other commercially valuable compounds.

### BRIEF SUMMARY OF THE INVENTION

In one aspect, the present invention provides recombinant cellobiohydrolase variants that exhibit improved properties. In some embodiments, the cellobiohydrolase variants are superior to naturally occurring cellobiohydrolases under conditions required for saccharification of cellulosic biomass.

In some embodiments, a recombinant cellobiohydrolase variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises a mutation (e.g., an amino acid substitution) at one or more positions selected from 2, 23, 24, 25, 30, 39, 46, 58, 60, 61, 62, 63, 64, 75, 76, 77, 83, 96, 97, 111, 115, 117, 118, 119, 177, 178, 208, 216, 217, 221, 222, 223, 250, 251, 267, 268, 271, 275, 289, 294, 339, 343, 349, 350, 351, 354, 357, 362, 363, 367, 379, 392, 394, 403, 408, 409, 417, 424, 426, 427, 428, 433, 434, 451, 452, 453, 455, 456, 457, 458, 459, 464, 465, 466, 467, 469, 470, 472, 473, 474, 475, 476, 477, 478, 479, 480, 481, 482, 483, 484, 485, 486, 487, 489, 490, 491, 492, 494, 495, 496, 500, 501, 502, 504, 505, 506, 507, 508, 510, 511, 512, 513, 516, 517, 518, 519, 521, 523, 524, and/or 526, wherein the positions are numbered with reference to a wild-type M. thermophila CBH1a protein (e.g., SEQ ID NO:2).

In some embodiments, a recombinant cellobiohydrolase variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises a mutation (e.g., an amino acid substitution) at one or more positions selected from Y2, L23, T24, A25, S30, G39, Q46, T58, R60, T61, S62, S63, A64, T75, S76, Y77, S83, S96, S97, K111, K115, Q117, Y118, S119, S177, G178, E208, D216, A217, T221, G222, K223, V250, I251, T267, D268, A271, D275, K289, K294, I339, E343, Q349, D350, W351, R354, A357, V362, T363, D367, A379, H392, V394, W403, A408, G409, A417, V424, A426, E427, V428, P433, N434, V451, S452, G453, P455, D456, G457, G458, S459, P464, P465, V466, S467, S469, T470, V472, P473, S474, S475, S476, T477, T478, S479, S480, G481, S482, S483, G484, P485, T486, G487, T489, G490, V491, A492, H494, Y495, E496, G500, I501, G502, T504, G505, P506, T507, Q508, T510, S511, P512, Y513, T516, K517, L518, N519, W521, S523, Q524, and/or L526, wherein the positions are numbered with reference to a wild-type M. thermophila CBH1a protein (e.g., SEQ ID NO:2).

In some embodiments, a recombinant cellobiohydrolase variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises a mutation (e.g., an amino acid substitution) at one or more positions selected from Y2H, L23E, T24L, T24N, T24V, A25D, S30T, G39D, Q46H, T58Y, R60T, T61N, S62L, S63A, S63D, A64P, T75L, S76V, Y77W, S83T, S83W, S96E, S96N, S97G, K111Q, K115N, Y118D, Q117D, Q117E, Y118D, S119D, S177E, G178S, E208G, D216N, A217E, T221E, G222S, K223A, K223L, K223G, K223W, V2501, I251W, T267E, T267N, D268Y, A271V, D275N, K289E, K289W, K289R, K289D, K294Q, I339V, E343D, Q349T, D350A, D350E, W351Y, R354L, A357V, V362A, V362P, V362T, T363N, D367Y, A379E, H392A, H392E, H392P, H392S, V394A, V394D, W403Y, A408D, A408S, G409D, A417S, V424R, A426S, E427N, V428L, P433S, N434E, V451Y, S452E, G453L, P455F, P455L, P455Q, P455W, D456R, G457R, G458A, S459E, P464R, P465A, P465M, P465R, V466A, V466W, S467R, S469C, T470D, T470E, V472A, P473L, P473T, P473W, S474D, S475L, S475F, S475R, S475W, S476P, T477V, T478K, T478W, S479K, S480M, G481F, G481P, G481R, G481W, S482G, S483G, G484L, G484P, P485W, T486A, T486K, G487D, G4871, T489Y, G490A, G490W, V491A, V491P, A492C, A492V, H494A, H494R, H494E, H494K, H494W, Y495G, E496L, E496R, E496W, G500E, G500R, G500V, 1501 Q, 1501 S, 1501 W, G5021, G502L, G502Q, T504A, T504G, T504M, G505E, G505P, P506A, P506H, P506L, P506V, T507W, Q508W, T510R, S511P, S511W, P512A, P512R, Y513G, Y513W, T516A, T516K, T516R, K517E, K517G, K517P, L518A, L518E, N519H, W521A, S523A, S523W, Q524G, Q524P, Q524W, L526E, L526F, and/or L526P, wherein the positions are numbered with reference to a wild-type M. thermophila CBH1a protein (e.g., SEQ ID NO:2).

In some embodiments, a recombinant cellobiohydrolase variant is encoded by a polynucleotide that hybridizes under high stringency conditions to the complement of SEQ ID NO:1 and comprises one or more mutations (e.g., amino acid substitutions) as described herein.

In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises mutations at one or more positions (e.g., one, two, three, four, five, six or more positions) selected from 351, 363, 58, 451, 501, 394, 350, and 24, wherein the positions are numbered with reference to a wild-type M. thermophila CBH1a protein (e.g., SEQ ID NO:2). In some embodiments, the amino acid residue at position 351 is tryptophan (W351), the amino acid residue at position 363 is threonine (T363), the amino acid residue at position 58 is threonine (T58), the amino acid residue at position 451 is valine (V451), the amino acid residue at position 501 is isoleucine (I501), the amino acid residue at position 394 is valine (V394), the amino acid residue at position 350 is aspartic acid (D350), and the amino acid residue at position 24 is threonine (T24), and the cellobiohydrolase variant comprises mutations at one or more positions (e.g., one, two, three, four, five, six or more positions) selected from W351, T363, T58, V451, I501, V394, D350, and T24). In some embodiments, the variant comprises one or more amino acid substitutions (e.g., one, two, three, four, five, six or more amino acid substitutions) selected from W351Y, T363D, T58V, V451Y, I501Q, V394D, D350E, and T24N/V.

In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises mutations at positions 24 and 379, wherein the positions are numbered with reference to a wild-type M. thermophila CBH1a protein (e.g., SEQ ID NO:2). In some embodiments, the amino acid residue at position 24 is threonine (T24) and the amino acid residue at position 379 is alanine (A379), and the cellobiohydrolase variant comprises mutations at positions T24 and A379. In some embodiments, the variant comprises mutations at positions T24N/V and A379E.

In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises mutations at positions 24, 379, and 394, wherein the positions are numbered with reference to a wild-type M. thermophila CBH1a protein (e.g., SEQ ID NO:2). In some embodiments, the amino acid residue at position 24 is threonine (T24), the amino acid residue at position 379 is alanine (A379), and the amino acid residue at position 394 is valine (V394), and the cellobiohydrolase variant comprises mutations at positions T24, A379, and V394. In some embodiments, the variant comprises mutations at positions T24N, A379E, and V394D.

In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises mutations at positions 24, 350, 351, and 394, wherein the positions are numbered with reference to a wild-type M. thermophila CBH1a protein (e.g., SEQ ID NO:2). In some embodiments, the amino acid residue at position 24 is threonine (T24), the amino acid residue at position 350 is aspartic acid (D350), the amino acid residue at position 351 is tryptophan (W351), and the amino acid residue at position 394 is valine (V394), and the cellobiohydrolase variant comprises mutations at positions T24, D350, W351, and V394. In some embodiments, the variant comprises mutations at positions T24N, D350E, W351Y, and V394D.

In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises a mutation at position 77, wherein the positions are numbered with reference to a wild-type M. thermophila CBH1a protein (e.g., SEQ ID NO:2). In some embodiments, the amino acid residue at position 77 is tyrosine (Y77), and the cellobiohydrolase variant comprises a mutation at position Y77. In some embodiments, the variant comprises a mutation at position Y77W.

In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises mutations at positions 24, 58, 350, 351, 363, 394, 451, and 501, wherein the positions are numbered with reference to a wild-type M. thermophila CBH1a protein (e.g., SEQ ID NO:2). In some embodiments, the amino acid residue at position 24 is threonine (T24), the amino acid residue at position 58 is threonine (T58), the amino acid residue at position 350 is aspartic acid (D350), the amino acid residue at position 351 is tryptophan (W351), the amino acid residue at position 363 is threonine (T363), the amino acid residue at position 394 is valine (V394), the amino acid residue at position 451 is valine (V451), and the amino acid residue at position 501 is isoleucine (1501), and the cellobiohydrolase variant comprises mutations at positions T24, T58, D350, W351, T363, V394, V451, and I501. In some embodiments, the variant comprises mutations at positions T24N, T58V, D350E, W351Y, T363D, V394D, V451Y, and I501Q.

In some embodiments, the variant comprises at least about 70% (or at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99%) sequence identity to SEQ ID NO:2 and comprises mutations at positions 58, 350, 351, 363, 394, 451, and 501, wherein the positions are numbered with reference to a wild-type M. thermophila CBH1a protein (e.g., SEQ ID NO:2). In some embodiments, the amino acid residue at position 58 is threonine (T58), the amino acid residue at position 350 is aspartic acid (D350), the amino acid residue at position 351 is tryptophan (W351), the amino acid residue at position 363 is threonine, the amino acid residue at position 394 is valine (V394), the amino acid residue at position 451 is valine (V451), and the amino acid residue at position 501 is isoleucine (1501), and the cellobiohydrolase variant comprises mutations at positions T58, D350, W351, T363, V394, V451, and I501. In some embodiments, the variant comprises mutations at positions T58V, D350E, W351Y, T363D, V394D, V451Y, and 1501Q.

In some embodiments, the variant comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO:2 and comprises at least one mutation at at least one position set forth in Table 2. In some embodiments, the variant comprises at least about 90%, at least about 91%, at least about 92%, at least about 93%, at least about 94%, at least about 95%, at least about 96%, at least about 97%, at least about 98%, or at least about 99% identity to SEQ ID NO:2 and comprises at least one amino acid substitution set selected from the substitution sets listed in Table 2. In some embodiments, the variant comprises at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO:2, comprises one or more amino acid substitution set selected from the substitution sets listed in Table 2, and further comprises at least one, two, three, four, five, six, seven, eight, nine, ten, eleven or twelve additional substitutions, e.g., 1-5, 2-6, 4-8, or 5-12 substitutions (which optionally are conservative substitutions). In some embodiments, the variant has 100% identity to SEQ ID NO:1 except for (1) the substitions present in any individual cellobiohydrolase variant selected from variant numbers 1-473 in Table 2 and (2) one, two, three, four, five, six, seven, eight, nine, ten, eleven or twelve additional substitutions, e.g., 1-5, 2-6, 4-8, or 5-12 substitutions (which optionally are conservative substitutions).

In some embodiments, the variant has at least one improved property relative to wild-type CBH1a (SEQ ID NO:2). In some embodiments, the variant has improved thermoactivity, improved specific activity, and/or improved thermostability relative to wild-type CBH1a (SEQ ID NO:2). In some embodiments, the improved property is improved thermoactivity relative to wild-type CBH1a (SEQ ID NO:2). In some embodiments, the variant exhibits at least a 1.0 fold, at least a 1.1 fold, at least a 1.2 fold, at least a 1.3 fold, at least a 1.4 fold, at least a 1.5 fold or higher increase in thermoactivity relative to wild-type M. thermophila CBH1a (SEQ ID NO:2). In some embodiments, the variant has increased thermoactivity at pH 5.0 and 55° C. in comparison to wild-type M. thermophila CBH1a (SEQ ID NO:2).

In some embodiments, a recombinant cellobiohydrolase variant as described herein is derived from a cellobiohydrolase polypeptide from a fungal strain. In some embodiments, the variant comprises at least about 90%, at least about 91%, at least about 92%, at least about 93%, at least about 94%, at least about 95%, at least about 96%, at least about 97%, at least about 98%, or at least about 99% identity to a cellobiohydrolase type 1 from M. thermophila (SEQ ID NO:2), Thielavia australiensis (SEQ ID NO:4), Humicola grisea (SEQ ID NO:5), Chaetomium thermophilum (SEQ ID NO:6), Sordaria macrospora (SEQ ID NO:7), Chaetomidium pingtungium (SEQ ID NO:8), Botryosphaeria rhodina (SEQ ID NO:9), Trichophaea saccata (SEQ ID NO:10), Aspergillus nidulans (SEQ ID NO:11), Schizophyllum commune (SEQ ID NO:12), or Agaricus bisporus (SEQ ID NO:13).

In another aspect, the present invention provides polynucleotides encoding cellobiohydrolase variants that exhibit improved properties. In some embodiments, the polynucleotide encodes an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprises at least one mutation at position 2, 23, 24, 25, 30, 39, 46, 58, 60, 61, 62, 63, 64, 75, 76, 77, 83, 96, 97, 111, 115, 117, 118, 119, 177, 178, 208, 216, 217, 221, 222, 223, 250, 251, 267, 268, 271, 275, 289, 294, 339, 343, 349, 350, 351, 354, 357, 362, 363, 367, 379, 392, 394, 403, 408, 409, 417, 424, 426, 427, 428, 433, 434, 451, 452, 453, 455, 456, 457, 458, 459, 464, 465, 466, 467, 469, 470, 472, 473, 474, 475, 476, 477, 478, 479, 480, 481, 482, 483, 484, 485, 486, 487, 489, 490, 491, 492, 494, 495, 496, 500, 501, 502, 504, 505, 506, 507, 508, 510, 511, 512, 513, 516, 517, 518, 519, 521, 523, 524, and/or 526, wherein the positions are numbered with reference to a wild-type M. thermophila CBH1a protein (e.g., SEQ ID NO:2). In some embodiments, the polynucleotide encodes an amino acid sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprises at least one mutation at position Y2, L23, T24, A25, S30, G39, Q46, T58, R60, T61, S62, S63, A64, T75, S76, Y77, S83, S96, S97, K111, K115, Q117, Y118, S119, S177, G178, E208, D216, A217, T221, G222, K223, V250, 1251, T267, D268, A271, D275, K289, K294, I339, E343, Q349, D350, W351, R354, A357, V362, T363, D367, A379, H392, V394, W403, A408, G409, A417, V424, A426, E427, V428, P433, N434, V451, S452, G453, P455, D456, G457, G458, S459, P464, P465, V466, S467, S469, T470, V472, P473, S474, S475, S476, T477, T478, S479, S480, G481, S482, S483, G484, P485, T486, G487, T489, G490, V491, A492, H494, Y495, E496, G500, I501, G502, T504, G505, P506, T507, Q508, T510, S511, P512, Y513, T516, K517, L518, N519, W521, S523, Q524, and/or L526, wherein the positions are numbered with reference to a wild-type M. thermophila CBH1a protein (e.g., SEQ ID NO:2). In some embodiments, the polynucleotide encodes an amino acid sequence that comprises at least one mutation at position Y2H, L23E, T24L/N/V, A25D, 530T, G39D, Q46H, T58V/Y, R60T, T61N, S62L, S63A/D, A64P, T75L, S76V, Y77W, S83T/W, S96E/N, S97G, K111Q, K115N, Q117D/E, Y118D, S119D, S177E, G178S, E208G, D216N, A217E, T221E, G222S, K223A/G/L/W, V250I, I251W, T267E/N, D268Y, A271V, D275N, K289D/E/R/W, K294Q, I339V, E343D, Q349T, D350A/E, W351Y, R354L, A357V, V362A/P/T, T363N, D367Y, A379E, H392A/E/P/S, V394A/D, W403Y, A408D/S, G409D, A4175, V424R, A426S, E427N, V428L, P433S, N434E, V451Y, S452E, G453L, P455F/L/Q/W, D456R, G457R, G458A, S459E, P464R, P465A/M/R, V466A/W, S467R, S469C, T470D/E, V472A, P473L/T/W, S474D, S475F/L/R/W, S476P, T477V, T478K/W, S479K, S480M, G481F/P/R/W, S482G, S483G, G484L/P, P485W, T486A/K, G487D/I, T489Y, G490A/W, V491A/P, A492C/V, H494A/E/K/R/W, Y495G, E496L/R/W, G500E/R/V, I501Q/S/W, G502I/L/Q, T504A/G/M, G505E/P, P506A/H/LN, 1507W, Q508W, T510R, S511P/W, P512A/R, Y513G/W, T516A/K/R, K517E/G/P, L518A/E, N519H, W521A, S523A/W, Q524G/P/W, and/or L526E/F/P, wherein the positions are numbered with reference to a wild-type M. thermophila CBH1a protein (e.g., SEQ ID NO:2). In some embodiments, the polynucleotide hybridizes at high stringency to the complement of SEQ ID NO:1 and encodes a cellobiohydrolase variant comprising one or more amino acid substitutions as described herein. In some embodiments, the polynucleotide is operably linked to a heterologous promoter.

In still another aspect, the present invention provides expression vectors comprising a polynucleotide encoding a cellobiohydrolase variant as described herein.

In yet another aspect, the present invention provides host cells comprising a polynucleotide or vector encoding a cellobiohydrolase variant as described herein. In some embodiments, the host cell is transformed with a polynucleotide or vector encoding a cellobiohydrolase variant as described herein. In some embodiments, the host cell expresses a non-naturally occurring cellobiohydrolase having the amino acid sequence of a cellobiohydrolase variant as described herein. In some embodiments, the host cell is a eukaryotic cell. In some embodiments, the host cell is a yeast cell or filamentous fungal cell. In some embodiments, the host cell is selected from Saccharomyces and Myceliopthora. In some embodiments, the host cell further produces at least one other cellulase enzyme in addition to a cellobiohydrolase variant as described herein. In some embodiments, the host cell produces at least one endoglucanase, at least one beta-glucosidase, at least one type 2 cellobiohydrolase, and/or at least one glycoside hydrolase 61 enzyme.

In another aspect, the present invention provides enzyme compositions comprising a recombinant cellobiohydrolase variant as described herein. In some embodiments, the enzyme composition is used in a composition for a saccharification application. In some embodiments, the enzyme composition comprising a cellobiohydrolase variant of the present invention will comprise other recombinant enzymes (e.g., one or more other cellulases).

In still another aspect, the present invention provides methods of producing a cellobiohydrolase variant comprising culturing a host cell transformed with a polynucleotide or vector encoding a cellobiohydrolase variant as described herein under conditions sufficient for the production of the cellobiohydrolase variant by the cell. In some embodiments, the cellobiohydrolase variant polypeptide is secreted by the cell and obtained from the cell culture medium.

In yet another aspect, the present invention provides methods of producing a fermentable sugar, comprising contacting a cellulosic substrate with at least one β-glucosidase (BgI), at least one endoglucanase (EG) such as a type 2 endoglucanase (EG2) and/or a type 1 endoglucanse (EG1, e.g., EG1b), at least one type 2b cellobiohydrolase (CBH2b), at least one CBH1a variant as described herein, and optionally at least one glycoside hydrolase 61 protein (GH61), under conditions in which the fermentable sugar is produced.

In yet another aspect, the present invention provides methods of producing an end-product (e.g., from a cellulosic substrate), comprising (a) contacting the cellulosic substrate with at least one β-glucosidase (BgI), at least one endoglucanase (EG) such as a type 2 endoglucanase (EG2) and/or a type 1 endoglucanse (EG1, e.g., EG1b), at least one type 2b cellobiohydrolase (CBH2b), at least one CBH1a variant as described herein, and optionally at least one glycoside hydrolase 61 protein (GH61), under conditions in which fermentable sugars are produced; and (b) contacting the fermentable sugars with a microorganism in a fermentation to produce the end-product. In some embodiments, prior to step (a), the cellulosic substrate is pretreated to increase its susceptibility to hydrolysis. In some embodiments, the end-product is an alcohol, an amino acid, an organic acid, a diol, or glycerol. In some embodiments, the end-product is an alcohol (e.g., ethanol or butanol). In some embodiments, the microorganism is a yeast. In some embodiments, the process comprises a simultaneous saccharification and fermentation process. In some embodiments, the saccharification and fermentation steps are consecutive. In some embodiments, the enzyme production is simultaneous with saccharification and fermentation.

### BRIEF DESCRIPTION OF THE DRAWINGS

FIG. 1. Amino acid sequence alignment. The amino acid sequence of M. thermophila CBH1a without a signal peptide (SEQ ID NO:2) (“CBH1a”) was aligned with 10 other proteins: Thielavia australiensis unnamed protein (Accession No. CAD79782.1; SEQ ID NO:4), Humicola grisea cellulose (Accession No. BAA09785.1; SEQ ID NO:5), Chaetomium thermophilum cellulose 1,4-beta-cellobiosidase (Accession No. CAM98448.1; SEQ ID NO:6), Sordaria macrospora unnamed protein (Accession No. CBI55936.1; SEQ ID NO:7), Chaetomidium pingtungium unnamed protein (Accession No. CAD79795.1; SEQ ID NO:8), Botryosphaeria rhodina unnamed protein (Accession No. CAD79786.1; SEQ ID NO:9), Trichophaea saccata unnamed protein (Accession No. CAD79787.1.; SEQ ID NO:10), Aspergillus nidulans hypothetical protein AN0494.2 (Accession No. EAA66593.1; SEQ ID NO:11), Schizophyllum commune cellobiohydrolase (Accession No. AAX55505.1; SEQ ID NO:12), or Agaricus bisporus exocellobiohydrolase (Accession No. CAA90422.1; SEQ ID NO:13). Each of the proteins aligned with M. thermophila CBH1a comprises a signal peptide, as shown in FIG. 1A.

### DEFINITIONS

Unless defined otherwise, all technical and scientific terms used herein generally have the same meaning as commonly understood by one of ordinary skill in the art to which this invention belongs. Generally, the nomenclature used herein and the laboratory procedures in analytical chemistry, cell culture, molecular genetics, organic chemistry and nucleic acid chemistry and hybridization described below are those well known and commonly employed in the art. As used herein, “a,” “an,” and “the” include plural references unless the context clearly dictates otherwise.

The terms “biomass,” “biomass substrate,” “cellulosic biomass,” “cellulosic feedstock,” and “cellulosic substrate” refer to materials that contain cellulose. Biomass can be derived from plants, animals, or microorganisms, and may include agricultural, industrial, and forestry residues, industrial and municipal wastes, and terrestrial and aquatic crops grown for energy purposes. Examples of cellulosic substrates include, but are not limited to, wood, wood pulp, paper pulp, corn fiber, corn grain, corn cobs, crop residues such as corn husks, corn stover, grasses, wheat, wheat straw, barley, barley straw, hay, rice, rice straw, switchgrass, waste paper, paper and pulp processing waste, woody or herbaceous plants, fruit or vegetable pulp, distillers grain, rice hulls, cotton, hemp, flax, sisal, sugar cane bagasse, sugar beets, beet pulp, sorghum, soy, components obtained from milling of grains, trees, branches, roots, leaves, wood chips, sawdust, shrubs and bushes, vegetables, fruits, and flowers and mixtures thereof. In some embodiments, the biomass or cellulosic substrate comprises, but is not limited to, cultivated crops (e.g., grasses, including C4 grasses, such as switchgrass, cord grass, rye grass, miscanthus, reed canary grass, or any combination thereof), sugar processing residues, for example, but not limited to, bagasse (e.g., sugar cane bagasse, beet pulp [e.g., sugar beet], or a combination thereof), agricultural residues (e.g., soybean stover, corn stover, corn fiber, rice straw, sugar cane straw, rice, rice hulls, barley straw, corn cobs, wheat straw, canola straw, oat straw, oat hulls, hemp, flax, sisal, cotton, or any combination thereof), fruit pulp, vegetable pulp, distillers' grains, and/or forestry biomass (e.g., wood, wood pulp, paper pulp, recycled wood pulp fiber, sawdust, hardwood, such as aspen wood, softwood, or a cominbation thereof). Furthermore, in some embodiments, the biomass or cellulosic substrate comprises cellulosic waste material and/or forestry waste materials, including but not limited to, paper and pulp processing waste, municipal paper waste, newsprint, cardboard, and the like. In some embodiments, biomass comprises one species of fiber, while in some alternative embodiments, the biomass or cellulosic substrate comprises a mixture of fibers that originate from different biomasses. In some embodiments, the biomass may also comprise transgenic plants that express ligninase and/or cellulase enzymes, see, e.g., US 2008/0104724. In some embodiments, the biomass substrate is “pretreated” using methods known in the art, such as chemical pretreatment (e.g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms) and combinations thereof, to increase the susceptibility of cellulose to hydrolysis.

“Saccharification” refers to the process in which substrates (e.g., cellulosic biomass) are broken down via the action of cellulases to produce fermentable sugars (e.g. monosaccharides such as but not limited to glucose).

“Fermentable sugars” refers to simple sugars (monosaccharides, disaccharides and short oligosaccharides) such as but not limited to glucose, xylose, galactose, arabinose, mannose and sucrose. Fermentable sugar is any sugar that a microorganism can utilize or ferment.

The term “fermentation” is used broadly to refer to the cultivation of a microorganism or a culture of microorganisms that use simple sugars, such as fermentable sugars, as an energy source to obtain a desired product.

As used herein, the term “cellulase” refers to a category of enzymes capable of hydrolyzing cellulose (β-1,4-glucan or β-D-glucosidic linkages) to shorter cellulose chains, oligosaccharides, cellobiose, and/or glucose.

As used herein, the term “cellobiohydrolase” or “CBH” refers to a category of cellulases (EC 3.2.1.91) that hydrolyze glycosidic bonds in cellulose. In some embodiments, the cellobiohydrolase is a “type 1 cellobiohydrolase,” a cellobiohydrolase belonging to the glycoside hydrolase family 7 (GH7) family of cellulases and which is also commonly called “the Cel7 family.” Cellobiohydrolases of the GH7 family are described, for example, in the Carbohydrate Active Enzymes (CAZY) database, accessible at www.cazy.org/GH7.html. Recombinant CBH variants have been described in the art.

As used herein, the term “endoglucanase” or “EG” refers to a category of cellulases (EC 3.2.1.4) that catalyze the hydrolysis of internal β-1,4 glucosidic bonds of cellulose. Recombinant EG variants have been described in the art.

As used herein, the term “β-glucosidase,” “cellobiase,” or “BGL” refers to a category of cellulases (EC 3.2.1.21) that catalyze the hydrolysis of cellobiose to glucose. Recombinant BGL variants have been described in the art. Exemplary BGLs include, but are not limited to, BGLs described in U.S. Pat. Nos. 8,143,050 and 8,323,947.

As used herein, the term “glycoside hydrolase 61” or “GH61” refers to a category of cellulases that enhance cellulose hydrolysis when used in conjunction with one or more additional cellulases. The GH61 family of cellulases is described, for example, in the Carbohydrate Active Enzymes (CAZY) database, accessible at www.cazy.org/GH61.html, and in Harris et al., 2010, Biochemistry 49(15):3305-16. Recombinant GH61 variants have been described in the art. Exemplary GH61 enzymes include, but are not limited to, those GH61 enzymes described in U.S. Pat. No. 8,298,795.

As used herein, the term “C1” refers to Myceliophthora thermophila, including a fungal strain described by Garg, A., 1966, “An addition to the genus Chrysosporium corda” Mycopathologia 30: 3-4. “Chrysosporium lucknowense” includes the strains described in U.S. Pat. Nos. 5,811,381, 6,015,707, 6,573,086, 8,236,551, and 8,309,328; US Pat. Pub. Nos. 2007/0238155, US 2008/0194005, US 2009/0099079; International Pat. Pub. Nos., WO 2008/073914 and WO 98/15633, all incorporated herein by reference, and include, without limitation, Chrysosporium lucknowense Garg 27K, VKM-F 3500 D (Accession No. VKM F-3500-D), C1 strain UVβ-6 (Accession No. VKM F-3632 D), C1 strain NG7C-19 (Accession No. VKM F-3633 D), and C1 strain UV18-25 (VKM F-3631 D), all of which have been deposited at the All-Russian Collection of Microorganisms of Russian Academy of Sciences (VKM), Bakhurhina St. 8, Moscow, Russia, 113184, and any derivatives thereof. Although initially described as Chlysosporium lucknowense, C1 may currently be considered a strain of Myceliophthora thermophila. Other C1 strains include cells deposited under accession numbers ATCC 44006 and PTA-122255, CBS (Centraalbureau voor Schimmelcultures) 122188, CBS 251.72, CBS 143.77, CBS 272.77, CBS122190, CBS122189, and VKM F-3500D. Exemplary C1 derivatives include modified organisms in which one or more endogenous genes or sequences have been deleted or modified and/or one or more heterologous genes or sequences have been introduced. Derivatives include UV18#100f Δpyr5 Δalp1 Δalpl, UV18#100f Δpyr5 Δalp1, UV18#100.f Δalp1 Δpep4 Δalp2, UV18#100.f Δpyr5 Δalp1 Δpep4 Δalp2 and UV18#100.f Δpyr4 Δpyr5 Δalp1 Δpep4 Δalp2, as described in WO2008073914 and WO2010107303, each of which is incorporated herein by reference.

As used herein, the term “wild-type M. thermophila cellobiohydrolase type 1a” or “wild-type M. thermophila CBH1a” refers to the polypeptide sequence provided herein as SEQ ID NO:2. SEQ ID NO:2 is the mature peptide sequence (i.e., lacking a signal peptide) of CBH1a that is expressed by the naturally occurring fungal strain M. thermophila.

As used herein, the term “variant” refers to a cellobiohydrolase polypeptide or polynucleotide encoding a cellobiohydrolase polypeptide comprising one or more modifications relative to wild-type M. thermophila CBH1a or the wild-type polynucleotide encoding M. thermophila CBH1a (such as substitutions, insertions, deletions, and/or truncations of one or more amino acid residues or of one or more specific nucleotides or codons in the polypeptide or polynucleotide, respectively). In some embodiments, the variant is a “M. thermophila variant” derived from a M. thermophila CBH1a polypeptide and comprising one or more modifications relative to wild-type M. thermophila CBH1a or the wild-type polynucleotide encoding wild-type M. thermophila CBH1a. In some embodiments, reference to a “modification” or a “mutation” at an amino acid residue refers to a substitution of the amino acid residue for another amino acid residue.

As used herein, the term “cellobiohydrolase polypeptide” refers to a polypeptide having cellobiohydrolase activity.

As used herein, the term “cellobiohydrolase polynucleotide” refers to a polynucleotide encoding a polypeptide having cellobiohydrolase activity.

As used herein, the term “cellobiohydrolase activity” refers to the enzymatic activity of a cellobiohydrolase, e.g., hydrolyzing a cellulose-containing substrate.

The terms “improved” or “improved properties,” as used in the context of describing the properties of a cellobiohydrolase variant, refers to a cellobiohydrolase variant polypeptide that exhibits an improvement in a property or properties as compared to the wild-type M. thermophila CBH1a (SEQ ID NO:2) or a specified reference polypeptide. Improved properties may include increased protein expression, increased thermoactivity, increased thermostability, increased pH activity, increased stability (e.g., increased pH stability), increased product specificity, increased specific activity, increased substrate specificity, increased resistance to substrate or end-product inhibition, increased chemical stability, reduced inhibition by glucose, increased resistance to inhibitors (e.g., acetic acid, lectins, tannic acids, and phenolic compounds), and altered pH/temperature profile.

As used herein, the term “improved thermoactivity” or “increased thermoactivity,” refers to a variant enzyme displaying an increase, relative to a reference enzyme (e.g., a wild-type cellobiohydrolase), in the amount of cellobiohydrolase enzymatic activity (e.g., substrate hydrolysis) in a specified time under specified reaction conditions. Exemplary methods for measuring cellobiohydrolase activity are provided in the Examples and include, but are not limited to, measuring cellobiose production from crystalline cellulose or from pretreated biomass as measured by colorimetric assay or HPLC. To compare cellobiohydrolase activity of two recombinantly expressed proteins, the specific activity (activity per mole enzyme or activity per gram enzyme) can be compared. Alternatively, cells expressing and secreting the recombinant proteins can be cultured under the same conditions and the cellobiohydrolase activity per volume culture medium can be compared.

As used herein, the term “improved thermostability” or “increased thermostability” refers to a variant enzyme displaying an increase in “residual activity” relative to a reference enzyme (e.g., a wild-type cellobiohydrolase). Residual activity is determined by (1) exposing the variant enzyme or wild-type enzyme to stress conditions of elevated temperature, optionally at lowered pH, for a period of time and then determining cellobiohydrolase activity; (2) exposing the variant enzyme or wild-type enzyme to unstressed conditions for the same period of time and then determining cellobiohydrolase activity; and (3) calculating residual activity as the ratio of activity obtained under stress conditions (1) over the activity obtained under unstressed conditions (2). For example, the cellobiohydrolase activity of the enzyme exposed to stress conditions (“a”) is compared to that of a control in which the enzyme is not exposed to the stress conditions (“b”), and residual activity is equal to the ratio a/b. A variant with increased thermostability will have greater residual activity than the wild-type enzyme. In one embodiment the enzymes are exposed to stress conditions of 66° C. at pH 4.4 for 2 hr, but other cultivation conditions, such as conditions described herein, can be used.

As used herein, the term “reference enzyme” refers to an enzyme to which a variant enzyme of the present invention is compared in order to determine the presence of an improved property in the variant enzyme being evaluated, including but not limited to improved thermoactivity, improved specific activity, or improved thermostability. In some embodiments, a reference enzyme is a wild-type enzyme (e.g., wild-type M. thermophila CBH1a). In some embodiments, a reference enzyme is another variant enzyme (e.g., another variant enzyme of the present invention).

As used herein, “polynucleotide” refers to a polymer of deoxyribonucleotides or ribonucleotides in either single- or double-stranded form, and complements thereof.

Nucleic acids “hybridize” when they associate, typically in solution. Nucleic acids hybridize due to a variety of well-characterized physico-chemical forces, such as hydrogen bonding, solvent exclusion, base stacking and the like. As used herein, the term “stringent hybridization wash conditions” in the context of nucleic acid hybridization experiments, such as Southern and Northern hybridizations, are sequence dependent, and are different under different environmental parameters. Guidance about hybridization of nucleic acids is found, for example, in Tijssen, 1993, “Laboratory Techniques in Biochemistry and Molecular Biology-Hybridization with Nucleic Acid Probes,” Part I, Chapter 2 (Elsevier, New York), which is incorporated herein by reference. For polynucleotides of at least 100 nucleotides in length, low to very high stringency conditions are defined as follows: prehybridization and hybridization at 42° C. in 5×SSPE, 0.3% SDS, 200 μg/ml sheared and denatured salmon sperm DNA, and either 25% formamide for low stringencies, 35% formamide for medium and medium-high stringencies, or 50% formamide for high and very high stringencies, following standard Southern blotting procedures. For polynucleotides of at least 100 nucleotides in length, the carrier material is finally washed three times each for 15 minutes using 2×SSC, 0.2% SDS 50° C. (low stringency), at 55° C. (medium stringency), at 60° C. (medium-high stringency), at 65° C. (high stringency), or at 70° C. (very high stringency).

The terms “peptide,” “polypeptide,” and “protein” are used interchangeably herein to refer to a polymer of amino acid residues.

The term “pre-protein” refers to a protein including an amino-terminal signal peptide (or leader sequence) region attached. The signal peptide is cleaved from the pre-protein by a signal peptidase prior to secretion to result in the “mature” or “secreted” protein.

The term “amino acid” refers to naturally occurring and synthetic amino acids, as well as amino acid analogs. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

A “conservative substitution” as used with respect to amino acids, refers to the substitution of an amino acid with a chemically similar amino acid. Amino acid substitutions which often preserve the structural and/or functional properties of the polypeptide in which the substitution is made are known in the art and are described, for example, by H. Neurath and R. L. Hill, 1979, in “The Proteins,” Academic Press, New York. The most commonly occurring exchanges are isoleucine/valine, tyrosine/phenylalanine, aspartic acid/glutamic acid, lysine/arginine, methionine/leucine, aspartic acid/asparagine, glutamic acid/glutamine, leucine/isoleucine, methionine/isoleucine, threonine/serine, tryptophan/phenylalanine, tyrosine/histidine, tyrosine/tryptophan, glutamine/arginine, histidine/asparagine, histidine/glutamine, lysine/asparagine, lysine/glutamine, lysine/glutamic acid, phenylalanine/leucine, phenylalanine/methionine, serine/alanine, serine/asparagine, valine/leucine, and valine/methionine. In some embodiments, there may be at least 1, at least 2, at least 3, at least 4, at least 5, at least 6, at least 7, at least 8, at least 9, at least 10, at least 15, at least 20, at least 25, at least 30, at least 35, or at least 40 conservative substitutions. In some embodiments, the number of conservative substitutions does not exceed 1% of the number of amino acid residues in the protein (e.g., does not exceed 5 substitutions to the CBH1a of SEQ ID NO:2), does not exceed 2% of the number of amino acid residues in the protein (e.g., does not exceed 10 substitutions to the CBH1a of SEQ ID NO:2), does not exceed 3% of the number of amino acid residues in the protein (e.g., does not exceed 15 substitutions to the CBH1a of SEQ ID NO:2), does not exceed 4% of the number of amino acid residues in the protein (e.g., does not exceed 20 substitutions to the CBH1a of SEQ ID NO:2), or does not exceed 5% of the number of amino acid residues in the protein (e.g., does not exceed 25 substitutions to the CBH1a of SEQ ID NO:2).

The following nomenclature may be used to describe substitutions in a reference sequence relative to a reference sequence or a variant polypeptide or nucleic acid sequence: “R-#-V,” where # refers to the position in the reference sequence, R refers to the amino acid (or base) at that position in the reference sequence, and V refers to the amino acid (or base) at that position in the variant sequence. In some embodiments, an amino acid (or base) may be called “X,” by which is meant any amino acid (or base). As a non-limiting example, for a variant polypeptide described with reference to a wild-type cellobiohydrolase type 1a protein of SEQ ID NO:2, “T24L” indicates that in the variant polypeptide, the threonine at position 24 of the reference sequence is replaced by leucine, with amino acid position being determined by optimal alignment of the variant sequence with SEQ ID NO:2. Similarly, “T24L/N/V” describes three variants: a variant in which the threonine at position 24 of the reference sequence is replaced by leucine, a variant in which the threonine at position 24 of the reference sequence is replaced by asparagine, and a variant in which the threonine at position 24 of the reference sequence is replaced by valine.

The term “amino acid substitution set” or “substitution set” refers to a group of amino acid substitutions. A substitution set can have 1, 2, 3, 4, 5, 6, 7, 8, 9, 10, 11, 12, 13, 14, 15, or more amino acid substitutions. In some embodiments, a substitution set refers to the set of amino acid substitutions that is present in any of the variant cellobiohydrolases listed in Table 2. For example, the substitution set for Variant 207 (Table 2) consists of the amino acid substitutions T24N, A379E, and V394D.

The term “isolated” refers to a nucleic acid, polynucleotide, polypeptide, protein, or other component that is partially or completely separated from components with which it is normally associated (other proteins, nucleic acids, cells, etc.). In some embodiments, an isolated polypeptide or protein is a recombinant polypeptide or protein.

A nucleic acid (such as a polynucleotide), a polypeptide, or a cell is “recombinant” when it is artificial or engineered, or derived from or contains an artificial or engineered protein or nucleic acid. For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Identity” or “percent identity,” in the context of two or more polypeptide sequences, refers to two or more sequences or subsequences that are the same or have a specified percentage of amino acid residues that are the same (e.g., share at least about 70%, at least about 75%, at least about 80%, at least about 85%, at least about 88% identity, at least about 89%, at least about 90%, at least about 91%, at least about 92%, at least about 93%, at least about 94%, at least about 95%, at least about 96%, at least about 97%, at least about 98%, or at least about 99% identity) over a specified region to a reference sequence, when compared and aligned for maximum correspondence over a comparison window, or designated region as measured using a sequence comparison algorithms or by manual alignment and visual inspection.

Optimal alignment of sequences for comparison and determination of sequence identity can be determined by a sequence comparison algorithm or by visual inspection (see, generally, Current Protocols in Molecular Biology, F. M. Ausubel et al., eds., Current Protocols, a joint venture between Greene Publishing Associates, Inc. and John Wiley & Sons, Inc., (supplemented through 2009) (“Ausubel”)). When optimally aligning sequences and determining sequence identity by visual inspection, percent sequence identity is calculated as the number of residues of the test sequence that are identical to the reference sequence divided by the number of non-gap positions and multiplied by 100. When using a sequence comparison algorithm, test and reference sequences are entered into a computer, subsequence coordinates and sequence algorithm program parameters are designated. The sequence comparison algorithm then calculates the percent sequence identities for the test sequences relative to the reference sequence, based on the program parameters.

An algorithm that may be used to determine whether a variant cellobiohydrolase has sequence identity to SEQ ID NO:2 is the BLAST algorithm, which is described in Altschul et al., 1990, J. Mol. Biol. 215:403-410, which is incorporated herein by reference. Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. For amino acid sequences, the BLASTP program uses as defaults a word size (W) of 3, an expectation (E) of 10, and the BLOSUM62 scoring matrix (see Henikoff & Henikoff, 1989, Proc. Natl. Acad. Sci. USA 89:10915). Other programs that may be used include the Needleman-Wunsch procedure, J. Mol. Biol. 48: 443-453 (1970), using blosum62, a Gap start penalty of 7 and gap extend penalty of 1; and gapped BLAST 2.0 (see Altschul, et al. 1997, Nucleic Acids Res., 25:3389-3402) both available to the public at the National Center for Biotechnology Information Website.

Multiple sequences can be aligned with each other by visual inspection or using a sequence comparison algorithm, such as PSI-BLAST (Altschul, et al., 1997, supra) or “T-Coffee” (Notredame et al., 2000, J. Mol. Bio. 302:205-17). T-Coffee alignments may be carried out using default parameters (T-Coffee Technical Documentation, Version 8.01, July 2009, WorldWideWeb .tcoffee.org), or Protein Align. In Protein Align, alignments are computed by optimizing a function based on residue similarity scores (obtained from applying an amino acid substitution matrix to pairs of aligned residues) and gap penalties. Penalties are imposed for introducing an extending gaps in one sequence with respect to another. The final optimized function value is referred to as the alignment score. When aligning multiple sequences, Protein Align optimizes the “sum of pairs” score, i.e., the sum of all the separate pairwise alignment scores.

The phrase “substantial sequence identity” or “substantial identity,” in the context of two nucleic acid or polypeptide sequences, refers to a sequence that has at least 70% identity to a reference sequence. Percent identity can be any integer from 70% to 100%. Two nucleic acid or polypeptide sequences that have 100% sequence identity are said to be “identical.” A nucleic acid or polypeptide sequence are said to have “substantial sequence identity” to a reference sequence when the sequences have at least about 70%, at least about 75%, at least 80%, at least 85%, at least 90%, at least 91%, at least 92%, at least 93%, at least 94%, at least 95%, at least 96%, at least 97%, at least 98%, or at least 99% or greater sequence identity as determined using the methods described herein, such as BLAST using standard parameters as described above. For an alignment that extends along the entire length of SEQ ID NO:2, there may be at least 357, at least 382, at least 407, at least 433, at least 459, at least 464, at least 469, at least 474, at least 479, at least 484, at least 489, at least 494, at least 499, or at least 504 amino acids identical between a variant sequence and SEQ ID NO:2.

A “vector” is a DNA construct for introducing a DNA sequence into a cell. A vector may be an expression vector that is operably linked to a suitable control sequence capable of effecting the expression in a suitable host of the polypeptide encoded in the DNA sequence. An “expression vector” has a promoter sequence operably linked to the DNA sequence (e.g., transgene) to drive expression in a host cell, and in some embodiments a transcription terminator sequence.

The term “expression” includes any step involved in the production of the polypeptide including, but not limited to, transcription, post-transcriptional modification, translation, post-translational modification, and secretion.

The term “operably linked” refers to a configuration in which a control sequence is appropriately placed at a position relative to the coding sequence of the DNA sequence such that the control sequence influences the expression of a polypeptide.

An amino acid or nucleotide sequence (e.g., a promoter sequence, signal peptide, terminator sequence, etc.) is “heterologous” to another sequence with which it is operably linked if the two sequences are not associated in nature.

The terms “transform” or “transformation,” as used in reference to a cell, means a cell has a non-native nucleic acid sequence integrated into its genome or as an episome (e.g., plasmid) that is maintained through multiple generations.

The term “culturing” refers to growing a population of microbial cells under suitable conditions in a liquid or solid medium.

The term “introduced,” as used in the context of inserting a nucleic acid sequence into a cell, means conjugated, transfected, transduced or transformed (collectively “transformed”) or otherwise incorporated into the genome of, or maintained as an episome in, the cell.

### DETAILED DESCRIPTION OF THE INVENTION

### I. Introduction

Fungi, bacteria, and other organisms produce a variety of cellulases and other enzymes that act in concert to catalyze decrystallization and hydrolysis of cellulose to yield fermentable sugars. One such fungus is M. thermophila, which was described hereinabove. One M. thermophila cellulase of interest is the cellobiohydrolase referred to as “M. thermophila cellobiohydrolase type 1a” or “CBH1a.”

The cellobiohydrolase variants described herein are particularly useful for the production of fermentable sugars from cellulosic biomass. In one aspect, the present invention relates to cellobiohydrolase variants that have improved properties, relative to wild-type M. thermophila CBH1a (SEQ ID NO:2), under process conditions used for saccharification of biomass. Improved properties, described herein, include improved thermoactivity, improved specific activity, and/or improved thermostability. In another aspect, the invention provides a host cell comprising a recombinant nucleic acid sequence encoding a variant cellobiohydrolase. In another aspect, the present invention provides a method for expressing a variant cellobiohydrolase by maintaining the cell under conditions in which the cellobiohydrolase protein is expressed and, preferably, secreted. In another aspect, the present invention provides methods of generating fermentable sugars from cellulosic biomass, by contacting the biomass with a cellulase composition comprising a CBH1a variant as described herein under conditions suitable for the production of fermentable sugars. As described in greater detail below, recombinant host cells expressing cellobiohydrolase variants of the invention may be combined with a cellulosic biomass or other cellobiohydrolase substrate under conditions in which the cellobiohydrolase is expressed and/or secreted by the cells as part of a saccharification process. Alternatively, a substantially or partially purified recombinant cellobiohydrolase protein may be contacted with cellulosic biomass or other cellobiohydrolase substrate.

Various aspects of the invention are described in the following sections.

### II. Cellobiohydrolase Variants

In one aspect, the present invention provides M. thermophila CBH1a variants having improved properties over a wild-type cellobiohydrolase. In some embodiments, the CBH1a variants of the present invention exhibit improved thermoactivity, improved specific activity, and/or improved thermostability in comparison to a wild-type type 1 cellobiohydrolase (e.g., a M. thermophila CBH1a having the amino acid sequence of SEQ ID NO:2) under conditions relevant to commercial cellulose hydrolysis processes.

In some embodiments, the present invention provides a recombinant M. thermophila CBH1a variant comprising at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprising an amino acid substitution at one or more positions selected from Y2, L23, T24, A25, S30, G39, Q46, T58, R60, T61, S62, S63, A64, T75, S76, Y77, S83, S96, S97, K111, K115, Q117, Y118, S119, S177, G178, E208, D216, A217, T221, G222, K223, V250, I251, T267, D268, A271, D275, K289, K294, I339, E343, Q349, D350, W351, R354, A357, V362, T363, D367, A379, H392, V394, W403, A408, G409, A417, V424, A426, E427, V428, P433, N434, V451, S452, G453, P455, D456, G457, G458, S459, P464, P465, V466, S467, S469, T470, V472, P473, S474, S475, S476, T477, T478, S479, S480, G481, S482, S483, G484, P485, T486, G487, T489, G490, V491, A492, H494, Y495, E496, G500, I501, G502, T504, G505, P506, T507, Q508, T510, S511, P512, Y513, T516, K517, L518, N519, W521, S523, Q524, and L526, wherein the position is numbered with reference to SEQ ID NO:2, and wherein the variant has increased thermoactivity in comparison to wild-type M. thermophila CBH1a (SEQ ID NO:2). In some embodiments, a CBH1a variant of the present invention has an amino acid sequence that is encoded by a nucleic acid that hybridizes under stringent conditions to the complement of SEQ ID NO:1 (e.g., over substantially the entire length of a nucleic acid exactly complementary to SEQ ID NO:1) and comprises an amino acid substitution at one or more positions selected from Y2, L23, T24, A25, S30, G39, Q46, T58, R60, T61, S62, S63, A64, T75, S76, Y77, S83, S96, S97, K111, K115, Q117, Y118, S119, S177, G178, E208, D216, A217, T221, G222, K223, V250, I251, T267, D268, A271, D275, K289, K294, I339, E343, Q349, D350, W351, R354, A357, V362, T363, D367, A379, H392, V394, W403, A408, G409, A417, V424, A426, E427, V428, P433, N434, V451, S452, G453, P455, D456, G457, G458, S459, P464, P465, V466, S467, S469, T470, V472, P473, S474, S475, S476, T477, T478, S479, S480, G481, S482, S483, G484, P485, T486, G487, T489, G490, V491, A492, H494, Y495, E496, G500, I501, G502, T504, G505, P506, T507, Q508, T510, S511, P512, Y513, T516, K517, L518, N519, W521, S523, Q524, and L526, wherein the position is numbered with reference to SEQ ID NO:2.

In some embodiments, the present invention provides a recombinant M. thermophila CBH1a variant comprising at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprising one or more amino acid substitutions selected from Y2H, L23E, T24L/N/V, A25D, S30T, G39D, Q46H, T58Y, R60T, T61N, S62L, S63A/D, A64P, T75L, S76V, Y77W, S83T/W, S96E/N, S97G, K111Q, K115N, Q117D/E, Y118D, S119D, S177E, G178S, E208G, D216N, A217E, T221E, G222S, K223A/G/L/W, V250I, I251W, T267E, D268Y, A271V, D275N, K289D/E/R/W, K294Q, I339V, E343D, Q349T, D350A, W351Y, R354L, A357V, V362A/P/T, T363N, D367Y, A379E, H392E/P/S, V394A/D, W403Y, A408D/S, G409D, A417S, V424R, A426S, E427N, V428L, P433S, N434E, V451Y, S452E, G453L, P455F/L/Q/W, D456R, G457R, G458A, S459E, P464R, P465A/M/R, V466A/W, S467R, S469C, T470D/E, V472A, P473L/T/W, S474D, S475F/L/R/W, S476P, T477V, T478K/W, S479K, S480M, G481F/P/R/W, S482G, S483G, G484L/P, P485W, T486A, G487D/I, T489Y, G490A/W, V491A/P, A492CN, H494A/E/K/R/W, Y495G, E496UR/W, G500E/RN, 1501Q/S/W, G502I/L/Q, T504A/G/M, G505E/P, P506A/H/L/V, T507W, Q508W, T510R, S511P/W, P512A, Y513G/W, T516A/K/R, K517E/G/P, L518A/E, N519H, W521A, S523A1W, Q524G/P/W, and L526E/F/P, wherein the position is numbered with reference to SEQ ID NO:2. In some embodiments, a CBH1a variant of the present invention has an amino acid sequence that is encoded by a nucleic acid that hybridizes under stringent conditions to the complement of SEQ ID NO:1 (e.g., over substantially the entire length of a nucleic acid exactly complementary to SEQ ID NO:1) and comprises one or more amino acid substitutions selected from Y2H, L23E, T24L/N/V, A25D, S30T, G39D, Q46H, T58Y, R60T, T61N, S62L, S63A/D, A64P, T75L, S76V, Y77W, S83T/W, S96E/N, S97G, K111Q, K115N, Q117D/E, Y118D, S119D, S177E, G178S, E208G, D216N, A217E, T221E, G222S, K223A/G/LNV, V2501,1251W, T267E, D268Y, A271V, D275N, K289D/E/R/W, K294Q, 1339V, E343D, Q349T, D350A, W351Y, R354L, A357V, V362A/P/T, T363N, D367Y, A379E, H392E/P/S, V394A/D, W403Y, A408D/S, G409D, A417S, V424R, A426S, E427N, V428L, P433S, N434E, V451Y, S452E, G453L, P455F/L/Q/W, D456R, G457R, G458A, S459E, P464R, P465A/M/R, V466A/W, S467R, S469C, T470D/E, V472A, P473UT/W, S474D, S475F/L/R/W, S476P, T477V, T478K/W, S479K, S480M, G481F/P/R/W, S482G, S483G, G484L/P, P485W, T486A, G487D/I, T489Y, G490A/W, V491A/P, A492C/V, H494A/E/K/R/W, Y495G, E496L/R/W, G500E/R/V, 1501Q/S/W, G502I/L/Q, T504A/G/M, G505E/P, P506A/H/L/V, T507W, Q508W, T510R, S511PNV, P512A, Y513G/W, T516A/K/R, K517E/G/P, L518A/E, N519H, W521A, S523A/W, Q524G/P/W, and L526E/F/P, wherein the position is numbered with reference to SEQ ID NO:2.

In some embodiments, a CBH1a variant of the present invention exhibits at least about a 1.1 fold, at least about a 1.2 fold, at least about a 1.3 fold, at least about a 1.4 fold, or at least about a 1.5 fold increase or more in thermoactivity relative to wild-type M. thermophila CBH1a (SEQ ID NO:2). Exemplary variants are identified in Table 2, wherein fold improvement in thermoactivity is measured as described in the Examples (e.g., expressed in S. cerevisiae).

In some embodiments, a CBH1a variant comprises at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more amino acid residues which have been substituted (e.g., with substitutions described herein) as compared to the amino acid sequence of the wild-type cellobiohydrolase protein from which the cellobiohydrolase variant is derived.

In some embodiments, a CBH1a variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprises one or more amino acid substitution sets selected from the substitution sets identified in Table 2. In some embodiments, a CBH1a variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2, comprises one or more amino acid substitution sets selected from the substitution sets identified in Table 2, and further comprises at least 1, 2, 3, 4, 5, 6, 7, 8, 9, 10, or more additional amino acid substitutions. In some embodiments, a CBH1a variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprises an amino acid substitution set selected from the substitution sets of any of Variants 24, 207, 208, 251, 472, or 473 as identified in Table 2. In some embodiments, a CBH1a variant comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprises an amino acid substitution set selected from the substitution sets of any of Variants 11, 12, 14, 19, 24, 25, 27, 167, 180, or 182 as identified in Table 2.

In some embodiments, a CBH1a variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprises an amino acid substitution set selected from the substitution sets showing at least 1.0 to 1.2 fold, at least 1.3 to 1.5 fold, or higher improvement in thermoactivity over wild-type M. thermophila CBH1a (SEQ ID NO:2), as identified in Table 2.

In some embodiments, a CBH1a variant of the present invention comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprises an amino acid substitution set selected from the substitution sets showing at least 1.0 to 1.2 fold, at least 1.3 to 1.5 fold, or higher improvement in thermoactivity over cellobiohydrolase Variant 207 (SEQ ID NO:19), Variant 208 (SEQ ID NO:20), or Variant No. 472 (SEQ ID NO:22) as identified in Table 2.

Certain cellobiohydrolase variants comprise an amino acid substitution at one or more positions selected from T24 and A379. In some embodiments, a cellobiohydrolase variant comprises one or more amino acid substitutions selected from T24V and A379E.

Certain cellobiohydrolase variants comprise an amino acid substitution at one or more positions selected from T24, A379, and V394. In some embodiments, a cellobiohydrolase variant comprises one or more amino acid substitutions selected from T24N, A379E, and V394D. In some embodiments, a cellobiohydrolase variant comprises the amino acid substitution set of T24N, A379E, and V394D. In some embodiments, the cellobiohydrolase variant has the amino acid sequence of SEQ ID NO:19.

Certain cellobiohydrolase variants comprise an amino acid substitution at one or more positions selected from T24, D350, W351, and V394. In some embodiments, a cellobiohydrolase variant comprises one or more amino acid substitutions selected from T24N, T24V, D350E, W351Y, and V394D. In some embodiments, a cellobiohydrolase variant comprises the amino acid substitution set of T24V, D350E, W351Y, and V394D. In some embodiments, a cellobiohydrolase variant comprises the amino acid substitution set of T24N, D350E, W351Y, and V394D. In some embodiments, the cellobiohydrolase variant has the amino acid sequence of SEQ ID NO:20.

Certain cellobiohydrolase variants comprise an amino acid substitution at position Y77. In some embodiments, a cellobiohydrolase variant comprises the amino acid substitution Y77W. In some embodiments, the cellobiohydrolase variant has the amino acid sequence of SEQ ID NO:18.

Certain cellobiohydrolase variants comprise an amino acid substitution at one or more positions selected from T221 and E427. In some embodiments, a cellobiohydrolase variant comprises one or more amino acid substitutions selected from T221E and E427N. In some embodiments, the cellobiohydrolase variant has the amino acid sequence of SEQ ID NO:21.

Certain cellobiohydrolase variants comprise an amino acid substitution at one or more positions selected from T24, T58, D350, W351, T363, V394, V451, and 1501. In some embodiments, a cellobiohydrolase variant comprises one or more amino acid substitutions selected from T24N, T58V, D350E, W351Y, T363D, V394D, V451Y, and I501Q. In some embodiments, a cellobiohydrolase variant comprises the amino acid substitution set of T24N, T58V, D350E, W351Y, T363D, V394D, V451Y, and I501Q. In some embodiments, the cellobiohydrolase variant has the amino acid sequence of SEQ ID NO:22.

Certain cellobiohydrolase variants comprise an amino acid substitution at one or more positions selected from T58, D350, W351, T363, V394, V451, and I501. In some embodiments, a cellobiohydrolase variant comprises one or more amino acid substitutions selected from T58V, D350E, W351Y, T363D, V394D, V451Y, and I501Q. In some embodiments, a cellobiohydrolase variant comprises the amino acid substitution set of T58V, D350E, W351Y, T363D, V394D, V451Y, and I501Q. In some embodiments, the cellobiohydrolase variant has the amino acid sequence of SEQ ID NO:23.

One of skill in the art will recognize that the disclosures in the paragraphs herein of the properties and/or characteristics of cellobiohydrolase variants can be combined. As a non-limiting example, a cellobiohydrolase variant can have one or more substitution sets as described herein and can further have the property of improved thermoactivity relate to a reference protein as described herein.

It will be appreciated that secreted cellobiohydrolase variants of the present invention may encompass additional amino acid substitutions beyond those listed above (such as additional conservative substitutions) may be less-than-full length compared to a wild-type secreted M. thermophila cellobiohydrolase protein and may contain additional residues at the amino and/or carboxy termini. Thus, cellobiohydrolase variants of the present invention may comprise insertions or deletions (e.g., truncation at the amino- and/or carboxy-termini) relative to SEQ ID NO:2. For illustration and not limitation, in some embodiments the variant may be longer or shorter by up to about 25%, about 20%, about 15%, about 10%, about 5%, about 4%, about 3%, about 2%, or about 1% of the wild-type length.

In some embodiments, a cellobiohydrolase variant of the present invention is truncated at the C-terminus and/or has a disruption of the cellulose binding domain (CBD). Without being bound to a particular theory, truncation of the C-terminus may disrupt the folding of the CBD and/or affect the ability of the CBD to bind substrate. Accordingly, in one aspect the present invention provides a CBH1a variant wherein the CBD, or a substantial portion of the CBD has been modified to disrupt folding and/or template binding. Such a modified CBD or deleted CBD is likely to beneficial for cellobiohydrolase properties, e.g., thermostability and/or tolerance for low pH.

In some embodiments, one or more modifications to the CBD is combined with one or more substitutions described herein (e.g., one or more amino acid substitutions or one or more substitution sets listed in Table 2).

The M. thermophila CBH1a CBD comprises residues 474-509 of SEQ ID NO:2. In some embodiments, a CBH1a variant of the present invention comprises the entire length of the CBD (optionally with the above-described modifications and optionally with other substitutions and/or modifications as described herein). In some embodiments, the CBH1a variant has a C-terminal deletion of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 residues. In some embodiments, the CBH1a variant has a C-terminal deletion of 1-10, 5-20, or 10-35 residues. In some embodiments, the CBH1a variant comprises a C-terminal deletion as described herein and further comprises one or more non-CBD residues appended to the C-terminus of the variant polypeptide.

In some embodiments, a cellobiohydrolase variant of the present invention comprises a signal peptide sequence at the amino-terminus of the polypeptide. In some embodiments, the signal peptide is an endogenous M. thermophila cellobiohydrolase signal peptide. In some embodiments, the signal peptide is a signal peptide from another M. thermophila secreted protein. In some embodiments, the signal peptide is a signal peptide from a cellobiohydrolase or another secreted protein secreted from an organism other than M. thermophila (e.g., from a filamentous fungus, yeast, or bacteria). In some embodiments the signal peptide is a non-naturally occurring signal peptide.

### III. Substitutions in Homologous Cellobiohydrolases

In another aspect, the present invention provides cellobiohydrolase proteins that are variants of naturally occurring cellobiohydrolases of fungal species other than M. thermophila which comprise a substitution or modification at at least one position corresponding to a position of a substitution or modification of a M. thermophila CBH1a described herein, and which have improved properties, such as improved thermoactivity, improved specific activity, and/or improved thermostability, relative to the cellobiohydrolase from which the variant is derived (e.g., wild-type M. thermophila CBH1a or a CBH1 of a fungal species other than M. thermophila). As used herein, the term “derived from,” as used with reference to a cellobiohydrolase (e.g., a wild-type M. thermophila CBH1a or a CBH1 of a fungal species other than M. thermophila from which a variant is “derived”), refers to a wild-type cellobiohydrolase sequence into which one or more mutations (e.g., amino acid substitutions) have been introduced to result in a cellobiohydrolase variant.

In particular, analogous substitutions may be made in fungal cellobiohydrolases with significant sequence similarity to M. thermophila CBH1a. A number of fungal strains (including but not limited to Thielavia, Humicola, Chaetomium, Neurospora, Chaetomidium, Botryosphaeria, Trichophaea, Aspergillus, Schizophyllum, and Agaricus) express cellobiohydrolases with significant sequence identity to M. thermophila CBH1a.

In some embodiments, a recombinant cellobiohydrolase of the present invention is derived from a fungal protein shown in Table 1.

TABLE 1
Cellobiohydrolase homologs having significant sequence identity to M. thermophila CBH1a

| | | | Protein | |
|---|---|---|---|---|
| | | % identity to | length | SEQ |
| Organism | Accession No. | SEQ ID NO: 2 | (aa)* | ID NO |
| Myceliophthora thermophila | | 100 | 526 | 2 |
| Thielavia australiensis | gi\|29160311\|emb\|CAD79782.1\| | 82 | 538 | 4 |
| Humicola grisea | gi\|950686\|dbj\|BAA09785.1\| | 81 | 525 | 5 |
| Chaetomium thermophilum | gi\|156712282\|emb\|CAM98448.1\| | 79 | 532 | 6 |
| Sordaria macrospora | emb\|CBI55936.1\| | 75 | 522 | 7 |
| Chaetomidium pingtungium | gi\|29160357\|emb\|CAD79795.1\| | 65 | 532 | 8 |
| Botryosphaeria rhodina | gi\|29160339\|emb\|CAD79786.1\| | 65 | 454 | 9 |
| Trichophaea saccata | gi\|29160341\|emb\|CAD79787.1\| | 64 | 458 | 10 |
| Aspergillus nidulans | gi\|40747437\|gb\|EAA66593.1\| | 64 | 526 | 11 |
| Schizophyllum commune | gi\|61743603\|gb\|AAX55505.1\| | 63 | 455 | 12 |
| Agaricus bisporus | gi\|1679595\|emb\|CAA90422.1\| | 61 | 506 | 13 |

It is within the ability of one of ordinary skill in the art to identify other examples of structurally homologous proteins. The present invention provides variants of these and other homologous cellobiohydrolase proteins in which substitutions are made at residues corresponding to those identified herein in the M. thermophila CBH1a protein.

To produce CBH1a homologs with improved properties, the sequences of M. thermophila CBH1a and the cellobiohydrolase homolog (e.g., a homolog listed in Table 1) can be aligned in a pairwise manner as described supra. Based on the alignment, a residue in a position in the homolog that corresponds, based on the alignment, with a specified position in M. thermophila CBH1a is identified.

Thus, in some embodiments, the present invention provides a recombinant cellobiohydrolase variant comprising at least 50% (or at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99%) sequence identity to SEQ ID NO:2 and comprising one or more amino acid substitutions selected from X2H, X23E, X24L/N/V, X25D, X30T, X39D, X46H, X58Y, X60T, X61N, X62L, X63A/D, X64P, X75L, X76V, X77W, X83T/W, X96E/N, X97G, X111Q, X115N, X117D/E, X118D, X119D, X177E, X178S, X208G, X216N, X217E, X221E, X222S, X223A/G/L/W, X250I, X251W, X267E, X268Y, X271V, X275N, X289D/E/R/W, X294Q, X339V, X343D, X349T, X350A, X351Y, X354L, X357V, X362A/P/T, X363N, X367Y, X379E, X392E/P/S, X394A/D, X403Y, X408D/S, X409D, X417S, X424R, X426S, X427N, X428L, X433S, X434E, X451Y, X452E, X453L, X455F/L/Q/W, X456R, X457R, X458A, X459E, X464R, X465A/M/R, X466A/W, X467R, X469C, X470D/E, X472A, X473UT/W, X474D, X475F/L/R/W, X476P, X477V, X478K/W, X479K, X480M, X481F/P/R/W, X482G, X483G, X484L/P, X485W, X486A, X487D/I, X489Y, X490A/W, X491 A/P, X492C/V, X494A/E/K/R/W, X495G, X496L/R/W, X500E/R/V, X501Q/S/W, X502I/L/Q, X504A/G/M, X505E/P, X506A/H/L/V, X507W, X508W, X510R, X511P/W, X512A, X513G/W, X516A/K/R, X517E/G/P, X518A/E, X519H, X521A, X523A/W, X524G/P/W, and X526E/F/P, wherein the position is numbered with reference to SEQ ID NO:2, and wherein the cellobiohydrolase variant has increased thermoactivity or specific activity in comparison to secreted wild-type CBH1a (SEQ ID NO:2).

In some embodiments, the cellobiohydrolase variant has at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a fungal cellobiohydrolase protein and comprises one or more one or more substitutions described herein. In some embodiments, the isolated cellobiohydrolase variant comprises at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% sequence identity to a type 1 cellobiohydrolase from M. thermophila (SEQ ID NO:2), Thielavia australiensis (SEQ ID NO:4), Humicola grisea (SEQ ID NO:5), Chaetomium thermophilum (SEQ ID NO:6), Sordaria macrospora (SEQ ID NO:7), Chaetomidium pingtungium (SEQ ID NO:8), Botryosphaeria rhodina (SEQ ID NO:9), Trichophaea saccata (SEQ ID NO:10), Aspergillus nidulans (SEQ ID NO:11), Schizophyllum commune (SEQ ID NO:12), or Agaricus bisporus (SEQ ID NO:13). In some embodiments, the cellobiohydrolase variant has at least 95% sequence identity to M. thermophila (SEQ ID NO:2), Thielavia australiensis (SEQ ID NO:4), Humicola grisea (SEQ ID NO:5), Chaetomium thermophilum (SEQ ID NO:6), Sordaria macrospora (SEQ ID NO:7), Chaetomidium pingtungium (SEQ ID NO:8), Botryosphaeria rhodina (SEQ ID NO:9), Trichophaea saccata (SEQ ID NO:10), Aspergillus nidulans (SEQ ID NO:11), Schizophyllum commune (SEQ ID NO:12), or Agaricus bisporus (SEQ ID NO:13) and comprises one or more substitutions described herein (e.g., one or more amino acid substitutions or one or more substitution sets listed in Table 2).

In some embodiments, the variant has increased improved thermoactivity, improved specific activity, and/or improved thermostability in comparison to wild-type M. thermophila CBH1a (SEQ ID NO:2). In some embodiments, the variant has increased improved thermoactivity, improved specific activity, and/or improved thermostability in comparison to the cellobiohydrolase polypeptide (e.g., a cellobiohydrolase polypeptide from a fungal strain, e.g., a cellobiohydrolase polypeptide from Agaricus, Aspergillus, Chaetomium, Chrysosporium, Humicola, Schizophyllum, Sordaria, Thielavia, or Trichophaea) from which the variant is derived.

In some embodiments, the present invention relates to a method of making CBH1a variants having improved thermoactivity, improved specific activity, and/or improved thermostability. In some embodiments, the method comprises:

(a) identifying a sequence that comprises at least about 70% (or at least about 75%, at least about 80%, at least about 85%, at least about 90%, at least about 95%, at least about 96%, at least about 97%, at least about 98%, or at least about 99%) sequence identity to SEQ ID NO:2;

(b) aligning the identified sequence with the sequence of SEQ ID NO:2; and

(c) making a mutation at one or more positions selected from positions corresponding to Y2, L23, T24, A25, S30, G39, Q46, T58, R60, T61, S62, S63, A64, T75, S76, Y77, S83, S96, S97, K111, K115, Q117, Y118, S119, S177, G178, E208, D216, A217, T221, G222, K223, V250, I251, T267, D268, A271, D275, K289, K294, I339, E343, Q349, D350, W351, R354, A357, V362, T363, D367, A379, H392, V394, W403, A408, G409, A417, V424, A426, E427, V428, P433, N434, V451, S452, G453, P455, D456, G457, G458, S459, P464, P465, V466, S467, S469, T470, V472, P473, S474, S475, S476, T477, T478, S479, S480, G481, S482, S483, G484, P485, T486, G487, T489, G490, V491, A492, H494, Y495, E496, G500, I501, G502, T504, G505, P506, T507, Q508, T510, S511, P512, Y513, T516, K517, L518, N519, W521, S523, Q524, and L526.

In some embodiments, step (c) of the method comprises making one or more amino acid substitutions selected from Y2H, L23E, T24L/N/V, A25D, 530T, G39D, Q46H, T58Y, R60T, T61N, S62L, S63A/D, A64P, T75L, S76V, Y77W, S83T/W, S96E/N, S97G, K111Q, K115N, Q117D/E, Y118D, 5119D, S177E, G178S, E208G, D216N, A217E, T221E, G222S, K223A/G/L/W, V2501,1251W, T267E, D268Y, A271V, D275N, K289D/E/R/W, K294Q, I339V, E343D, Q349T, D350A, W351Y, R354L, A357V, V362A/P/T, T363N, D367Y, A379E, H392E/P/S, V394A/D, W403Y, A408D/S, G409D, A4175, V424R, A426S, E427N, V428L, P433S, N434E, V451Y, S452E, G453L, P455F/L/Q/W, D456R, G457R, G458A, S459E, P464R, P465A/M/R, V466A/W, S467R, S469C, T470D/E, V472A, P473L/T/W, S474D, S475F/L/R/W, S476P, T477V, T478K/W, S479K, S480M, G481F/P/R/W, S482G, S483G, G484L/P, P485W, T486A, G487D/I, T489Y, G490A/W, V491A/P, A492C/V, H494A/E/K/R/W, Y495G, E496L/R/W, G500E/R/V, I501Q/S/W, G502I/L/Q, T504A/G/M, G505E/P, P506A/H/L/V, I507W, Q508W, T510R, S511P/W, P512A, Y513G/W, T516A/K/R, K517E/G/P, L518A/E, N519H, W521A, S523A/W, Q524G/P/W, and L526E/F/P.

In some embodiments, the method further comprises determining whether the one or more amino acid substitutions increase the thermoactivity and/or specific activity of the cellobiohydrolase variant in comparison to secreted wild-type M. thermophila CBH1a (SEQ ID NO:2).

### IV. Generation of Cellobiohydrolase Variants

A cellobiohydrolase variant polypeptide of the invention can be subject to further modification to generate new polypeptides that retain the specific substitutions that characterize the variant and which may have desirable properties. For example, a polynucleotide encoding a cellobiohydrolase with an improved property can be subjected to additional rounds of mutagenesis treatments to generate polypeptides with further improvements in the desired enzyme or enzyme properties.

Given the wild-type M. thermophila CBH1a sequence or the sequence of a wild-type fungal homolog of M. thermophila CBH1a, cellobiohydrolase variants can be generated according to the methods described herein and can be screened for the presence of improved properties, such as improved thermoactivity, improved specific activity, and/or improved thermostability. Libraries of cellobiohydrolase variant polypeptides and/or polynucleotides encoding the variants may be generated from a parental sequence (e.g., wild-type M. thermophila CBH1a, or a wild-type cellobiohydrolase from another fungal strain such as a cellobiohydrolase of Table 1, or one of the cellobiohydrolase variants exemplified herein), and screened using a high throughput screen to determine improved properties such as improved thermoactivity, improved specific activity, and/or improved thermostability at desired conditions, as described herein. Mutagenesis and directed evolution methods are well known in the art and can be readily applied to polynucleotides encoding cellobiohydrolase variants exemplified herein to generate variant libraries that can be expressed, screened, and assayed using the methods described herein. See, e.g., Ling, et al., 1999, “Approaches to DNA mutagenesis: an overview,” Anal. Biochem., 254(2):157-78; Dale, et al., 1996, “Oligonucleotide-directed random mutagenesis using the phosphorothioate method,” Methods Mol. Biol., 57:369-74; Smith, 1985, “In vitro mutagenesis,” Ann. Rev. Genet., 19:423-462; Botstein, et al., 1985, “Strategies and applications of in vitro mutagenesis,” Science, 229:1193-1201; Carter, 1986, “Site-directed mutagenesis,” Biochem. J., 237:1-7; Kramer, et al., 1984, “Point Mismatch Repair,” Cell, 38:879-887; Wells, et al., 1985, “Cassette mutagenesis: an efficient method for generation of multiple mutations at defined sites,” Gene, 34:315-323; Minshull, et al., 1999, “Protein evolution by molecular breeding,” Current Opinion in Chemical Biology, 3:284-290; Christians, et al., 1999, “Directed evolution of thymidine kinase for AZT phosphorylation using DNA family shuffling,” Nature Biotechnology, 17:259-264; Crameri, et al., 1998, “DNA shuffling of a family of genes from diverse species accelerates directed evolution,” Nature, 391:288-291; Crameri, et al., 1997, “Molecular evolution of an arsenate detoxification pathway by DNA shuffling,” Nature Biotechnology, 15:436-438; Zhang, et al., 1997 “Directed evolution of an effective fucosidase from a galactosidase by DNA shuffling and screening,” Proceedings of the National Academy of Sciences, U.S.A., 94:45-4-4509; Crameri, et al., 1996, “Improved green fluorescent protein by molecular evolution using DNA shuffling,” Nature Biotechnology, 14:315-319; Stemmer, 1994, “Rapid evolution of a protein in vitro by DNA shuffling,” Nature, 370:389-391; Stemmer, 1994, “DNA shuffling by random fragmentation and reassembly: In vitro recombination for molecular evolution,” Proceedings of the National Academy of Sciences, U.S.A., 91:10747-10751; WO 95/22625; WO 97/0078; WO 97/35966; WO 98/27230; WO 00/42651; and WO 01/75767, all of which are incorporated herein by reference.

Cellobiohydrolase variants having the amino acid substitutions described herein can also be synthetically generated. Chemically synthesized polypeptides may be generated using the well-known techniques of solid phase, liquid phase, or peptide condensation techniques, and can include any combination of amino acids as desired to produce the variants described herein. Synthetic amino acids can be obtained from Sigma, Cambridge Research Biochemical, or any other chemical company familiar to those skilled in the art.

In generating variants that comprise substitutions, insertions or deletions at positions in addition to those described supra, the ordinarily skilled practitioner will be aware that certain regions of the cellobiohydrolase protein are less tolerant than others to substitutions (especially non-conservative substitutions). Thus, in some embodiments, variant cellobiohydrolase polypeptides retain conserved residues and functional domains from the parent.

For example, cellobiohydrolase proteins are characterized by the presence of several functional domains, including a catalytic domain and a cellulose binding domain. The catalytic and cellulose binding domain structures are known in the art for a number of cellobiohydrolase proteins. For example, the CBH1a of SEQ ID NO:2 comprises a catalytic domain at amino acids 1-437 and a cellulose binding domain at amino acids 474-509. Those of skill in the art are aware of structure-function relationships for cellobiohydrolase protein domains. See, e.g., Abuja et al., Eur Biophys J 15:339-342 (1988); Reinikainen et al., Proteins 14:475-482 (1992). In some embodiments, amino acid substitutions are made to a greater extent outside a functional domain (e.g., the catalytic domain and/or cellulose binding domain) than within the functional domain. In some embodiments, a cellobiohydrolase variant comprises relatively few substitutions outside of the catalytic domain and/or cellulose binding domain other than those amino acid substitions described herein (e.g., in Table 2).

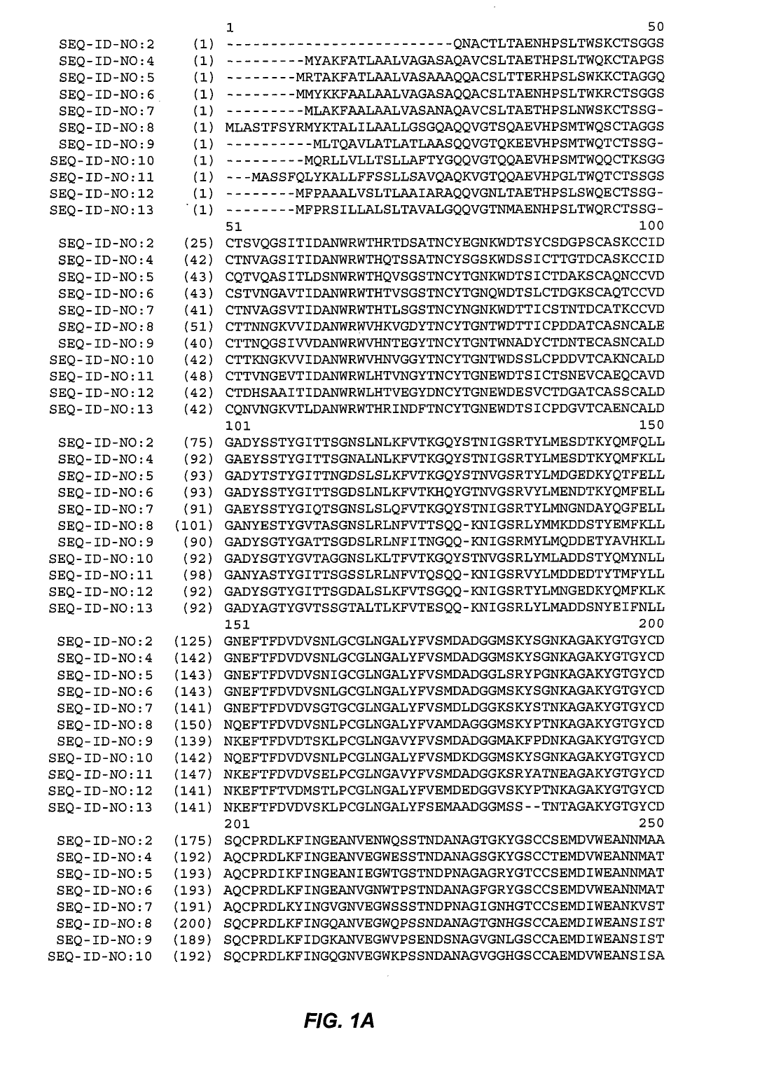

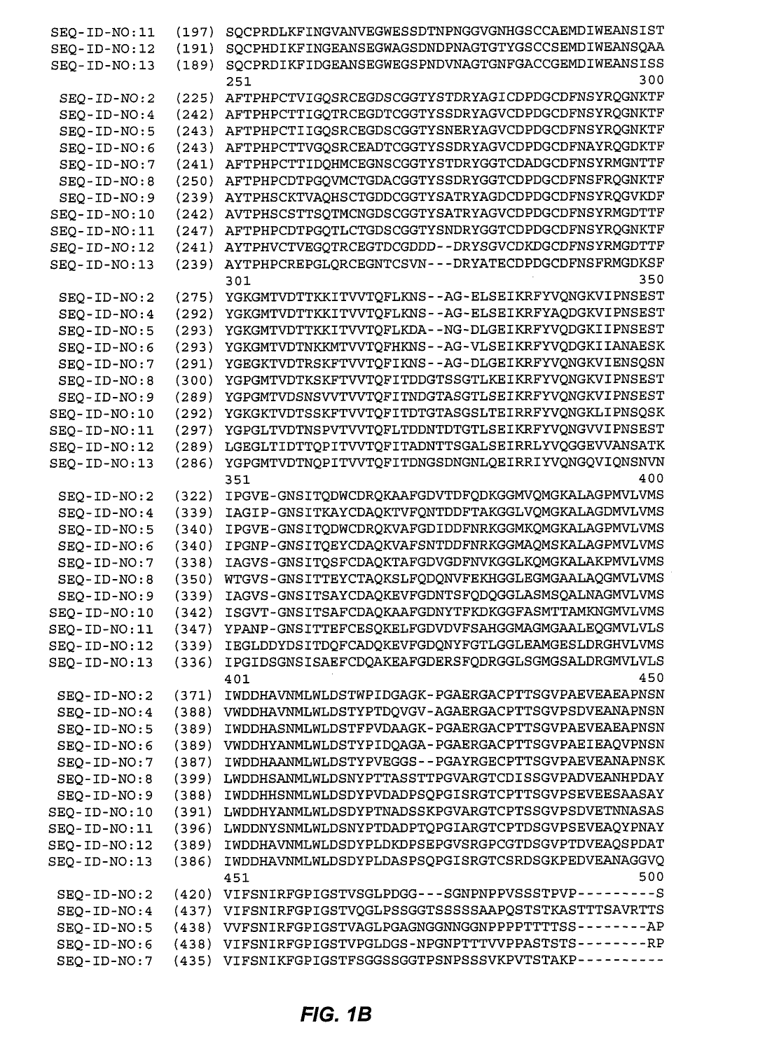

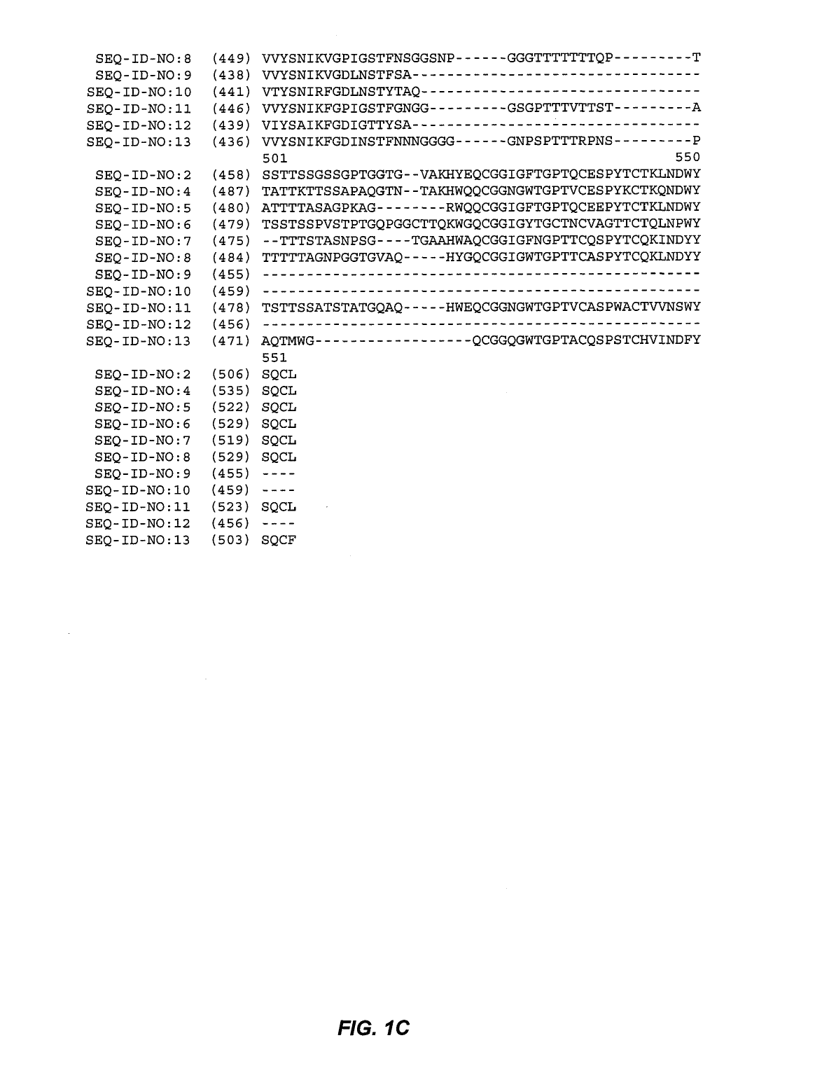

Other regions of the cellobiohydrolase protein that may be less tolerant than others to substitutions may be identified by amino acid sequence alignment between two or more cellobiohydrolase proteins (e.g., an amino acid sequence alignment as shown in FIG. 1). Using the alignment one of skill in the art can compare the sequences and identify residues or regions that are conserved between the cellobiohydrolase proteins). One of skill in the art will recognize that amino acid residues that are highly conserved among multiple cellobiohydrolase proteins may be less tolerant of substitutions than residues which are not conserved among multiple cellobiohydrolase proteins.

### V. Cellobiohydrolase Thermoactivity and Thermostability

Cellobiohydrolase activity and thermostability can be determined by methods described in the Examples section (e.g., Examples 2, 4, and 5), and/or using any other methods known in the art. For example, cellobiohydrolase activity may be determined using a 4-methylumbelliferyl β-D-lactopyranoside (MUL) assay, or using an assay that measures the conversion of crystalline cellulose to glucose. Assays for determining cellobiohydrolase activity by measuring the production of other sugars are also known in the art.

Cellobiohydrolase activity can be determined, for example, using a cellulose assay, in which the ability of the cellobiohydrolase variants to hydrolyze a cellulose substrate to cellobiose (e.g., crystalline cellulose or wheat straw pretreated under acidic conditions) under specific temperature and/or pH conditions is measured using a β-glucosidase to convert the cellobiose to glucose. For example, a supernatant containing the secreted cellobiohydrolase is mixed with a cellulose substrate, e.g., wheat straw pretreated under acidic conditions, or crystalline cellulose (e.g., Avicel®), in a buffered solution and β-glucosidase is added to convert the cellobiose to glucose. A biotransformation reaction is performed under specific time, temperature, and/or pH conditions, for example, pH 5.0, 55° C., for 24-72 hours. An aliquot of the biotransformation reaction is then assayed for conversion of cellulose (e.g., wheat straw pretreated under acidic conditions or crystalline cellulose) to fermentable soluble sugars (e.g., glucose). Conversion of cellulose substrate (e.g., wheat straw pretreated under acidic conditions or crystalline cellulose) to fermentable sugar oligomers (e.g., glucose) can be determined by art-known means, including but not limited to coupled enzymatic assay and colorimetric assay. For example, glucose concentrations can be determined using a coupled enzymatic assay based on glucose oxidase and horseradish peroxidase (e.g., GOPOD assay) as exemplified in Trinder, P. (1969) Ann. Clin. Biochem. 6:24-27, which is incorporated herein by reference in its entirety. GOPOD assay kits are known in the art and are readily commercially available, e.g., from Megazyme (Wicklow, Ireland). Methods for performing GOPOD assays are known in the art; see, e.g., McCleary et al., J. OAC Int 85(5):1103-11 (2002), the contents of which are incorporated by reference herein. Additional methods of cellobiose quantification include chromatographic methods, for example by HPLC as exemplified in the incorporated materials of U.S. Pat. Nos. 6,090,595 and 7,419,809.

In one exemplary assay, biotransformation reactions are performed by mixing 250-120 μL of supernatant into a reaction mixture containing 10 mg biomass (wheat straw pretreated under acidic conditions), 180-360 μL filtrate (soluble fraction of pretreated wheat straw obtained by washing pretreated substrate solids with water) and 5 mg/L β-glucosidase, which converts cellobiose to glucose, in 128 mM sodium acetate buffer pH 5 for a total volume of 500 μL. Biotransformation is performed at pH 5, 55° C. for an appropriate amount of time. The amount of glucose produced is then determined by art-known means, for example GOPOD assay. For the GOPOD assay, fermentable sugar oligomer (e.g., glucose) production is measured by mixing 20 μL of the above reaction with 180 μL of GOPOD assay mix. The reactions are allowed to incubate for 30 min at room temperature. Absorbance of the solution is measured at 510 nm to determine the amount of glucose produced in the original biotransformation reaction, which is used to calculate cellobiohydrolase activity.

In another exemplary assay, biotransformation reactions are performed by mixing 60 μl clear supernatant with 40 μl of a slurry of crystalline cellulose in 340 mM sodium acetate buffer pH 4.2-5.0 (final concentration: 200 g/L crystalline cellulose; a glass bead/well). Additionally, 50 μl of beta-glucosidase supernatant is added to the reaction mixture for the conversion of cellobiose to glucose. Biotransformation is performed at pH 4.5, 65-70° C. for an appropriate amount of time. Glucose generation is measured using a GOPOD assay. For the GOPOD assay, fermentable sugar oligomer (e.g., glucose) production is measured by mixing 10 μl of the above reaction with 190 μl of GOPOD assay mix. The reactions are allowed to shake for 30 min at room temperature. Absorbance of the solution is measured at 510 nm to determine the amount of glucose produced in the original biotransformation reaction. The amount of glucose produced is measured at 510 nm to calculate cellobiohydrolase activity.

Cellobiohydrolase thermostability can be determined, for example, by exposing the cellobiohydrolase variants and the reference (e.g., wild-type) cellobiohydrolase to stress conditions of elevated temperature and/or low pH for a desired period of time and then determining residual cellobiohydrolase activity using an assay that measures the conversion of cellulose to glucose. For example, a supernatant containing the secreted cellobiohydrolase is exposed to stress conditions, for example, pH 4.5-5.0 and temperature 55-60° C. for 1 hour. After the challenge with stress conditions, residual cellobiohydrolase activity is measured by performing a biotransformation reaction on crystalline cellulose substrate such as Avicel in the presence of excess β-glucosidase at pH 5, 55° C. for 18 hours. The conversion of cellulose substrate (e.g., crystalline cellulose) to fermentable sugar oligomers (e.g., glucose) can be determined by art-known means, for example using any of the assays as described above, such as coupled enzymatic assay based on glucose oxidase and horseradish peroxidase (e.g., GOPOD) assay.

In an exemplary assay, thermostability is screened using a cellulose-based High Throughput Assay. In deep, 96-well microtiter plates 100 μL of media supernatant containing cellobiohydrolase variant is added to 300 μL of 200 mM sodium acetate buffer pH 5.0 containing 0.15 g/L f3-glucosidase and 75 g/L crystalline cellulose. After sealing with aluminum/polypropylene laminate heat seal tape (Velocity 11 (Menlo Park, Calif.), Cat#06643-001)), the plates are shaken at 55° C. for 16-24 hours. The plates are centrifuged at 4000 rpm for 5 minutes. The glucose produced is then is measured by any art-known means, for example using any of the assays as described above, such as coupled enzymatic assay based on glucose oxidase and horseradish peroxidase (e.g., GOPOD) assay.

Some cellobiohydrolase variants of the invention will have improved thermoactivity as compared to a reference sequence (e.g., a wild-type cellobiohydrolase or another cellobiohydrolase variant). In some embodiments, a cellobiohydrolase variant exhibits cellobiohydrolase thermoactivity that is at least about 1-fold, at least about 1.1-fold, at least about 1.2-fold, at least about 1.3-fold, at least about 1.4-fold, or at least about 1.5-fold or greater than the cellobiohydrolase thermoactivity of a reference cellobiohydrolase (e.g., the wild-type cellobiohydrolase of SEQ ID NO:2) when tested under the same conditions. In some embodiments, the thermoactivity of a cellobiohydrolase variant of the invention at pH 5 and 55° C. is at least about 1-fold, at least about 1.1-fold, at least about 1.2-fold, at least about 1.3-fold, at least about 1.4-fold, or at least about 1.5-fold or greater than the cellobiohydrolase thermoactivity of a reference cellobiohydrolase (e.g., the wild-type cellobiohydrolase of SEQ ID NO:2) under the same conditions.

### VI. Fusion Peptides and Additional Sequence Elements

In some embodiments, a cellobiohydrolase variant of the present invention further comprises additional sequences which do not alter the encoded activity of the cellobiohydrolase. For example, the cellobiohydrolase may be linked to an epitope tag or to another sequence useful in purification.

The present invention also provides cellobiohydrolase variant fusion polypeptides, wherein the fusion polypeptide comprises an amino acid sequence encoding a cellobiohydrolase variant polypeptide of the present invention or fragment thereof, linked either directly or indirectly through the N- or C-terminus of the cellobiohydrolase variant polypeptide to an amino acid sequence encoding at least a second (additional) polypeptide. The cellobiohydrolase variant fusion polypeptide may further include amino acid sequence encoding a third, fourth, fifth, or additional polypeptides. Typically, each additional polypeptide has a biological activity, or alternatively, is a portion of a polypeptide that has a biological activity, where the portion has the effect of improving expression and/or secretion and/or purification and/or detection of the fusion polypeptide from the desired expression host. These sequences may be fused, either directly or indirectly, to the N- or C-terminus of the cellobiohydrolase variant polypeptide or fragment thereof, or alternatively, to the N- or C-terminus of the additional polypeptides having biological activity.

In some embodiments, the additional polypeptide(s) encode an enzyme or active fragment thereof, and/or a polypeptide that improves expression and/or secretion of the fusion polypeptide from the desired expression host cell. For example, the additional polypeptide may encode a cellulase (for example, a cellobiohydrolase having a different amino acid sequence from the cellobiohydrolase variant polypeptide in the fusion polypeptide, or a polypeptide exhibiting endoglucanase activity or β-glucosidase activity) and/or a polypeptide that improves expression and secretion from the desired host cell, such as, for example, a polypeptide that is normally expressed and secreted from the desired expression host, such as a secreted polypeptide normally expressed from filamentous fungi. These include glucoamylase, α-amylase and aspartyl proteases from Aspergillus niger, Aspergillus niger var awamori, and Aspergillus oryzae, cellobiohydrolase I, cellobiohydrolase II, endoglucanase I, and endoglucase III from Trichoderma and glucoamylase from Neurospora and Humicola species. See WO 98/31821, which is incorporated herein by reference.

The polypeptide components of the fusion polypeptide may be linked to each other indirectly via a linker. Linkers suitable for use in the practice of the present invention are described in WO 2007/075899, which is incorporated herein by reference. Exemplary linkers include peptide linkers of from 1 to about 40 amino acid residues in length, including those from about 1 to about 20 amino acid residues in length, and those from about 1 to about 10 amino acid residues in length. In some embodiments, the linkers may be made up of a single amino acid residue, such as, for example, a Gly, Ser, Ala, or Thr residue or combinations thereof, particularly Gly and Ser. Linkers employed in the practice of the present invention may be cleavable. Suitable cleavable linkers may contain a cleavage site, such as a protease recognition site. Exemplary protease recognition sites are well known in the art and include, for example, Lys-Arg (the KEX2 protease recognition site, which can be cleaved by a native Aspergillus KEX2-like protease), Lys and Arg (the trypsin protease recognition sites). See, for example, WO 2007/075899, which is incorporated herein by reference.

### Signal Peptides

In some embodiments, the cellobiohydrolase variant polypeptides of the present invention are secreted from the host cell in which they are expressed (e.g., a yeast or fungal cell) and are expressed as a pre-protein including a signal peptide, i.e., an amino acid sequence linked to the amino terminus of a polypeptide and which directs the encoded polypeptide into the cell secretory pathway. In one embodiment, the signal peptide is an endogenous M. thermophila cellobiohydrolase signal peptide, e.g., the signal peptide of a CBH1a pre-protein having the sequence MYAKFATLAALVAGAAA (SEQ ID NO:3). In other embodiments, signal peptides from other M. thermophila secreted proteins are used.

Still other signal peptides may be used, depending on the host cell and other factors. Effective signal peptide coding regions for filamentous fungal host cells include, but are not limited to, the signal peptide coding regions obtained from Aspergillus oryzae TAKA amylase, Aspergillus niger neutral amylase, Aspergillus niger glucoamylase, Rhizomucor miehei asparatic proteinase, Humicola insolens cellulase, Humicola lanuginosa lipase, and T. reesei cellobiohydrolase II (TrCBH2).

Effective signal peptide coding regions for bacterial host cells are the signal peptide coding regions obtained from the genes for Bacillus NCIB 11837 maltogenic amylase, Bacillus stearothermophilus alpha-amylase, Bacillus licheniformis subtilisin, Bacillus licheniformis-lactamase, Bacillus stearothermophilus neutral proteases (nprT, nprS, nprM), and Bacillus subtilis prsA. Further signal peptides are described by Simonen and Palva, 1993, Microbiol Rev 57: 109-137 (incorporated herein by reference).

Useful signal peptides for yeast host cells also include those from the genes for Saccharomyces cerevisiae alpha-factor, Saccharomyces cerevisiae SUC2 invertase (see Taussig and Carlson, 1983, Nucleic Acids Res 11:1943-54; SwissProt Accession No. P00724), and others. See, e.g., Romanos et al., 1992, Yeast 8:423-488. Variants of these signal peptides and other signal peptides are suitable.

### Cellulose Binding Domains

A cellulose binding domain (CBD) is a domain in cellulose degrading enzymes that binds specifically to cellulose. Some cellobiohydrolases and other cellulases, including wild-type M. thermophila CBH1a, have a multidomain structure comprising a catalytic domain and a CBD joined by a linker peptide. For example, the CBH1a of SEQ ID NO:2 comprises a catalytic domain at amino acids 1-437 and a CBD at amino acids 474-509. In some embodiments, a cellobiohydrolase variant of the present invention comprises a CBD. CBDs may be homologous or heterologous to the catalytic domain. A homologous CBD is associated in the wild-type cellobiohydrolase with the parental catalytic domain. For example, in wild-type M. thermophila CBH1a the CBD is homologous to the catalytic domain.

In some embodiments, a cellobiohydrolase variant of the present invention has multiple CBDs. The multiple CBDs can be in tandem or in different regions of the polypeptide.

In some embodiments, a cellobiohydrolase variant of the present invention lacks a CBD. For example, in some embodiments the CBD of the cellobiohydrolase is cleaved from the catalytic domain following secretion of the enzyme. Alternatively, engineered cellobiohydrolases lacking a CBD may be used.

### VII. Polynucleotides and Expression Systems Encoding Cellobiohydrolase Variants

In another aspect, the present invention provides polynucleotides encoding the variant cellobiohydrolase polypeptides as described herein. The polynucleotide may be operably linked to one or more heterologous regulatory or control sequences that control gene expression to create a recombinant polynucleotide capable of expressing the polypeptide. Expression constructs containing a heterologous polynucleotide encoding the engineered cellobiohydrolase can be introduced into appropriate host cells to express the cellobiohydrolase.

In some embodiments, the cellobiohydrolase variant is generated from a wild-type cellobiohydrolase cDNA sequence (e.g., a wild-type M. thermophila CBH1a cDNA sequence (SEQ ID NO:1), or a cDNA sequence encoding a wild-type protein of Table 1) or the portion thereof comprising the open reading frame, with changes made as required at the codons corresponding to substitutions described herein (e.g., at Table 2). In addition, one or more “silent” nucleotide changes can be incorporated. A DNA sequence may also be designed for high codon usage bias codons (codons that are used at higher frequency in the protein coding regions than other codons that code for the same amino acid). The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. A codon whose frequency increases with the level of gene expression is typically an optimal codon for expression. In particular, a DNA sequence can be optimized for expression in a particular host organism. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; Codon W, John Peden, University of Nottingham; McInerney, J. O, 1998, Bioinformatics 14:372-73; Stenico et al., 1994, Nucleic Acids Res. 222437-46; Wright, F., 1990, Gene 87:23-29; Wada et al., 1992, Nucleic Acids Res. 20:2111-2118; Nakamura et al., 2000, Nucl. Acids Res. 28:292; Henaut and Danchin, “Escherichia coli and Salmonella,” 1996, Neidhardt, et al. Eds., ASM Press, Washington D.C., p. 2047-2066, all of which are incorporated herein be reference). The data source for obtaining codon usage may rely on any available nucleotide sequence capable of coding for a protein, e.g., complete protein coding sequences (CDSs), expressed sequence tags (ESTs), or predicted coding regions of genomic sequences.

Those having ordinary skill in the art will understand that due to the degeneracy of the genetic code, a multitude of nucleotide sequences encoding cellobiohydrolase polypeptides of the present invention exist. For example, the codons AGA, AGG, CGA, CGC, CGG, and CGU all encode the amino acid arginine. Thus, at every position in the nucleic acids of the invention where an arginine is specified by a codon, the codon can be altered to any of the corresponding codons described above without altering the encoded polypeptide. It is understood that U in an RNA sequence corresponds to T in a DNA sequence. The invention contemplates and provides each and every possible variation of nucleic acid sequence encoding a polypeptide of the invention that could be made by selecting combinations based on possible codon choices.

Polynucleotides encoding cellobiohydrolases can be prepared using methods that are well known in the art. Typically, oligonucleotides of up to about 40 bases are individually synthesized, then joined (e.g., by enzymatic or chemical ligation methods, or polymerase-mediated methods) to form essentially any desired continuous sequence. For example, polynucleotides of the present invention can be prepared by chemical synthesis using, for example, the classical phosphoramidite method described by Beaucage, et al., 1981, Tetrahedron Letters, 22:1859-69, or the method described by Matthes, et al., 1984, EMBO J. 3:801-05, both of which are incorporated herein by reference. These methods are typically practiced in automated synthetic methods. According to the phosphoramidite method, oligonucleotides are synthesized, e.g., in an automatic DNA synthesizer, purified, annealed, ligated and cloned in appropriate vectors.

General texts that describe molecular biological techniques which are useful herein, including the use of vectors, promoters, protocols sufficient to direct persons of skill through in vitro amplification methods, including the polymerase chain reaction (PCR) and the ligase chain reaction (LCR), and many other relevant methods, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Ausubel, supra, all of which are incorporated herein by reference. Reference is made to Berger, Sambrook, and Ausubel, as well as Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87, 1874; Lomeli et al. (1989) J. Clin. Chem. 35, 1826; Landegren et al., (1988) Science 241, 1077-1080; Van Brunt (1990) Biotechnology 8, 291-294; Wu and Wallace, (1989) Gene 4, 560; Barringer et al. (1990) Gene 89, 117, and Sooknanan and Malek (1995) Biotechnology 13: 563-564, all of which are incorporated herein by reference. Methods for cloning in vitro amplified nucleic acids are described in Wallace et al., U.S. Pat. No. 5,426,039, which is incorporated herein by reference.

### Vectors

The present invention makes use of recombinant constructs comprising a sequence encoding a cellobiohydrolase as described above. In a particular aspect the present invention provides an expression vector comprising a cellobiohydrolase polynucleotide operably linked to a heterologous promoter. Expression vectors of the present invention may be used to transform an appropriate host cell to permit the host to express the cellobiohydrolase protein. Methods for recombinant expression of proteins in fungi and other organisms are well known in the art, and a number expression vectors are available or can be constructed using routine methods. See, e.g., Tkacz and Lange, 2004, ADVANCES IN FUNGAL BIOTECHNOLOGY FOR INDUSTRY, AGRICULTURE, AND MEDICINE, KLUWER ACADEMIC/PLENUM PUBLISHERS. New York; Zhu et al., 2009, Construction of two Gateway vectors for gene expression in fungi Plasmid 6:128-33; Kavanagh, K. 2005, FUNGI: BIOLOGY AND APPLICATIONS Wiley, all of which are incorporated herein by reference.

Nucleic acid constructs of the present invention comprise a vector, such as, a plasmid, a cosmid, a phage, a virus, a bacterial artificial chromosome (BAC), a yeast artificial chromosome (YAC), and the like, into which a nucleic acid sequence of the invention has been inserted. Polynucleotides of the present invention can be incorporated into any one of a variety of expression vectors suitable for expressing a polypeptide. Suitable vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, pseudorabies, adenovirus, adeno-associated virus, retroviruses and many others. Any vector that transduces genetic material into a cell, and, if replication is desired, which is replicable and viable in the relevant host can be used.

In some embodiments, the construct further comprises regulatory sequences, including, for example, a promoter, operably linked to the protein encoding sequence. Large numbers of suitable vectors and promoters are known to those of skill in the art.

### Promoters

In order to obtain high levels of expression in a particular host it is often useful to express the cellobiohydrolase variant of the present invention under the control of a heterologous promoter. A promoter sequence may be operably linked to the 5′ region of the cellobiohydrolase coding sequence using routine methods.

Examples of useful promoters for expression of cellobiohydrolases include promoters from fungi. In some embodiments, a promoter sequence that drives expression of a gene other than a cellobiohydrolase gene in a fungal strain may be used. As a non-limiting example, a fungal promoter from a gene encoding an endoglucanase may be used. In some embodiments, a promoter sequence that drives the expression of a cellobiohydrolase gene in a fungal strain other than the fungal strain from which the cellobiohydrolase variant was derived may be used. As a non-limiting example, if the cellobiohydrolase variant is derived from M. thermophila, a promoter from a T. reesei cellobiohydrolase gene may be used or a promoter as described in WO2010/107303, such as but not limited to the sequences identified as SEQ ID NO:25, SEQ ID NO:26, SEQ ID NO:27, SEQ ID NO:28, or SEQ ID NO:29 in WO2010/107303.

Examples of other suitable promoters useful for directing the transcription of the nucleotide constructs of the present invention in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (WO 96/00787, which is incorporated herein by reference), as well as the NA2-tpi promoter (a hybrid of the promoters from the genes for Aspergillus niger neutral alpha-amylase and Aspergillus oryzae triose phosphate isomerase), promoters such as cbh1, cbh2, egl1, egl2, pepA, hfb1, hfb2, xyn1, amy, and glaA (Nun berg et al., 1984, Mol. Cell Biol., 4:2306-2315, Boel et al., 1984, EMBO J. 3:1581-85 and EPA 137280, all of which are incorporated herein by reference), and mutant, truncated, and hybrid promoters thereof. In a yeast host, useful promoters can be from the genes for Saccharomyces cerevisiae enolase (eno-1), Saccharomyces cerevisiae galactokinase (gal1), Saccharomyces cerevisiae alcohol dehydrogenase/glyceraldehyde-3-phosphate dehydrogenase (ADH2/GAP), and S. cerevisiae 3-phosphoglycerate kinase. Other useful promoters for yeast host cells are described by Romanos et al., 1992, Yeast 8:423-488, incorporated herein by reference. Promoters associated with chitinase production in fungi may be used. See, e.g., Blaiseau and Lafay, 1992, Gene 120243-248 (filamentous fungus Aphanocladium album); Limon et al., 1995, Curr. Genet, 28:478-83 (Trichoderma harzianum), both of which are incorporated herein by reference.

Promoters known to control expression of genes in prokaryotic or eukaryotic cells or their viruses and which can be used in some embodiments of the invention include SV40 promoter, E. coli lac or trp promoter, phage lambda P_{L} promoter, tac promoter, T7 promoter, and the like. In bacterial host cells, suitable promoters include the promoters obtained from the E. coli lac operon, Streptomyces coelicolor agarase gene (dagA), Bacillus subtilis levansucranse gene (sacB), Bacillus licheniformis α-amylase gene (amyl), Bacillus stearothermophilus maltogenic amylase gene (amyM), Bacillus amyloliquefaciens α-amylase gene (amyQ), Bacillus subtilis xyIA and xyIB genes and prokaryotic β-lactamase gene.

Any other promoter sequence that drives expression in a suitable host cell may be used. Suitable promoter sequences can be identified using well known methods. In one approach, a putative promoter sequence is linked 5′ to a sequence encoding a reporter protein, the construct is transfected into the host cell (e.g., M. thermophila) and the level of expression of the reporter is measured. Expression of the reporter can be determined by measuring, for example, mRNA levels of the reporter sequence, an enzymatic activity of the reporter protein, or the amount of reporter protein produced. For example, promoter activity may be determined by using the green fluorescent protein as coding sequence (Henriksen et al, 1999, Microbiology 145:729-34, incorporated herein by reference) or a lacZ reporter gene (Punt et al, 1997, Gene, 197:189-93, incorporated herein by reference). Functional promoters may be derived from naturally occurring promoter sequences by directed evolution methods. See, e.g. Wright et al., 2005, Human Gene Therapy, 16:881-892, incorporated herein by reference.

### Other Expression Elements

Cloned cellobiohydrolases may also have a suitable transcription terminator sequence, a sequence recognized by a host cell to terminate transcription. The terminator sequence is operably linked to the 3′ terminus of the nucleic acid sequence encoding the polypeptide. Any terminator that is functional in the host cell of choice may be used in the present invention.

For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary transcription terminators are described in U.S. Pat. No. 7,399,627, incorporated herein by reference.

Exemplary terminators for yeast host cells can be obtained from the genes for Saccharomyces cerevisiae enolase, Saccharomyces cerevisiae cytochrome C(CYCI), and Saccharomyces cerevisiae glyceraldehyde-3-phosphate dehydrogenase. Other useful terminators for yeast host cells are described by Romanos et al., 1992, Yeast 8:423-88.

A suitable leader sequence may be part of a cloned cellobiohydrolase sequence, which is a nontranslated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

Sequences may also contain a polyadenylation sequence, which is a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells can be from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are described by Guo and Sherman, Mol Cell Bio 15:5983-5990 (1995).

The expression vector of the present invention optionally contains one or more selectable markers, which permit easy selection of transformed cells. A selectable marker is a gene, the product of which provides for biocide or viral resistance, resistance to heavy metals, prototrophy to auxotrophs, and the like. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Embodiments for use in an Aspergillus cell include the amdS and pyrG genes of Aspergillus nidulans or Aspergillus oryzae and the bar gene of Streptomyces hygroscopicus. Suitable markers for yeast host cells are ADE2, HIS3, LEU2, LYS2, MET3, TRP1, and URA3.

### VIII. Host Cells Comprising Cellobiohydrolase Variants

A vector comprising a sequence encoding a cellobiohydrolase is transformed into a host cell in order to allow propagation of the vector and expression of the cellobiohydrolase. In some embodiments, the cellobiohydrolase is post-translationally modified to remove the signal peptide and in some cases may be cleaved after secretion.

The transformed or transfected host cell described above is cultured in a suitable nutrient medium under conditions permitting the expression of the cellobiohydrolase. The medium used to culture the cells may be any conventional medium suitable for growing the host cells, such as minimal or complex media containing appropriate supplements. Cells are optionally grown in HTP media. Suitable media are available from commercial suppliers or may be prepared according to published recipes (e.g. in catalogues of the American Type Culture Collection).

### Expression Hosts

In some embodiments, the host cell is a eukaryotic cell. Suitable eukaryotic host cells include, but are not limited to, fungal cells, algal cells, insect cells, and plant cells. Suitable fungal host cells include, but are not limited to, Ascomycota, Basidiomycota, Deuteromycota, Zygomycota, and Fungi imperfecti. Particularly preferred fungal host cells are yeast cells and filamentous fungal cells. The filamentous fungal host cells of the present invention include all filamentous forms of the subdivision Eumycotina and Oomycota. (Hawksworth et al., In Ainsworth and Bisby's Dictionary of The Fungi, 8^{th} edition, 1995, CAB International, University Press, Cambridge, UK). Filamentous fungi are characterized by a vegetative mycelium with a cell wall composed of chitin, cellulose and other complex polysaccharides. The filamentous fungal host cells of the present invention are morphologically distinct from yeast.

In the present invention a filamentous fungal host cell may be a cell of a species of, but not limited to Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Gliocladium, Humicola, Hypocrea, Myceliophthora, Mucor, Neurospora, Penicillium, Podospora, Phlebia, Piromyces, Pyricularia, Rhizomucor, Rhizopus, Schizophyllum, Scytalidium, Sporotrichum, Talaromyces, Thermoascus, Thielavia, Trametes, Tolypocladium, Trichoderma, Verticillium, Volvariella, or teleomorphs, or anamorphs, and synonyms, basionyms, or taxonomic equivalents thereof.

In some embodiments of the invention, the filamentous fungal host cell is of the Trichoderma species, e.g., T. longibrachiatum, T. viride (e.g., ATCC 32098 and 32086), Hypocrea jecorina or T. reesei (NRRL 15709, ATTC 13631, 56764, 56765, 56466, 56767 and RL-P37 and derivatives thereof; see Sheir-Neiss et al., Appl. Microbiol. Biotechnology, (1984) pp 46-53), T. koningii, and T. harzianum. In addition, the term “Trichoderma” refers to any fungal strain that was previously classified as Trichoderma or currently classified as Trichoderma. In some embodiments of the invention, the filamentous fungal host cell is of the Aspergillus species, e.g., A. awamori, A. funigatus, A. japonicus, A. nidulans, A. niger, A. aculeatus, A. foetidus, A. oryzae, A. sojae, and A. kawachi. (Reference is made to Kelly and Hynes (1985) EMBO J. 4, 475479; NRRL 3112, ATCC 11490, 22342, 44733, and 14331; Yelton M., et al., (1984) Proc. Natl. Acad. Sci. USA, 81, 1470-1474; Tilburn et al., (1982) Gene 26, 205-221; and Johnston, I. L. et al. (1985) EMBO J. 4, 1307-1311). In some embodiments of the invention, the filamentous fungal host cell is of the Chrysosporium species, e.g., C. lucknowense, C. keratinophilum, C. tropicum, C. merdarium, C. Mops, C. pannicola, and C. zonatum. In some embodiments of the invention, the filamentous fungal host cell is of the Myceliophthora species, e.g., M. thermophilia. In some embodiments of the invention, the filamentous fungal host cell is of the Fusarium species, e.g., F. bactridioides, F. cerealis, F. crookwellense, F. culmorum, F. graminearum, F. graminum. F. oxysporum, F. roseum, and F.venenatum. In some embodiments of the invention, the filamentous fungal host cell is of the Neurospora species, e.g., N. crassa. Reference is made to Case, M. E. et al., (1979) Proc. Natl. Acad. Sci. USA, 76, 5259-5263; U.S. Pat. No. 4,486,553; and Kinsey, J. A. and J. A. Rambosek (1984) Molecular and Cellular Biology 4, 117-122. In some embodiments of the invention, the filamentous fungal host cell is of the Humicola species, e.g., H. insolens, H. grisea, and H. lanuginosa. In some embodiments of the invention, the filamentous fungal host cell is of the Mucor species, e.g., M. miehei and M. circinelloides. In some embodiments of the invention, the filamentous fungal host cell is of the Rhizopus species, e.g., R. oryzae and R.niveus. In some embodiments of the invention, the filamentous fungal host cell is of the Penicillum species, e.g., P. purpurogenum, P. chrysogenum, and P. verruculosum. In some embodiments of the invention, the filamentous fungal host cell is of the Thielavia species, e.g., T. terrestris and T. heterothallica. In some embodiments of the invention, the filamentous fungal host cell is of the Tolypocladium species, e.g., T. inflatum and T. geodes. In some embodiments of the invention, the filamentous fungal host cell is of the Trametes species, e.g., T. villosa and T. versicolor. In some embodiments of the invention, the filamentous fungal host cell is of the Sporotrichium species. In some embodiments of the invention, the filamentous fungal host cell is of the Corynascus species.

In the present invention a yeast host cell may be a cell of a species of, but not limited to Candida, Hansenula, Saccharomyces, Schizosaccharomyces, Pichia, Kluyveromyces, and Yarrowia. In some embodiments of the invention, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanolica, Pichia angusta, Kluyveromyces lactis, Candida albicans, or Yarrowia lipolytica.

In some embodiments of the invention, the host cell is an algal cell such as Chlamydomonas (e.g., C. Reinhardtii) and Phormidium (P. sp. ATCC29409).

In other embodiments, the host cell is a prokaryotic cell. Suitable prokaryotic cells include gram positive, gram negative and gram-variable bacterial cells. For example and not for limitation, the host cell may be a species of Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia or Zymomonas. In some embodiments, the host cell is a species of Agrobacterium, Acinetobacter, Azobacter, Bacillus, Bifidobacterium, Buchnera, Geobacillus, Campylobacter, Clostridium, Corynebacterium, Escherichia, Enterococcus, Erwinia, Flavobacterium, Lactobacillus, Lactococcus, Pantoea, Pseudomonas, Staphylococcus, Salmonella, Streptococcus, Streptomyces, or Zymomonas.

In yet other embodiments, the bacterial host strain is non-pathogenic to humans. In some embodiments the bacterial host strain is an industrial strain. Numerous bacterial industrial strains are known and suitable in the present invention.

In some embodiments of the invention, the bacterial host cell is of the Agrobacterium species, e.g., A. radiobacter, A. rhizogenes, and A. rubi. In some embodiments of the invention the bacterial host cell is of the Arthrobacter species, e.g., A. aurescens, A. citreus, A. globformis, A. hydrocarboglutamicus, A. mysorens, A. nicotianae, A. paraffineus, A. protophonniae, A. roseoparqffinus, A. sulfureus, and A. ureafaciens. In some embodiments of the invention the bacterial host cell is of the Bacillus species, e.g., B. thuringensis, B. anthracis, B. megaterium, B. subtilis, B. lentus, B. circulans, B. pumilus, B. lautus, B.coagulans, B. brevis, B. firmus, B. alkaophius, B. licheniformis, B. clausii, B. stearothermophilus, B. halodurans and B. amyloliquefaciens. In particular embodiments, the host cell will be an industrial Bacillus strain including but not limited to B. subtilis, B. pumilus, B. licheniformis, B. megaterium, B. clausii, B. stearothermophilus and B. amyloliquefaciens. Some preferred embodiments of a Bacillus host cell include B. subtilis, B. licheniformis, B. megaterium, B. stearothermophilus and B. amyloliquefaciens. In some embodiments the bacterial host cell is of the Clostridium species, e.g., C. acetobutylicum, C. tetani E88, C. lituseburense, C. saccharobutylicum, C. perfringens, and C. beijerinckii. In some embodiments the bacterial host cell is of the Corynebacterium species e.g., C. glutamicum and C. acetoacidophilum. In some embodiments the bacterial host cell is of the Escherichia species, e.g., E. coli. In some embodiments the bacterial host cell is of the Erwinia species, e.g., E. uredovora, E. carotovora, E. ananas, E. herbicola, E. punctata, and E. terreus. In some embodiments the bacterial host cell is of the Pantoea species, e.g., P. citrea, and P. agglomerans. In some embodiments the bacterial host cell is of the Pseudomonas species, e.g., P. putida, P. aeruginosa, P. mevalonfi, and P. sp. D-01 10. In some embodiments the bacterial host cell is of the Streptococcus species, e.g., S. equisimiles, S. pyogenes, and S. uberis. In some embodiments the bacterial host cell is of the Streptomyces species, e.g., S. ambofaciens, S. achromogenes, S. avermitilis, S. coelicolor, S. aureofaciens, S. aureus, S. fungicidicus, S. griseus, and S. lividans. In some embodiments the bacterial host cell is of the Zymomonas species, e.g., Z. mobilis, and Z. lipolytica.

Strains which may be used in the practice of the invention including both prokaryotic and eukaryotic strains, are readily accessible to the public from a number of culture collections such as American Type Culture Collection (ATCC), Deutsche Sammlung von Mikroorganismen unci Zellkulturen GmbH (DSM), Centraalbureau Voor Schimmel cultures (CBS), and Agricultural Research Service Patent Culture Collection, Northern Regional Research Center (NRRL).

Host cells may be genetically modified to have characteristics that improve protein secretion, protein stability or other properties desirable for expression and/or secretion of a protein. For example, knockout of Alp1 function results in a cell that is protease deficient. Knockout of pyr5 function results in a cell with a pyrimidine deficient phenotype. In particular embodiments host cells are modified to delete endogenous cellulase protein-encoding sequences or otherwise eliminate expression of one or more endogenous cellulases. In one embodiment expression of one or more endogenous cellulases is inhibited to increase production of cellulases of interest. Genetic modification can be achieved by genetic engineering techniques or using classical microbiological techniques, such as chemical or UV mutagenesis and subsequent selection. A combination of recombinant modification and classical selection techniques may be used to produce the organism of interest. Using recombinant technology, nucleic acid molecules can be introduced, deleted, inhibited or modified, in a manner that results in increased yields of cellobiohydrolase within the organism or in the culture. In one genetic engineering approach, homologous recombination can be used to induce targeted gene modifications by specifically targeting a gene in vivo to suppress expression of the encoded protein. In an alternative approach, siRNA, antisense, or ribozyme technology can be used to inhibit gene expression.

In some embodiments, the host cell for expression is a fungal cell (e.g., Myceliophthora thermophila) genetically modified to reduce the amount of endogenous cellobiose dehydrogenase (EC 1.1.3.4) and/or other enzyme (e.g., protease) activity that is secreted by the cell. A variety of methods are known in the art for reducing expression of protein in a cell, including deletion of all or part of the gene encoding the protein and site-specific mutagenesis to disrupt expression or activity of the gene product. See, e.g., Chaveroche et al., 2000, Nucleic Acids Research, 28:22 e97; Cho et al., 2006, MPMI 19: 1, pp. 7-15; Maruyama and Kitamoto, 2008, Biotechnol Lett 30:1811-1817; Takahashi et al., 2004, Mol Gen Genomics 272: 344-352; and You et al., 2009, Arch Micriobiol 191:615-622, the contents of each of which is incorporated by reference herein in its entirety. Random mutagenesis, followed by screening for desired mutations, can also be used. See e.g., Combier et al., 2003, FEMS Microbiol Lett 220:141-8 and Firon et al., 2003, Eukaryot Cell 2:247-55, incorporated by reference herein in its entirety.

In some embodiments, the cell is modified to reduce production of endogenous cellobiose dehydrogenases. In some embodiments, the cell is modified to reduce production of cellobiose dehydrogenase (e.g., CDH1 or CDH2). In some embodiments, the host cell has less than 75%, sometimes less than 50%, sometimes less than 30%, sometimes less than 25%, sometimes less than 20%, sometimes less than 15%, sometimes less than 10%, sometimes less than 5%, and sometimes less than 1% of the cellobiose dehydrogenase (e.g., CDH1 and/or CDH2) activity of the corresponding cell in which the gene is not disrupted.

Exemplary Myceliophthora thermophila cellobiose dehydrogenases are CDH1 (SEQ ID NO:15), encoded by the nucleotide sequence SEQ ID NO:14, and CDH2 (SEQ ID NO:17) encoded by the nucleotide sequence SEQ ID NO:16. The genomic sequence for the Cdh1 encoding CDH1 has accession number AF074951.1. In one approach, gene disruption is achieved using genomic flanking markers (see, e.g., Rothstein, 1983, Methods in Enzymology 101:202-11).

Site-directed mutagenesis may be used to target a particular domain, in some cases, to reduce enzymatic activity (e.g., glucose-methanol-choline oxido-reductase N and C domains of a cellobiose dehydrogenase or heme binding domain of a cellobiose dehydrogenase; see, e.g., Rotsaert et al., 2001, Arch. Biochem. Biophys.390:206-14, which is incorporated by reference herein in its entirety).

### Transformation and Culture

Introduction of a vector or DNA construct into a host cell can be effected by calcium phosphate transfection, DEAE-Dextran mediated transfection, PEG-mediated transformation, electroporation, or other common techniques (See Davis et al., 1986, Basic Methods in Molecular Biology, which is incorporated herein by reference).

The engineered host cells can be cultured in conventional nutrient media modified as appropriate for activating promoters, selecting transformants, or amplifying the cellobiohydrolase polynucleotide. Culture conditions, such as temperature, pH and the like, are those previously used with the host cell selected for expression, and will be apparent to those skilled in the art. As noted, many references are available for the culture and production of many cells, including cells of bacterial, plant, animal (especially mammalian) and archebacterial origin. See e.g., Sambrook, Ausubel, and Berger (all supra), as well as Freshney (1994) Culture of Animal Cells, a Manual of Basic Technique, third edition, Wiley-Liss, New York and the references cited therein; Doyle and Griffiths (1997) Mammalian Cell Culture: Essential Techniques John Wiley and Sons, NY; Humason (1979) Animal Tissue Techniques, fourth edition W.H. Freeman and Company; and Ricciardelli, et al., (1989) In Vitro Cell Dev. Biol. 25:1016-1024, all of which are incorporated herein by reference. For plant cell culture and regeneration, Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); Jones, ed. (1984) Plant Gene Transfer and Expression Protocols, Humana Press, Totowa, N.J. and Plant Molecular Biology (1993) R. R. D. Croy, Ed. Bios Scientific Publishers, Oxford, U.K. ISBN 0 12 198370 6, all of which are incorporated herein by reference. Cell culture media in general are set forth in Atlas and Parks (eds.) The Handbook of Microbiological Media (1993) CRC Press, Boca Raton, Fla., which is incorporated herein by reference. Additional information for cell culture is found in available commercial literature such as the Life Science Research Cell Culture Catalogue (1998) from Sigma-Aldrich, Inc (St Louis, Mo.) (“Sigma-LSRCCC”) and, for example, The Plant Culture Catalogue and supplement (1997) also from Sigma-Aldrich, Inc (St Louis, Mo.) (“Sigma-PCCS”), all of which are incorporated herein by reference.

In some embodiments, cells expressing the cellobiohydrolase polypeptides of the invention are grown under batch or continuous fermentations conditions. Classical batch fermentation is a closed system, wherein the compositions of the medium is set at the beginning of the fermentation and is not subject to artificial alternations during the fermentation. A variation of the batch system is a fed-batch fermentation which also finds use in the present invention. In this variation, the substrate is added in increments as the fermentation progresses. Fed-batch systems are useful when catabolite repression is likely to inhibit the metabolism of the cells and where it is desirable to have limited amounts of substrate in the medium. Batch and fed-batch fermentations are common and well known in the art. Continuous fermentation is an open system where a defined fermentation medium is added continuously to a bioreactor and an equal amount of conditioned medium is removed simultaneously for processing. Continuous fermentation generally maintains the cultures at a constant high density where cells are primarily in log phase growth. Continuous fermentation systems strive to maintain steady state growth conditions. Methods for modulating nutrients and growth factors for continuous fermentation processes as well as techniques for maximizing the rate of product formation are well known in the art of industrial microbiology.

Cell-free transcription/translation systems can also be employed to produce cellobiohydrolase polypeptides using the polynucleotides of the present invention. Several such systems are commercially available. A general guide to in vitro transcription and translation protocols is found in Tymms (1995) In vitro Transcription and Translation Protocols: Methods in Molecular Biology, Volume 37, Garland Publishing, NY, which is incorporated herein by reference.

### IX. Production and Recovery of Cellobiohydrolase Variants

In another aspect, the present invention is directed to a method of making a polypeptide having cellobiohydrolase activity. In some embodiments, the method comprises: providing a host cell transformed with any one of the described cellobiohydrolase polynucleotides of the present invention (e.g., a polynucleotide encoding a polypeptide comprising at least about 70% (or at least about 75%, at least about 80%, at least about 85%, at least about 90%, at least about 95%, at least about 96%, at least about 97%, at least about 98%, or at least about 99%) sequence identity to SEQ ID NO:2 and comprising one or more substitutions or substitution sets as described herein, e.g., in Table 2); culturing the transformed host cell in a culture medium under conditions in which the host cell expresses the encoded cellobiohydrolase polypeptide; and optionally recovering or isolating the expressed cellobiohydrolase polypeptide, or recovering or isolating the culture medium containing the expressed cellobiohydrolase polypeptide. The method further provides optionally lysing the transformed host cells after expressing the encoded cellobiohydrolase polypeptide and optionally recovering or isolating the expressed cellobiohydrolase polypeptide from the cell lysate. The present invention further provides a method of making an cellobiohydrolase polypeptide, said method comprising cultivating a host cell transformed with a cellobiohydrolase polypeptide under conditions suitable for the production of the cellobiohydrolase polypeptide and recovering the cellobiohydrolase polypeptide.

Typically, recovery or isolation of the cellobiohydrolase polypeptide is from the host cell culture medium, the host cell or both, using protein recovery techniques that are well known in the art, including those described herein. Cells are typically harvested by centrifugation, disrupted by physical or chemical means, and the resulting crude extract may be retained for further purification. Microbial cells employed in expression of proteins can be disrupted by any convenient method, including freeze-thaw cycling, sonication, mechanical disruption, or use of cell lysing agents, or other methods, which are well known to those skilled in the art.

The resulting polypeptide may be recovered/isolated and optionally purified by any of a number of methods known in the art. For example, the polypeptide may be isolated from the nutrient medium by conventional procedures including, but not limited to, centrifugation, filtration, extraction, spray-drying, evaporation, chromatography (e.g., ion exchange, affinity, hydrophobic interaction, chromatofocusing, and size exclusion), or precipitation. Protein refolding steps can be used, as desired, in completing the configuration of the mature protein. Finally, high performance liquid chromatography (HPLC) can be employed in the final purification steps. Purification of BGL1 is described in Parry et al., 2001, Biochem. J. 353:117, and Hong et al., 2007, Appl. Microbiol. Biotechnol. 73:1331, both incorporated herein by reference. In addition to the references noted supra, a variety of purification methods are well known in the art, including, for example, those set forth in Sandana (1997) Bioseparation of Proteins, Academic Press, Inc.; Bollag et al. (1996) Protein Methods, 2^{nd} Edition, Wiley-Liss, NY; Walker (1996) The Protein Protocols Handbook Humana Press, NJ; Harris and Angel (1990) Protein Purification Applications: A Practical Approach, IRL Press at Oxford, Oxford, England; Harris and Angel Protein Purification Methods: A Practical Approach, IRL Press at Oxford, Oxford, England; Scopes (1993) Protein Purification: Principles and Practice 3^{rd} Edition, Springer Verlag, NY; Janson and Ryden (1998) Protein Purification: Principles, High Resolution Methods and Applications, Second Edition, Wiley-VCH, NY; and Walker (1998) Protein Protocols on CD-ROM, Humana Press, NJ, all of which are incorporated herein by reference.

Immunological methods may be used to purify cellobiohydrolase polypeptides. In one approach, antibody raised against the cellobiohydrolase polypeptides (e.g., against a polypeptide comprising SEQ ID NO:2 or an immunogenic fragment thereof) using conventional methods is immobilized on beads, mixed with cell culture media under conditions in which the cellobiohydrolase is bound, and precipitated. In a related approach immunochromatography is used.

As noted, in some embodiments the cellobiohydrolase is expressed as a fusion protein including a non-enzyme portion. In some embodiments the cellobiohydrolase sequence is fused to a purification facilitating domain. As used herein, the term “purification facilitating domain” refers to a domain that mediates purification of the polypeptide to which it is fused. Suitable purification domains include metal chelating peptides, histidine-tryptophan modules that allow purification on immobilized metals, a sequence which binds glutathione (e.g., GST), a hemagglutinin (HA) tag (corresponding to an epitope derived from the influenza hemagglutinin protein; Wilson et al., 1984, Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the CBH1a polypeptide is useful to facilitate purification. One expression vector contemplated for use in the compositions and methods described herein provides for expression of a fusion protein comprising a polypeptide of the invention fused to a polyhistidine region separated by an enterokinase cleavage site. The histidine residues facilitate purification on IMIAC (immobilized metal ion affinity chromatography, as described in Porath et al., 1992, Protein Expression and Purification 3:263-281) while the enterokinase cleavage site provides a means for separating the CBH1a polypeptide from the fusion protein. pGEX vectors (Promega; Madison, Wis.) may also be used to express foreign polypeptides as fusion proteins with glutathione S-transferase (GST). In general, such fusion proteins are soluble and can easily be purified from lysed cells by adsorption to ligand-agarose beads (e.g., glutathione-agarose in the case of GST-fusions) followed by elution in the presence of free ligand.

### X. Methods of Using Cellobiohydorlase Variants and Cells Expressing Cellobiohydrolase Variants

The cellobiohydrolase variants as described herein have multiple industrial applications, including but are not limited to, sugar production (e.g. glucose syrups), biofuels production, textile treatment, pulp or paper treatment, and applications in detergents or animal feed. A host cell containing a cellobiohydrolase variant of the present invention may be used without recovery and purification of the recombinant cellobiohydrolase, e.g., for use in a large scale biofermentor. Alternatively, the recombinant cellobiohydrolase variant may be expressed and purified from the host cell. The cellobiohydrolase variants of the present invention may also be used according to the methods of Section III (“Improved Saccharification Process”) of WO 2010/120557, the contents of which are incorporated by reference herein.

The variant cellobiohydrolases that have been described herein are particularly useful for breaking down cellulose to smaller oligosaccharides, disaccharides and monosaccharides. In some embodiments, the variant cellobiohydrolases are useful in saccharification methods. In some embodiments, the variant cellobiohydrolases may be used in combination with other cellulase enzymes including, for example, conventional enzymatic saccharification methods, to produce fermentable sugars.

Therefore, in one aspect the present invention provides a method of producing an end-product from a cellulosic substrate, the method comprising contacting the cellulosic substrate with a cellobiohydrolase variant as described herein (and optionally other cellulases) under conditions in which fermentable sugars are produced, and contacting fermentable sugars with a microorganism in a fermentation to produce the end-product. In some embodiments, the method further comprises pretreating the cellulosic substrate to increase its susceptibility to hydrolysis prior to contacting the cellulosic substrate with the cellobiohydrolase variant (and optionally other cellulases).

In some embodiments, enzyme compositions comprising the cellobiohydrolase variants of the present invention may be reacted with a biomass substrate in the range of about 25° C. to 100° C., about 30° C. to 90° C., about 30° C. to 80° C., and about 30° C. to 70° C. Also the biomass may be reacted with the cellobiohydrolase enzyme compositions at about 25° C., at about 30° C., at about 35° C., at about 40° C., at about 45° C., at about 50° C., at about 55° C., at about 60° C., at about 65° C., at about 70° C., at about 75° C., at about 80° C., at about 85° C., at about 90° C., at about 95° C. and at about 100° C. Generally the pH range will be from about pH 3.0 to 8.5, pH 3.5 to 8.5, pH 4.0 to 7.5, pH 4.0 to 7.0 and pH 4.0 to 6.5. The incubation time may vary for example from 1.0 to 240 hours, from 5.0 to 180 hrs and from 10.0 to 150 hrs. For example, the incubation time will be at least 1 hr, at least 5 hrs, at least 10 hrs, at least 15 hrs, at least 25 hrs, at least 50 hr, at least 100 hrs, at least 180 hrs, and the like. Incubation of the cellulase under these conditions and subsequent contact with the substrate may result in the release of substantial amounts of fermentable sugars (e.g., glucose) from the substrate (e.g., glucose when β-glucosidase is also included in the incubation mixture). For example at least 20%, at least 30%, at least 40%, at least 50%, at least 60%, at least 70%, at least 80%, at least 90% or more fermentable sugar (e.g., glucose) may be available as compared to the release of sugar by a wild-type polypeptide.

In some embodiments, an end-product of a fermentation is any product produced by a process including a fermentation step using a fermenting organism. Examples of end-products of a fermentation include, but are not limited to, alcohols (e.g., fuel alcohols such as ethanol and butanol), organic acids (e.g., citric acid, acetic acid, lactic acid, gluconic acid, and succinic acid), glycerol, ketones, diols, amino acids (e.g., glutamic acid), antibiotics (e.g., penicillin and tetracycline), vitamins (e.g., beta-carotine and B12), hormones, and fuel molecules other than alcohols (e.g., hydrocarbons).

In some embodiments, the fermentable sugars produced by the methods of the present invention may be used to produce an alcohol (such as, for example, ethanol, butanol, and the like). The variant cellobiohydrolases of the present invention may be utilized in any method used to generate alcohols or other biofuels from cellulose, and are not limited necessarily to those described herein. Two methods commonly employed are the separate saccharification and fermentation (SHF) method (see, Wilke et al., Biotechnol. Bioengin. 6:155-75 (1976)) or the simultaneous saccharification and fermentation (SSF) method disclosed for example in U.S. Pat. Nos. 3,990,944 and 3,990,945.

The SHF method of saccharification comprises the steps of contacting a cellulase with a cellulose containing substrate to enzymatically break down cellulose into fermentable sugars (e.g., monosaccharides such as glucose), contacting the fermentable sugars with an alcohol-producing microorganism to produce alcohol (e.g., ethanol or butanol) and recovering the alcohol. In some embodiments, the method of consolidated bioprocessing (CBP) can be used, where the cellulase production from the host is simultaneous with saccharification and fermentation either from one host or from a mixed cultivation.

In addition to SHF methods, a SSF method may be used. In some cases, SSF methods result in a higher efficiency of alcohol production than is afforded by the SHF method (Drissen et al., Biocatalysis and Biotransformation 27:27-35 (2009). One disadvantage of SSF over SHF is that higher temperatures are required for SSF than for SHF. In one embodiment, the present invention claims cellobiohydrolase polypeptides that have higher thermostability than a wild-type cellobiohydrolase and one practicing the present invention could expect an increase in ethanol production if using the cellulases described here in combination with SSF.

For cellulosic substances to be used effectively as substrates for the saccharification reaction in the presence of a cellulase of the present invention, it is desirable to pretreat the substrate. Means of pretreating a cellulosic substrate are known in the art, including but not limited to chemical pretreatment (e.g., ammonia pretreatment, dilute acid pretreatment, dilute alkali pretreatment, or solvent exposure), physical pretreatment (e.g., steam explosion or irradiation), mechanical pretreatment (e.g., grinding or milling) and biological pretreatment (e.g., application of lignin-solubilizing microorganisms), and the present invention is not limited by such methods.

Any alcohol producing microorganism such as those known in the art, e.g., Saccharomyces cerevisiae, can be employed with the present invention for the fermentation of fermentable sugars to alcohols and other end-products.

The fermentable sugars produced from the use of one or more cellobiohydrolase variants encompassed by the invention may be used to produce other end-products besides alcohols, such as but not limited to other biofuels compounds, acetone, an amino acid (e.g., glycine, lysine, and the like), organic acids (e.g., lactic acids and the like), glycerol, ascorbic acid, a diol (e.g., 1,3-propanediol, butanediol, and the like), vitamins, hormones, antibiotics, other chemicals, and animal feeds.

The cellobiohydrolase variants as described herein are further useful in the pulp and paper industry. In the pulp and paper industry, neutral cellulases can be used, for example, in deinking of different recycled papers and paperboards having neutral or alkaline pH, in improving the fiber quality, or increasing the drainage in paper manufacture. Other examples include, for example, the removal of printing paste thickener and excess dye after textile printing.

### Lignocellulosic Feedstocks

The term “lignocellulosic feedstock” refers to any type of plant biomass comprising lignin and cellulose, such as, but not limited to, non-woody plant biomass, cultivated crops such as, but not limited to grasses, for example, but not limited to, C4 grasses, such as switch grass, cord grass, rye grass, miscanthus, reed canary grass, or a combination thereof, sugar processing residues, for example, but not limited to, baggase, such as sugar cane bagasse, beet pulp, or a combination thereof, agricultural residues, for example, but not limited to, soybean stover, corn stover, rice straw, sugar cane straw, rice hulls, barley straw, corn cobs, wheat straw, canola straw, oat straw, oat hulls, corn fiber, or a combination thereof, forestry biomass for example, but not limited to, recycled wood pulp fiber, sawdust, hardwood, for example aspen wood, softwood, or a combination thereof. Furthermore, the lignocellulosic feedstock may comprise cellulosic waste material or forestry waste materials such as, but not limited to, newsprint, cardboard and the like. Lignocellulosic feedstocks may comprise one species of fiber or, alternatively, lignocellulosic feedstock may comprise a mixture of fibers that originate from different lignocellulosic feedstocks. In addition, the lignocellulosic feedstock may comprise fresh lignocellulosic feedstock, partially dried lignocellulosic feedstock, fully dried lignocellulosic feedstock, or a combination thereof.

In some embodiments, lignocellulosic feedstocks comprise cellulose in an amount greater than about 20%, more preferably greater than about 30%, more preferably greater than about 40% (w/w). For example, the lignocellulosic material may comprise from about 20% to about 90% (w/w) cellulose, or any amount therebetween. Furthermore, in some embodiments, the lignocellulosic feedstock comprises lignin in an amount greater than about 10%, more typically in an amount greater than about 15% (w/w). The lignocellulosic feedstock may also comprise small amounts of sucrose, fructose and starch. The lignocellulosic feedstock is generally first subjected to size reduction by methods including, but not limited to, milling, grinding, agitation, shredding, compression/expansion, or other types of mechanical action. Size reduction by mechanical action can be performed by any type of equipment adapted for the purpose, for example, but not limited to, hammer mills, tub-grinders, roll presses, refiners and hydrapulpers. At least 90% by weight of the particles produced from the size reduction may have a length less than between about 1/16 and about 4 in. The measurement may be a volume or a weight average length.

The preferable equipment for the particle size reduction is a hammer mill or shredder. Subsequent to size reduction, the feedstock is typically slurried in water. This allows the feedstock to be pumped.

Lignocellulosic feedstocks of particle size less than about 6 inches may not require size reduction.

The feedstock may be slurried prior to pretreatment. In one embodiment of the invention, the consistency of the feedstock slurry is between about 2% and about 30% and more typically between about 4% and about 15%. Optionally, the slurry is subjected to a water or acid soaking operation prior to pretreatment.

Prior to pretreatment, the slurry may be dewatered using known methodologies to reduce steam and chemical usage. Examples of dewatering devices include pressurized screw presses, such as those described in WO 2010/022511 (incorporated herein by reference), pressurized filters and extruders.

A pretreated lignocellulosic feedstock, or pretreated lignocellulose, is a lignocellulosic feedstock that has been subjected to physical and/or chemical processes to make the fiber more accessible and/or receptive to the actions of cellulolytic enzymes.

The pretreatment may be carried out to hydrolyze the hemicellulose, or a portion thereof, that is present in the lignocellulosic feedstock to monomeric pentose and hexose sugars, for example xylose, arabinose, mannose, galactose, or a combination thereof. For example, the pretreatment may be carried out so that nearly complete hydrolysis of the hemicellulose and a small amount of conversion of cellulose to glucose occurs. During the pretreatment, typically an acid concentration in the aqueous slurry from about 0.02% (w/w) to about 2% (w/w), or any amount therebetween, is used for the treatment of the lignocellulosic feedstock. The acid may be, but is not limited to, hydrochloric acid, nitric acid, or sulfuric acid. For example, the acid used during pretreatment is sulfuric acid.

One method of performing acid pretreatment of the feedstock is steam explosion using the process conditions set out in U.S. Pat. No. 4,461,648. Another method of pretreating the feedstock slurry involves continuous pretreatment, meaning that the lignocellulosic feedstock is pumped though a reactor continuously. Continuous acid pretreatment is familiar to those skilled in the art; see, for example, U.S. Pat. No. 7,754,457.

The pretreatment may also be conducted with alkali. In contrast to acid pretreatment, pretreatment with alkali may not hydrolyze the hemicellulose component of the feedstock. For example, the alkali may react with acidic groups present on the hemicellulose to open up the surface of the substrate. The addition of alkali may also alter the crystal structure of the cellulose so that it is more amenable to hydrolysis. Examples of alkali that may be used in the pretreatment include ammonia, ammonium hydroxide, potassium hydroxide, and sodium hydroxide.

An example of a suitable alkali pretreatment is Ammonia Freeze Explosion, Ammonia Fiber Explosion or Ammonia Fiber Expansion (“AFEX” process). According to this process, the lignocellulosic feedstock is contacted with ammonia or ammonium hydroxide in a pressure vessel for a sufficient time to enable the ammonia or ammonium hydroxide to alter the crystal structure of the cellulose fibers. The pressure is then rapidly reduced, which allows the ammonia to flash or boil and explode the cellulose fiber structure. (See U.S. Pat. Nos. 5,171,592; 5,037,663; 4,600,590; 6,106,888; 4,356,196; 5,939,544; 6,176,176; 5,037,663 and 5,171,592). The flashed ammonia may then be recovered according to known processes.

Dilute ammonia pretreatment utilizes more dilute solutions of ammonia or ammonium hydroxide than AFEX (see WO2009/045651 and US 2007/0031953). Such a pretreatment process may or may not produce any monosaccharides.

Yet a further non-limiting example of a pretreatment process for use in the present invention includes chemical treatment of the feedstock with organic solvents. Organic liquids in pretreatment systems are described by Converse et al. (U.S. Pat. No. 4,556,430; incorporated herein by reference), and such methods have the advantage that the low boiling point liquids easily can be recovered and reused. Other pretreatments, such as the Organosolv™ process, also use organic liquids (see U.S. Pat. No. 7,465,791, which is also incorporated herein by reference). Subjecting the feedstock to pressurized water may also be a suitable pretreatment method (see Weil et al. (1997) Appl. Biochem. Biotechnol. 68(1-2): 21-40, which is incorporated herein by reference).

The pretreated lignocellulosic feedstock may be processed after pretreatment by any of several steps, such as dilution with water, washing with water, buffering, filtration, or centrifugation, or a combination of these processes, prior to enzymatic hydrolysis, as is familiar to those skilled in the art.

The pretreatment produces a pretreated feedstock composition (e.g., a pretreated feedstock slurry) that contains a soluble component including the sugars resulting from hydrolysis of the hemicellulose, optionally acetic acid and other inhibitors, and solids including unhydrolyzed feedstock and lignin.

The soluble components of the pretreated feedstock composition may be separated from the solids to produce a soluble fraction. The soluble fraction, which includes the sugars released during pretreatment and other soluble components, including inhibitors, may then be sent to fermentation. It will be understood, however, that if the hemicellulose is not effectively hydrolyzed during the pretreatment, it may be desirable to include a further hydrolysis step or steps with enzymes or by further alkali or acid treatment to produce fermentable sugars. The foregoing separation may be carried out by washing the pretreated feedstock composition with an aqueous solution to produce a wash stream, and a solids stream comprising the unhydrolyzed, pretreated feedstock. Alternatively, the soluble component is separated from the solids by subjecting the pretreated feedstock composition to a solids-liquid separation, using known methods such as centrifugation, microfiltration, plate and frame filtration, cross-flow filtration, pressure filtration, vacuum filtration, and the like. Optionally, a washing step may be incorporated into the solids-liquids separation. The separated solids, which contain cellulose, may then be sent to enzymatic hydrolysis with cellulase enzymes in order to convert the cellulose to glucose.

The pretreated feedstock composition may be fed to the fermentation without separation of the solids contained therein. After the fermentation, the unhydrolyzed solids may be subjected to enzymatic hydrolysis with cellulase enzymes to convert the cellulose to glucose.

Prior to hydrolysis with cellulase enzymes, the pH of the pretreated feedstock slurry may be adjusted to a value that is amenable to the cellulase enzymes, which is typically between about 4 and about 6, although the pH can be higher if alkalophilic cellulases are used.

### Enzyme Mixtures

In another aspect, the invention provides an enzyme mixture that comprises a cellobiohydrolase variant polypeptide as described herein. In some embodiments, the enzymes of the enzyme mixture may be secreted from a host cell and in other embodiments, the enzymes of the enzyme mixture may not be secreted. The enzyme mixture may be cell-free, or in alternative embodiments, may not be separated from host cells that secrete an enzyme mixture component. A cell-free enzyme mixture typically comprises enzymes that have been separated from any cells. Cell-free enzyme mixtures can be prepared by any of a variety of methodologies that are known in the art, such as filtration or centrifugation methodologies. In certain embodiments, the enzyme mixture can be, for example, partially cell-free, substantially cell-free, or entirely cell-free. In some embodiments, one or more enzymes of the enzyme mixture are not secreted by the host cell. The cells may be lysed to release the enzyme(s). Enzymes may be recovered from the cell lysate or the cell lysate may be combined, with partial purification or without further purification, with the substrate.

The cellobiohydrolase variant and any additional enzymes present in an enzyme mixture may be secreted from a single genetically modified host cell or by different microbes in combined or separate fermentations. Similarly, the cellobiohydrolase variant and any additional enzymes present in the enzyme mixture may be expressed individually or in sub-groups from different strains of different organisms and the enzymes combined in vitro to make the enzyme mixture. It is also contemplated that the cellobiohydrolase variant and any additional enzymes in the enzyme mixture may be expressed individually or in sub-groups from different strains of a single organism, and the enzymes combined to make the enzyme mixture. In some embodiments, all of the enzymes are expressed from a single host organism, such as a genetically modified fungal cell.

In some embodiments, the enzyme mixture comprises other types of cellulases, selected from but not limited to cellobiohydrolase, endoglucanase, β-glucosidase, and glycoside hydrolase 61 protein (GH61) cellulases. These enzymes may be wild-type or recombinant enzymes. In some embodiments, the cellobiohydrolase is a type 2 cellobiohydrolase, e.g., a T. reesei cellobiohydrolase II. In some embodiments, the endoglucanase comprises a catalytic domain derived from the catalytic domain of a Streptomyces avermitilis endoglucanase. See US 2010/0267089, incorporated herein by reference. In some embodiments, the at least one cellulase is derived from Acidothermus cellulolyticus, Thermobifida fusca, Humicola grisea, Myceliophthora thermophilia, Chaetomium thermophilum, Acremonium sp., Thielavia sp, Trichoderma reesei, Aspergillus sp., or a Chrysosporium sp. Cellulase enzymes of the cellulase mixture work together resulting in decrystallization and hydrolysis of the cellulose from a biomass substrate to yield fermentable sugars, such as but not limited to glucose (See Brigham et al., 1995, in Handbook on Bioethanol (C. Wyman ed.) pp 119-141, Taylor and Francis, Washington D.C., which is incorporated herein by reference).

Cellulase mixtures for efficient enzymatic hydrolysis of cellulose are known (see, e.g., Viikari et al., 2007, “Thermostable enzymes in lignocellulose hydrolysis” Adv Biochem Eng Biotechnol 108:121-45, and US Pat. publications US 2009/0061484; US 2008/0057541; and US 2009/0209009 to logen Energy Corp.), each of which is incorporated herein by reference for all purposes. In some embodiments, mixtures of purified naturally occurring or recombinant enzymes are combined with cellulosic feedstock or a product of cellulose hydrolysis. Alternatively or in addition, one or more cell populations, each producing one or more naturally occurring or recombinant cellulases, may be combined with cellulosic feedstock or a product of cellulose hydrolysis.

In some embodiments, the enzyme mixture comprises an isolated CBH1a variant as described herein and at least one or more of an isolated type 2 cellobiohydrolase such as a CBH2b, an isolated endoglucanase (EG) such as a type 2 endoglucanase (EG2) and/or a type 1 endoglucanase (EG1), an isolated (3-glucosidase (BgI), and an isolated glycoside hydrolase 61 protein (GH61). In some embodiments, at least 20%, at least 25%, at least 30%, at least 35%, at least 40%, at least 45%, or at least 50% of the enzyme mixture is a CBH1a variant. In some embodiments, the enzyme mixture further comprises a type 2 cellobiohydrolase (e.g., CBH2b), and the CBH1a variant and the CBH2b together comprise at least 25%, at least 30%, at least 35%, at least 40%, at least 45%, at least 50%, at least 55%, at least 60%, at least 65%, at least 70%, at least 75%, or at least 80% of the enzyme mixture. In some embodiments, the enzyme mixture further comprises a 3-glucosidase (BgI), and the CBH1a variant, the CBH2b, and the BgI together comprise at least 30%, at least 35%, at least 40%, at least 45%, at least 50%, at least 55%, at least 60%, at least 65%, at least 70%, at least 75%, at least 80%, or at least 85% of the enzyme mixture. In some embodiments, the enzyme mixture further comprises an endoglucanase (EG), and the CBH1a variant, the CBH2b, the BgI, and the EG together comprise at least 35%, at least 40%, at least 45%, at least 50%, at least 55%, at least 60%, at least 65%, at least 70%, at least 75%, at least 80%, at least 85%, or at least 90% of the enzyme mixture. In some embodiments, the enzyme mixture comprises a CBH1a variant as described herein, a type 2b cellobiohydrolase (CBH2b), a β-glucosidase (BgI), an endoglucanase (EG), and a glycoside hydrolase 61 protein (GH61). In some embodiments, the enzyme mixture composition comprises isolated cellulases in the following proportions by weight (wherein the total weight of the cellulases is 100%): about 20%-10% of EG, about 20%-10% of BgI, about 20%-25% of CBH2b, about 10%-30% of GH61, and about 30%-25% of a CBH1a variant of the present invention. In some embodiments, the enzyme mixture composition comprises isolated cellulases in the following proportions by weight: about 20%-10% of EG, about 25%-15% of BgI, about 25%-30% of CBH2b, about 10%-15% of GH61, and about 20%-30% of a CBH1a variant of the present invention. In some embodiments, the enzyme mixture composition comprises isolated cellulases in the following proportions by weight: about 10%-15% of EG, about 20%-25% of BgI, 25%-35% of CBH2b, about 15%-5% of Gh61, and about 30%-20% of a CBH1a variant of the present invention. In some embodiments, the enzyme mixture composition comprises isolated cellulases in the following proportions by weight: about 15%-5% of EG, about 15%-10% of BgI, 40%-10% of CBH2b, about 25%-5% of GH61, and about 45%-30% of a CBH1a variant of the present invention. in some embodiments, the enzyme mixture composition comprises isolated cellulases in the following proportions by weight: about 10% of EG, about 15% of BgI, about 10% of a CBH2b, about 25% of GH61, and about 40% of a CBH1a variant of the present invention. In some embodiments, the enzyme mixture composition comprises isolated cellulases in the following proportions by weight: about 0% of EG, about 15%-10% of BgI, 30%-40% of a CBH2b, about 15%-10% of GH61, and about 30%-40% of a CBH1a variant of the present invention. In some embodiments, the enzyme component comprises more than 1 CBH1a variant (e.g., 2, 3 or 4 different CBH1a variants as disclosed herein). In some embodiments, an enzyme mixture composition of the invention can also contain one or more additional proteins such as those listed below. In some embodiments, an enzyme mixture composition of the invention can also contain one or more additional enzymes other than the EG, BgI, CBH2b, GH61, and/or CBH1a variant recited herein, such as the enzymes listed below. In some embodiments, an enzyme mixture composition of the invention can also contain one or more additional cellulases other than the EG, BgI, CBH2b, GH61, and/or CBH1a variant recited herein.

A cellobiohydrolase variant polypeptide of the invention may also be present in mixtures with non-cellulase enzymes that degrade cellulose, hemicellulose, pectin, and/or lignocellulose.

A “hemicellulase” as used herein, refers to a polypeptide that can catalyze hydrolysis of hemicellulose into small polysaccharides such as oligosaccharides, or monomeric saccharides. Hemicellullases include xylan, glucuonoxylan, arabinoxylan, glucomannan and xyloglucan. Hemicellulases include, for example, the following: endoxylanases, β-xylosidases, α-L-arabinofuranosidases, α-D-glucuronidases, feruloyl esterases, coumarolyl esterases, α-galactosidases, β-galactosidases, β-mannanases, and β-mannosidases. An enzyme mixture may therefore comprise a cellobiohydrolase variant of the invention and one or more hemicellulases.

An endoxylanase (EC 3.2.1.8) catalyzes the endohydrolysis of 1,4-β-D-xylosidic linkages in xylans. This enzyme may also be referred to as endo-1,4-β-xylanase or 1,4-β-D-xylan xylanohydroiase. An alternative is EC 3.2.1.136, a glucuronoarabinoxylan endoxylanase, an enzyme that is able to hydrolyse 1,4 xylosidic linkages in glucuronoarabinoxylans.

A β-xylosidase (EC 3.2.1.37) catalyzes the hydrolysis of 1,4-β-D-xylans, to remove successive D-xylose residues from the non-reducing termini. This enzyme may also be referred to as xylan 1,4-β-xylosidase, 1,4-β-D-xylan xylohydrolase, exo-1,4-β-xylosidase, or xylobiase.

An α-L-arabinofuranosidase (EC 3.2.1.55) catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, arabinofuranosidase, polysaccharide alpha-L-arabinofuranosidase, alpha-L-arabinofuranoside hydrolase, L-arabinosidase and alpha-L-arabinanase.

An alpha-glucuronidase (EC 3.2.1.139) catalyzes the hydrolysis of an alpha-D-glucuronoside to D-glucuronate and an alcohol.

An acetylxylanesterase (EC 3.1.1.72) catalyzes the hydrolysis of acetyl groups from polymeric xylan, acetylated xylose, acetylated glucose, alpha-napthyl acetate, and p-nitrophenyl acetate.

A feruloyl esterase (EC 3.1.1.73) has 4-hydroxy-3-methoxycinnamoyl-sugar hydrolase activity (EC 3.1.1.73) that catalyzes the hydrolysis of the 4-hydroxy-3-methoxycinnamoyl (feruloyl) group from an esterified sugar, which is usually arabinose in “natural” substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II.

A coumaroyl esterase (EC 3.1.1.73) catalyzes a reaction of the form: coumaroyl-saccharide+H(2)0=coumarate+saccharide. The saccharide may be, for example, an oligosaccharide or a polysaccharide. This enzyme may also be referred to as trans-4-coumaroyl esterase, trans-p-coumaroyl esterase, p-coumaroyl esterase or p-coumaric acid esterase. The enzyme also falls within EC 3.1.1.73 so may also be referred to as a feruloyl esterase.

An a-galactosidase (EC 3.2.1.22) catalyzes the hydrolysis of terminal, non-reducing α-D-galactose residues in α-D-galactosides, including galactose oligosaccharides, galactomannans, galactans and arabinogalactans. This enzyme may also be referred to as melibiase.

A β-galactosidase (EC 3.2.1.23) catalyzes the hydrolysis of terminal non-reducing β-D-galactose residues in β-D-galactosides. Such a polypeptide may also be capable of hydrolyzing α-L-arabinosides. This enzyme may also be referred to as exo-(1->4)-β-D-galactanase or lactase.

A β-mannanase (EC 3.2.1.78) catalyzes the random hydrolysis of 1,4-β-D-mannosidic linkages in mannans, galactomannans and glucomannans. This enzyme may also be referred to as mannan endo-1,4-β-mannosidase or endo-1,4-mannanase.

A β-mannosidase (EC 3.2.1.25) catalyzes the hydrolysis of terminal, non-reducing β-D-mannose residues in β-D-mannosides. This enzyme may also be referred to as mannanase or mannase.

A glucoamylase (EC 3.2.1.3) is an enzyme which catalyzes the release of D-glucose from non-reducing ends of oligo- and poly-saccharide molecules. Glucoamylase is also generally considered a type of amylase known as amylo-glucosidase.

An amylase (EC 3.2.1.1) is a starch cleaving enzyme that degrades starch and related compounds by hydrolyzing the α-1,4 and/or α-1,6 glucosidic linkages in an endo- or an exo-acting fashion. Amylases include α-amylases (EC 3.2.1.1); β-amylases (3.2.1.2), amylo-amylases (EC 3.2.1.3), α-glucosidases (EC 3.2.1.20), pullulanases (EC 3.2.1.41), and isoamylases (EC 3.2.1.68). In some embodiments, the amylase is an α-amylase.

One or more enzymes that degrade pectin may also be included in an enzyme mixture that comprises a cellobiohydrolase variant of the invention. A pectinase catalyzes the hydrolysis of pectin into smaller units such as oligosaccharide or monomeric saccharides. An enzyme mixture may comprise any pectinase, for example an endo-polygalacturonase, a pectin methyl esterase, an endo-galactanase, a pectin acetyl esterase, an endo-pectin lyase, pectate lyase, alpha rhamnosidase, an exo-galacturonase, an exo-polygalacturonate lyase, a rhamnogalacturonan hydrolase, a rhamnogalacturonan lyase, a rhamnogalacturonan acetyl esterase, a rhamnogalacturonan galacturonohydrolase or a xylogalacturonase.

An endo-polygalacturonase (EC 3.2.1.15) catalyzes the random hydrolysis of 1,4-a-D-galactosiduronic linkages in pectate and other galacturonans. This enzyme may also be referred to as polygalacturonase pectin depolymerase, pectinase, endopolygalacturonase, pectolase, pectin hydrolase, pectin polygalacturonase, poly-α-1,4-galacturonide glycanohydrolase, endogalacturonase; endo-D-galacturonase or poly(1,4-α-D-galacturonide) glycanohydrolase.

A pectin methyl esterase (EC 3.1.1.11) catalyzes the reaction: pectin+n H₂O=n methanol+pectate. The enzyme may also been known as pectinesterase, pectin demethoxylase, pectin methoxylase, pectin methylesterase, pectase, pectinoesterase or pectin pectylhydrolase.

An endo-galactanase (EC 3.2.1.89) catalyzes the endohydrolysis of 1,4-β-D-galactosidic linkages in arabinogalactans. The enzyme may also be known as arabinogalactan endo-1,4-β-galactosidase, endo-1,4-β-galactanase, galactanase, arabinogalactanase or arabinogalactan 4-β-D-galactanohydrolase.

A pectin acetyl esterase catalyzes the deacetylation of the acetyl groups at the hydroxyl groups of GalUA residues of pectin.

An endo-pectin lyase (EC 4.2.2.10) catalyzes the eliminative cleavage of (1→4)-α-D-galacturonan methyl ester to give oligosaccharides with 4-deoxy-6-O-methyl-α-D-galact-4-enuronosyl groups at their non-reducing ends. The enzyme may also be known as pectin lyase, pectin trans-eliminase; endo-pectin lyase, polymethylgalacturonic transeliminase, pectin methyltranseliminase, pectolyase, PL, PNL or PMGL or (1→4)-6-O-methyl-α-D-galacturonan lyase.

A pectate lyase (EC 4.2.2.2) catalyzes the eliminative cleavage of (1→4)-α-D-galacturonan to give oligosaccharides with 4-deoxy-α-D-galact-4-enuronosyl groups at their non-reducing ends. The enzyme may also be known polygalacturonic transeliminase, pectic acid transeliminase, polygalacturonate lyase, endopectin methyltranseliminase, pectate transeliminase, endogalacturonate transeliminase, pectic acid lyase, pectic lyase, α-1,4-D-endopolygalacturonic acid lyase, PGA lyase, PPase-N, endo-α-1,4-polygalacturonic acid lyase, polygalacturonic acid lyase, pectin trans-eliminase, polygalacturonic acid trans-eliminase or (1→4)-α-D-galacturonan lyase.

An alpha rhamnosidase (EC 3.2.1.40) catalyzes the hydrolysis of terminal non-reducing α-L-rhamnose residues in α-L-rhamnosides or alternatively in rhamnogalacturonan. This enzyme may also be known as α-L-rhamnosidase T, α-L-rhamnosidase N or α-L-rhamnoside rhamnohydrolase.

An exo-galacturonase (EC 3.2.1.82) hydrolyzes pectic acid from the non-reducing end, releasing digalacturonate. The enzyme may also be known as exo-poly-a-galacturonosidase, exopolygalacturonosidase or exopolygalacturanosidase.

An exo-galacturonase (EC 3.2.1.67) catalyzes a reaction of the following type: (1,4-α-D-galacturonide)n+H2O=(1,4-α-D-galacturonide)n-i+D-galacturonate. The enzyme may also be known as galacturan 1,4-α-galacturonidase, exopolygalacturonase, poly(galacturonate) hydrolase, exo-D-galacturonase, exo-D-galacturonanase, exopoly-D-galacturonase or poly(1,4-α-D-galacturonide) galacturonohydrolase.

An exopolygalacturonate lyase (EC 4.2.2.9) catalyzes eliminative cleavage of 4-(4-deoxy-α-D-galact-4-enuronosyl)-D-galacturonate from the reducing end of pectate, i.e. de-esterified pectin. This enzyme may be known as pectate disaccharide-lyase, pectate exo-lyase, exopectic acid transeliminase, exopectate lyase, exopolygalacturonic acid-trans-eliminase, PATE, exo-PATE, exo-PGL or (1→4)-α-D-galacturonan reducing-end-disaccharide-lyase.

A rhamnogalacturonan hydrolyzes the linkage between galactosyluronic acid acid and rhamnopyranosyl in an endo-fashion in strictly alternating rhamnogalacturonan structures, consisting of the disaccharide [(1,2-alpha-L-rhamnoyl-(1,4)-alpha-galactosyluronic acid].

A rhamnogalacturonan lyase cleaves α-L-Rhap-(1-4)-α-D-GalpA linkages in an endo-fashion in rhamnogalacturonan by beta-elimination.

A rhamnogalacturonan acetyl esterase catalyzes the deacetylation of the backbone of alternating rhamnose and galacturonic acid residues in rhamnogalacturonan.

A rhamnogalacturonan galacturonohydrolase hydrolyzes galacturonic acid from the non-reducing end of strictly alternating rhamnogalacturonan structures in an exo-fashion. This enzyme may also be known as xylogalacturonan hydrolase.

An endo-arabinanase (EC 3.2.1.99) catalyzes endohydrolysis of 1,5-a-arabinofuranosidic linkages in 1,5-arabinans. The enzyme may also be know as endo-arabinase, arabinan endo-1,5-α-L-arabinosidase, endo-1,5-α-L-arabinanase, endo-α-1,5-arabanase; endo-arabanase or 1,5-α-L-arabinan 1,5-α-L-arabinanohydrolase.

One or more enzymes that participate in lignin degradation may also be included in an enzyme mixture that comprises a cellobiohydrolase variant of the invention. Enzymatic lignin depolymerization can be accomplished by lignin peroxidases, manganese peroxidases, laccases and cellobiose dehydrogenases (CDH), often working in synergy. These extracellular enzymes are often referred to as lignin-modifying enzymes or LMEs. Three of these enzymes comprise two glycosylated heme-containing peroxidases: lignin peroxidase (LIP); Mn-dependent peroxidase (MNP); and, a copper-containing phenoloxidase laccase (LCC).

Laccase: Laccases are copper containing oxidase enzymes that are found in many plants, fungi and microorganisms. Laccases are enzymatically active on phenols and similar molecules and perform a one electron oxidation. Laccases can be polymeric and the enzymatically active form can be a dimer or trimer.

Mn-dependent peroxidase: The enzymatic activity of Mn-dependent peroxidase (MnP) in is dependent on Mn2+. Without being bound by theory, it has been suggested that the main role of this enzyme is to oxidize Mn2+ to Mn3+ (Glenn et al. (1986) Arch. Biochem. Biophys. 251:688-696). Subsequently, phenolic substrates are oxidized by the Mn3+ generated.

Lignin peroxidase: Lignin peroxidase is an extracellular heme that catalyses the oxidative depolymerization of dilute solutions of polymeric lignin in vitro. Some of the substrates of LiP, most notably 3,4-dimethoxybenzyl alcohol (veratryl alcohol, VA), are active redox compounds that have been shown to act as redox mediators. VA is a secondary metabolite produced at the same time as LiP by ligninolytic cultures of P. chrysosporium and without being bound by a theory, has been proposed to function as a physiological redox mediator in the LiP-catalysed oxidation of lignin in vivo (Harvey, et al. (1986) FEBS Lett. 195, 242-246).

An enzymatic mixture comprising a cellobiohydrolase variant of the invention may further comprise at least one of the following: a protease or a lipase that participates in cellulose degradation.

“Protease” includes enzymes that hydrolyze peptide bonds (peptidases), as well as enzymes that hydrolyze bonds between peptides and other moieties, such as sugars (glycopeptidases). Many proteases are characterized under EC 3.4, and are suitable for use in the invention. Some specific types of proteases include, cysteine proteases including pepsin, papain and serine proteases including chymotrypsins, carboxypeptidases and metalloendopeptidases.

“Lipase” includes enzymes that hydrolyze lipids, fatty acids, and acylglycerides, including phosphoglycerides, lipoproteins, diacylglycerols, and the like. In plants, lipids are used as structural components to limit water loss and pathogen infection. These lipids include waxes derived from fatty acids, as well as cutin and suberin.

An enzyme mixture that comprises a cellobiohydrolase variant of the invention may also comprise at least one expansin or expansin-like protein, such as a swollenin (see Salheimo et al., Eur. J. Biohem. 269, 4202-4211, 2002) or a swollenin-like protein.

Expansins are implicated in loosening of the cell wall structure during plant cell growth. Expansins have been proposed to disrupt hydrogen bonding between cellulose and other cell wall polysaccharides without having hydrolytic activity. In this way, they are thought to allow the sliding of cellulose fibers and enlargement of the cell wall. Swollenin, an expansin-like protein contains an N-terminal Carbohydrate Binding Module Family 1 domain (CBD) and a C-terminal expansin-like domain. For the purposes of this invention, an expansin-like protein or swollenin-like protein may comprise one or both of such domains and/or may disrupt the structure of cell walls (such as disrupting cellulose structure), optionally without producing detectable amounts of reducing sugars.

An enzyme mixture that comprises a cellobiohydrolase variant of the invention may also comprise at least one of the following: a polypeptide product of a cellulose integrating protein, scaffoldin or a scaffoldin-like protein, for example CipA or CipC from Clostridium thermocellum or Clostridium cellulolyticum respectively. Scaffoldins and cellulose integrating proteins are multi-functional integrating subunits which may organize cellulolytic subunits into a multi-enzyme complex. This is accomplished by the interaction of two complementary classes of domain, i.e. a cohesion domain on scaffoldin and a dockerin domain on each enzymatic unit. The scaffoldin subunit also bears a cellulose-binding module that mediates attachment of the cellulosome to its substrate. A scaffoldin or cellulose integrating protein for the purposes of this invention may comprise one or both of such domains.

An enzyme mixture that comprises a cellobiohydrolase variant of the invention may also comprise at least one cellulose induced protein or modulating protein, for example as encoded by cip1 or cip2 gene or similar genes from Trichoderma reesei (see Foreman et al., J. Biol. Chem. 278(34), 31988-31997, 2003).

An enzyme mixture that comprises a cellobiohydrolase variant of the invention may comprise a member of each of the classes of the polypeptides described above, several members of one polypeptide class, or any combination of these polypeptide classes.

### Cellobiohydrolase Compositions

The cellobiohydrolase variants of the present invention may be used in combination with other optional ingredients such as a buffer, a surfactant, and/or a scouring agent. A buffer may be used with a cellobiohydrolase of the present invention (optionally combined with other cellulases, including another cellobiohydrolase) to maintain a desired pH within the solution in which the cellobiohydrolase is employed. The exact concentration of buffer employed will depend on several factors which the skilled artisan can determine. Suitable buffers are well known in the art. A surfactant may further be used in combination with the cellobiohydrolases of the present invention. Suitable surfactants include any surfactant compatible with the cellobiohydrolase and, optionally, with any other cellulases being used. Exemplary surfactants include an anionic, a non-ionic, and ampholytic surfactants. Suitable anionic surfactants include, but are not limited to, linear or branched alkylbenzenesulfonates; alkyl or alkenyl ether sulfates having linear or branched alkyl groups or alkenyl groups; alkyl or alkenyl sulfates; olefinsulfonates; alkanesulfonates, etc. Suitable counter ions for anionic surfactants include, but are not limited to, alkali metal ions such as sodium and potassium; alkaline earth metal ions such as calcium and magnesium; ammonium ion; and alkanolamines having 1 to 3 alkanol groups of carbon number 2 or 3. Ampholytic surfactants include, e.g., quaternary ammonium salt sulfonates, and betaine-type ampholytic surfactants. Nonionic surfactants generally comprise polyoxyalkylene ethers, as well as higher fatty acid alkanolamides or alkylene oxide adduct thereof, and fatty acid glycerine monoesters. Mixtures of surfactants can also be employed as is known in the art.

The cellobiohydrolase variants of the invention may be used at effective amounts, concentrations, and lengths of time. An effective amount of cellobiohydrolase is a concentration of cellobiohydrolase sufficient for its intended purpose. For example, an effective amount of cellobiohydrolase within a solution may vary depending on whether the intended purpose is to use the enzyme composition comprising the cellobiohydrolase in a saccharification process, or for example a textile application such as stone-washing denim jeans. The amount of cellobiohydrolase employed is further dependent on the equipment employed, the process parameters employed, and the cellulase activity, e.g., a particular solution will require a lower concentration of cellobiohydrolase where a more active cellulase composition is used as compared to a less active cellulase composition. A concentration of cellobiohydrolase and length of time that a cellobiohydrolase will be in contact with the desired target further depends on the particular use employed by one of skill in the art, as is described herein.

One skilled in the art may practice the present invention using cellobiohydrolases in either aqueous solutions, or a solid cellobiohydrolase concentrate. When aqueous solutions are employed, the cellobiohydrolase solution can easily be diluted to allow accurate concentrations. A concentrate can be in any form recognized in the art including, but not limited to, liquids, emulsions, gel, pastes, granules, powders, an agglomerate, or a solid disk. Other materials can also be used with or placed in the cellulase composition of the present invention as desired, including stones, pumice, fillers, solvents, enzyme activators, and anti-redeposition agents depending on the intended use of the composition.

### XI. Examples

The following examples are offered to illustrate, but not to limit the claimed invention.

### Example 1

### M. thermophila Wild-type CBH1a Gene Acquisition and Construction of Yeast Expression Vector

cDNA coding the secreted wild-type M. thermophila CBH1a protein (SEQ ID NO:2) was amplified from a cDNA library prepared by Symbio, Inc. (Menlo Park, Calif.). Expression constructs were prepared in which the CBH1a WT sequence was linked to its native (M. thermophila) signal peptide for secretion in S. cerevisiae. The signal peptide sequence was PCR amplified with cDNA from the cDNA library. The M. thermophila CBH1a cDNA construct was cloned into a ScINV1-pYTSec60 shuttle vector, so that transcription was under the control of a yeast TEF1 promoter, as known in the art. S. cerevisiae cells were transformed with the expression vector. Clones with correct CBH1a sequences were identified as the wild-type CBH1a DNA sequence (SEQ ID NO:1) and its encoded polypeptide (SEQ ID NO:2), using methods known in the art.

### Example 2

### Evaluation of M. thermophila Wild-Type CBH1a and Variants in Microtiter Plates

Yeast-produced M. thermophila wild-type CBH1a and Variant Nos. 1-472 were cloned and grown on agar plates containing 30 g/L glucose, 6.7 g/L yeast nitrogen base, 5 g/L ammonium sulfate, and 2 g/L amino acid drop-out mix minus uracil (D9535, United States Biological). Single colonies were picked and inoculated into 200 μl of synthetic media containing 30 g/L glucose, 6.7 g/L yeast nitrogen base, 5 g/L ammonium sulfate, and 2 g/L amino acid drop-out mix minus uracil (D9535, United States Biological, Swampscott, Mass.). Cells were grown overnight (at least 16 hours) in an incubator at 30° C. with shaking at 250 rpm. 20 μl of this culture was diluted into 380 μL of synthetic defined expression medium containing 30 g/L galactose, 6.7 g/L yeast nitrogen base without amino acids (Y0626, Sigma, St. Louis, Mo.), 5 g/L ammonium sulfate, 10 g/L potassium phosphate (monobasic), 24 g/L amino acid drop-out mix minus uracil (D9535, United States Biological), 3.6 ml TIPC solution (0.05 g/L thiamine, 0.1 g/L myo-inositol, 0.05 g/L calcium panthotenate and 1 g/L choline chloride), and 0.2 ml vitamin solution (1.5 g/L pyridoxine, 1 g/L P-amino butyric acid, 0.1 g/L biotin, 1 g/L riboflavin, 0.1 g/L folic acid and 1.5 g/L niacin). The pH of the medium was adjusted to pH 6 using sodium hydroxide. This culture was incubated for 48 hours at 37° C. while shaking at 250 rpm. The supernatants were harvested by centrifugation (4000 rpm, 4° C., 15 minutes).

The cellobiohydrolase activity of supernatants on biomass substrate (wheat straw pretreated under acidic conditions) for wild-type CBH1a or Variant Nos. 1-472 was measured by transferring 120 μL of supernatant into a reaction mixture containing 10 mg of biomass, 360 μL pre-treated filtrate (obtained by washing pretreated substrate solids with water) and 5 mg/L β-glucosidase in 128 mM sodium acetate pH 5 for a total volume of 500 pL. The microtiter plates were incubated at 55° C., with shaking at 950 rpm for 72 hours. After centrifugation of plates for 5 minutes at 4000 rpm, 20 μL of the 10× diluted reaction mixture was transferred into 180 μL of 2×GOPOD reagent (MEGAZYME, Ireland) and the reaction was incubated for 30 minutes at room temperature. The glucose produced from cellobiohydrolase activity of M. thermophila wild-type CBH1a or CBH1a variants on biomass was measured spectrophotometrically at 510 nm with a Spectramax M2 (Molecular Devices, Sunnyvale, Calif.). The glucose background was subtracted from data points using the negative control. Background negative control was obtained by using media supernatant from cultures of S. cerevisiae cells without the CBH1a gene in the plasmid. The amount of glucose was calculated based on the measured absorbance at 510 nm and using the standard curve.

Variant No. 473 was produced in M. thermophila as described in Example 3 below and assayed for cellobiohydroase activity on pretreated corn stover by measuring glucose production as described in Example 5 below.

Table 2 provides the relative cellobiohydrolase activity of CBH1a variants as compared to wild-type CBH1a (SEQ ID NO:2) for glucose production. Relative activity is presented as fold improvement over wild-type CBH1a for Variant Nos. 1-297; over CBH1a Variant No. 207 for Variant Nos. 298-471; over Variant No. 208 for Variant No. 472; and over Variant No. 472 for Variant No. 473. The glucose background was subtracted from data points. In Table 2, the amino acid substitutions listed for each variant correspond to residue positions of SEQ ID NO:2 (e.g., “Q46K” means that the residue at position 46 in SEQ ID NO:2 (glutamine) is substituted with lysine); the amino acid positions were determined by optimal alignment with SEQ ID NO:2.

TABLE 2
M. thermophila CBH1a Variant Polypeptides
and Relative Cellobiohydrolase Activity

| | | Relative Fold |
|---|---|---|
| | Amino acid | Improvement |
| Variant | Substitutions Relative | in Glucose |
| No. | to SEQ ID NO: 2 | Production |
| — | — | — |
| 1 | Q46K | + |
| 2 | S30T | ++ |
| 3 | L23E | ++ |
| 4 | S38H | + |
| 5 | A25K | + |
| 6 | Q46T | + |
| 7 | T32V | + |
| 8 | T24F | + |
| 9 | T24Q | + |
| 10 | T24L/G39D | ++ |
| 11 | Q46H/V362A | +++ |
| 12 | T24L | +++ |
| 13 | G39D | ++ |
| 14 | T61N/D62L | +++ |
| 15 | A25D/A64P | ++ |
| 16 | T58Y | ++ |
| 17 | R60T | ++ |
| 18 | S63A | ++ |
| 19 | T75L | +++ |
| 20 | A64M | + |
| 21 | T58V/V233I | + |
| 22 | S63D | ++ |
| 23 | A64D | + |
| 24 | Y77W | +++ |
| 25 | S76V | +++ |
| 26 | S83W | ++ |
| 27 | S96N | +++ |
| 28 | D94N | + |
| 29 | T103P | + |
| 30 | Y118D | ++ |
| 31 | K111E | + |
| 32 | Q117D | ++ |
| 33 | I122V | + |
| 34 | Q117E | ++ |
| 35 | Y118Q | + |
| 36 | Y118E | + |
| 37 | Y2H/K111Q | ++ |
| 38 | Y118G | + |
| 39 | Y118P | + |
| 40 | N109A | + |
| 41 | T120M | + |
| 42 | T120E | + |
| 43 | S107T | + |
| 44 | S119D | ++ |
| 45 | S177G | + |
| 46 | K180N | + |
| 47 | K180V | + |
| 48 | E208G | ++ |
| 49 | S177Q | + |
| 50 | Q211A | + |
| 51 | K180L | + |
| 52 | G178E | + |
| 53 | A169V/S177W | + |
| 54 | A169E | + |
| 55 | K180E | + |
| 56 | K175A | + |
| 57 | S177P | + |
| 58 | S177E | + |
| 59 | K175T | + |
| 60 | T221P | + |
| 61 | A315I | + |
| 62 | A219H | + |
| 63 | S212W | + |
| 64 | S212L | + |
| 65 | K223W | + |
| 66 | S212I | + |
| 67 | L311H | + |
| 68 | T221E | ++ |
| 69 | A315D | + |
| 70 | Y265N/L311R | + |
| 71 | K223A | ++ |
| 72 | K223L | ++ |
| 73 | S212Y | + |
| 74 | S319Q | + |
| 75 | N215E | + |
| 76 | K223D | + |
| 77 | A217N | + |
| 78 | K223G | ++ |
| 79 | A219N | + |
| 80 | A217E | ++ |
| 81 | K289Q | + |
| 82 | K289E | + |
| 83 | K289V | + |
| 84 | I273P | + |
| 85 | K294I | + |
| 86 | K294E | + |
| 87 | K294P | + |
| 88 | K289W | ++ |
| 89 | I273V | + |
| 90 | K289R | ++ |
| 91 | K289D | ++ |
| 92 | K294Q | ++ |
| 93 | S260Y | + |
| 94 | V250I | ++ |
| 95 | T267G | + |
| 96 | T267E | ++ |
| 97 | A271E | + |
| 98 | A271V | + |
| 99 | F243M/T267N | + |
| 100 | G252S | + |
| 101 | T267S | + |
| 102 | E257N | + |
| 103 | T267N | ++ |
| 104 | I251W | ++ |
| 105 | S260T | + |
| 106 | S260E | + |
| 107 | L311V | + |
| 108 | T221G | + |
| 109 | T214M | + |
| 110 | D216Q | + |
| 111 | D216V | + |
| 112 | Y2H/S314Q | + |
| 113 | A217Q | + |
| 114 | A219E | + |
| 115 | L311M | + |
| 116 | T338V/P464S | + |
| 117 | K330M | + |
| 118 | T338H | + |
| 119 | P340M | + |
| 120 | I339V | ++ |
| 121 | E336V | + |
| 122 | W351F | + |
| 123 | A358V | + |
| 124 | A357H | + |
| 125 | D353E | + |
| 126 | T348N | + |
| 127 | R354L | ++ |
| 128 | A357V | ++ |
| 129 | D350A | ++ |
| 130 | D350Q | + |
| 131 | D353A | + |
| 132 | A357G | + |
| 133 | A358S | + |
| 134 | Q366S | + |
| 135 | V372S | + |
| 136 | M374L | + |
| 137 | V372A | + |
| 138 | V372M | + |
| 139 | V362P | ++ |
| 140 | V372P | + |
| 141 | V372Q | + |
| 142 | D367N | + |
| 143 | D367S | + |
| 144 | A379K/H392D | + |
| 145 | I405R | + |
| 146 | H392S | + |
| 147 | H392E | + |
| 148 | H392P | ++ |
| 149 | H392N | + |
| 150 | H392S/G487D | ++ |
| 151 | A379E | ++ |
| 152 | H392Q | + |
| 153 | H392P/I405Y | + |
| 154 | Y2H/A217T/H392V | + |
| 155 | H392A | ++ |
| 156 | H392E/K517E | ++ |
| 157 | N202D/H392A | + |
| 158 | P82T/H392P | + |
| 159 | H392R | + |
| 160 | H392T | + |
| 161 | P433L | + |
| 162 | S452E | ++ |
| 163 | A430F | + |
| 164 | G453L | ++ |
| 165 | P433S | ++ |
| 166 | N434E | ++ |
| 167 | V394A | +++ |
| 168 | T420M/S452Y | + |
| 169 | A432D | + |
| 170 | A430S | + |
| 171 | N434S | + |
| 172 | V451L | + |
| 173 | G453H | + |
| 174 | A408V | + |
| 175 | T420S | ++ |
| 176 | E427N | ++ |
| 177 | V424R | ++ |
| 178 | V428L | ++ |
| 179 | A408S | ++ |
| 180 | A417S | +++ |
| 181 | A426S | ++ |
| 182 | A408D | +++ |
| 183 | V451L/S452E | + |
| 184 | I405R/A408D | + |
| 185 | Y2H | + |
| 186 | V451Y | + |
| 187 | T450I | + |
| 188 | E69T/I122V/V362T/V394A | + |
| 189 | T24R | + |
| 190 | T24N/S97G/Q349T/D350E/V394D | + |
| 191 | T24N/P82E/S83T/V394D | + |
| 192 | T24V/P82E | + |
| 193 | T24N/Q349T/W351Y | + |
| 194 | T24K/K294P/Q349T/D350E/W351Y | + |
| 195 | T24V/V394D | + |
| 196 | Q349T/D350E/W351Y | + |
| 197 | T24N/T126V/A379E | + |
| 198 | T24N | + |
| 199 | T24R/S96E/S97G/T126V/D350E/A379E/ | + |
| | V394D | |
| 200 | T24V/D350E/W351Y | + |
| 201 | T24K/V394D | + |
| 202 | T24V/P82E/S97G | + |
| 203 | Q349T/W351Y | + |
| 204 | T24V/S97G/Q349T/V394D | ++ |
| 205 | T24V | + |
| 206 | T24V/S96E/S97G/D350E/W351Y | + |
| 207 | T24N/A379E/V394D | ++ |
| 208 | T24N/D350E/W351Y/V394D | ++ |
| 209 | T24N/S83T/S96E/S97G/V394D | ++ |
| 210 | T24N/A379E/V394D | + |
| 211 | T24V/S97G/D350E | + |
| 212 | T24V/D350E | + |
| 213 | V45Q/Q46N/Y118H/K289H/T290S/T363D/ | + |
| | V394A | |
| 214 | T24F/S44T/S86Q/S177P/T290S/T363D | + |
| 215 | S86Q/K303P/I304F/V362T/T363D/A393Y/ | + |
| | V394A/P471S/V472K | |
| 216 | T24I/S44T/Q46N/S86Q/K87N/Y118H/ | + |
| | P471S/V472K | |
| 217 | S44T/K289H/T363D/V394A/V472K | + |
| 218 | T24I/S44T/Q46N/K289H/V472K | + |
| 219 | T290S/I304F/V362T/V394A/P471S | + |
| 220 | V394A/V472K | + |
| 221 | P471S | + |
| 222 | E208K/K289H/V362T/V394A/P471S/V472K | + |
| 223 | Q46N/T363D/V394A | + |
| 224 | Q46N/S86Q/S177P | + |
| 225 | T24I/S177P/K289H/V362T/S401D/V472K | + |
| 226 | K87N/Y118H/T290S/T363D/P471S | + |
| 227 | S86Q/T290S/I304F/V362T/V394A/P471S | + |
| 228 | P471S/V472K | + |
| 229 | T24I/S44T/Q46N/K289H/I304F/A393Y/ | + |
| | P471S | |
| 230 | S401N/P471S | + |
| 231 | T24I/T290S/I304F | + |
| 232 | T221E/E427N | + |
| 233 | T221E/E427N | + |
| 234 | S177E/T221E | + |
| 235 | S177E/T221E | + |
| 236 | A64D/S177E/T221G | + |
| 237 | A64D/S177E/T221G | + |
| 238 | A64T/S177E | + |
| 239 | T24L/S177E | + |
| 240 | T24L/S177E | + |
| 241 | V12G | + |
| 242 | T221E/E427N | + |
| 243 | S177E/T221E/E336V | + |
| 244 | S177E/T221E/E336V | + |
| 245 | V362A | + |
| 246 | T24L/T221E | + |
| 247 | T24Q/S177E/K294I/E427N | + |
| 248 | T24M/S177E | + |
| 249 | T24L/S177E | + |
| 250 | A64D | + |
| 251 | T221E/E427N | + |
| 252 | T221E/E427N | + |
| 253 | T221E/E427N | + |
| 254 | S177E | + |
| 255 | S177E/E427N | + |
| 256 | S177E/E427N | + |
| 257 | T221E | + |
| 258 | Q46H/S177E | + |
| 259 | Q46T/A357V/V394A | + |
| 260 | T267E/V362P/T363N/V428L/S452E | + |
| 261 | S30T | + |
| 262 | Y77W/T267N/V362P/V424R | + |
| 263 | S30T/D275N/V362P/T363N/V451Y | ++ |
| 264 | S30T/T363N/A408D/V424R/A426S | + |
| 265 | A408D/V428L | + |
| 266 | A6P/A9P/V12G/W57G | + |
| 267 | S30T/V362P/V428L | + |
| 268 | T267N/A408D | + |
| 269 | S30T/Y77W/E208K/V362P/T363N/S452E | + |
| 270 | L8R/V12A/S30T/W33G/T75L/Y77W/ | + |
| | T267N/T363N/I405R/A426S/S452E | |
| 271 | S30T/V362P/T363N/A408D/V428L | + |
| 272 | T267N/V451Y | + |
| 273 | D216N/H392S/V394A/W403Y | ++ |
| 274 | T58V/A64T/E69T | + |
| 275 | E69T | + |
| 276 | H392S/V394A | + |
| 277 | H392A/W403Y | + |
| 278 | H392A/V394A/W403Y | + |
| 279 | H392S/V394A | ++ |
| 280 | E208K/V362T/V394A/W403Y | + |
| 281 | A64T | + |
| 282 | V362T/H392S | ++ |
| 283 | A64T/A219T | + |
| 284 | V394A/W403Y | ++ |
| 285 | A64T/H392S/W403Y | + |
| 286 | T58V/A64T/E69T/A219T/V362T/V394A | + |
| 287 | T58V | + |
| 288 | A64T | + |
| 289 | T75L/S76V/H392S | ++ |
| 290 | Y77W | ++ |
| 291 | Y77W | ++ |
| 292 | T75L/Y77W | ++ |
| 293 | H392S | ++ |
| 294 | K517E | + |
| 295 | S76V/Y77W | + |
| 296 | S79P/S96E/S97G/Q117N/G155P/S177P/ | + |
| | S212P/K294P/E343P/V362T/T363D/P381G/ | |
| | A417P | |
| 297 | S79P/Q117N/G155P/S177P/S212P/K294P/ | + |
| | E343P/P381G/A417P/P455D/G458D | |
| 298 | T24N/A25D/Y118D/S177E/A217E/K223L/ | ** |
| | A379E/V394D | |
| 299 | T24N/Y118D/S177E/A217E/K289E/P340M/ | * |
| | A379E/V394D | |
| 300 | T24N/S177E/A217E/K223G/A379E/V394D | * |
| 301 | T24N/A25D/A64M/A217E/A379E/V394D | * |
| 302 | T24N/Y118D/A217E/P340M/A379E/V394D | * |
| 303 | T24N/Y118D/S177E/K289W/A379E/V394D | * |
| 304 | T24N/A25D/A217E/A379E/V394D | * |
| 305 | T24N/A25D/S177E/A379E/V394D | * |
| 306 | T24N/A217E/K289W/P340M/A379E/V394D | * |
| 307 | T24N/A25D/A64P/K223W/A379E/V394D | * |
| 308 | T24N/A64P/Y118D/K223W/K289R/A379E/ | ** |
| | V394D | |
| 309 | T24N/Y118D/S177E/A217E/K223G/A379E/ | * |
| | V394D | |
| 310 | T24N/A25D/Y118D/A379E/V394D/T420S | * |
| 311 | T24N/S177E/A217E/P340M/A379E/V394D/ | * |
| | T420S | |
| 312 | T24N/S177E/A217E/K223D/A379E/V394D | * |
| 313 | T24N/A64M/Y118D/S177E/K223L/A379E/ | * |
| | V394D | |
| 314 | T24N/A25D/Y118D/S177E/K289D/A379E/ | * |
| | V394D | |
| 315 | T24N/A217E/K223L/K289E/P340M/A379E/ | * |
| | V394D | |
| 316 | T24N/P340M/A379E/V394D | * |
| 317 | T24N/K223D/A379E/V394D/T420S | * |
| 318 | T24N/S177E/K223W/A379E/V394D | * |
| 319 | T24N/A25D/A64P/Y118D/A379E/V394D | * |
| 320 | T24N/A25D/A64P/S177E/A217E/K223W/ | * |
| | A379E/V394D/T420S | |
| 321 | T24N/A25D/Y118D/K223D/P340M/A379E/ | * |
| | V394D | |
| 322 | T24N/S177E/K289E/A379E/V394D | ** |
| 323 | T24N/A25D/S177E/P340M/A379E/V394D/ | * |
| | T420S | |
| 324 | T24N/Y118D/A217E/K289W/A379E/V394D | * |
| 325 | T24N/S177E/A217E/K223L/A379E/V394D | * |
| 326 | T24N/A25D/K223L/P340M/A379E/V394D | * |
| 327 | T24N/A217E/K289E/A379E/V394D | * |
| 328 | T24V/V394D | * |
| 329 | T24N/D350E/W351Y/V394D | * |
| 330 | D350E/W351Y/A379E/V394D | * |
| 331 | V394D | * |
| 332 | V12G/T24V/T221E/V394D | * |
| 333 | M1L/Y2S/T24N/D350E/W351Y/V394D | * |
| 334 | A379E/V394D | * |
| 335 | T24N/S83T/S96E/S97G/M137V/V394D | * |
| 336 | T24V/S177E/D350E/W351Y/V394D | * |
| 337 | T24N/V394D | * |
| 338 | T24N/S83T/S96E/S97G/A379E/V394D | * |
| 339 | T24N/A379E/V394D | * |
| 340 | T24N/A64T/A379E/V394D | * |
| 341 | T24N/A64T/S76V/A379E/H392A/V394D/ | * |
| | K517E | |
| 342 | T24N/A64T/T75L/A379E/H392A/V394D/ | * |
| | K517E | |
| 343 | T24N/A64T/T75L/A379E/H392E/V394D/ | * |
| | K517E | |
| 344 | T24N/Y77W/A379E/V394D/K517E | * |
| 345 | T24N/A64T/Y77W/A379E/H392S/V394D/ | * |
| | K517E | |
| 346 | T24N/A64T/A379E/V394D/K517E/D520N | * |
| 347 | T24N/A64T/S76V/A379E/H392S/V394D/ | * |
| | K517E | |
| 348 | T24N/A64T/A379E/V394D/K517E | * |
| 349 | T24N/S83T/S97G/S177E/A379E/V394D | * |
| 350 | T24N/S177E/A379E/V394D/E427N | * |
| 351 | T24V/S96E/S177E/W351Y/A379E/V394D/ | * |
| | E427N | |
| 352 | T24N/S96E/S177E/D350E/W351Y/A379E/ | * |
| | V394D | |
| 353 | T24V/S96E/S177E/D350E/A379E/V394D | * |
| 354 | T24N/S83T/S96E/W351Y/A379E/V394D | * |
| 355 | T24N/S97G/S177E/W351Y/A379E/V394D/ | * |
| | E427N | |
| 356 | T24V/D350E/A379E/V394D | * |
| 357 | V12G/T24N/W351Y/A379E/V394D/E427N | * |
| 358 | T24N/T221E/W351Y/A379E/V394D | * |
| 359 | T24Q/S83T/S177E/T221E/A379E/V394D/ | * |
| | E427N | |
| 360 | T24V/S96E/T221E/D350E/W351Y/A379E/ | * |
| | V394D/E427N | |
| 361 | T24N/S177E/W351Y/A379E/V394D/E427N | * |
| 362 | T24N/S83T/S177E/A379E/V394D | * |
| 363 | T24V/S97G/D350E/W351Y/A379E/V394D | * |
| 364 | T24V/S97G/T221E/D350E/A379E/V394D | * |
| 365 | T24N/S177E/A379E/V394D/E427N/N463K | * |
| 366 | T24N/S177E/W351Y/A379E/V394D | * |
| 367 | T24V/A379E/V394D | * |
| 368 | T24N/A379E/V394D/Y513G | *** |
| 369 | T24N/A379E/V394D/G502I | *** |
| 370 | T24N/A379E/V394D/P506A | *** |
| 371 | T24N/A379E/V394D/Q508W | ** |
| 372 | T24N/A379E/V394D/Y513W | *** |
| 373 | T24N/A379E/V394D/K517P | *** |
| 374 | T24N/A379E/V394D/Q524W | *** |
| 375 | T24N/A379E/V394D/H494K | ** |
| 376 | T24N/A379E/V394D/I501W | ** |
| 377 | T24N/A379E/V394D/T516K | *** |
| 378 | T24N/A379E/V394D/S523A | *** |
| 379 | T24N/A379E/V394D/G500E | ** |
| 380 | T24N/G178S/A379E/V394D/A492V | *** |
| 381 | T24N/A379E/V394D/G505E | *** |
| 382 | T24N/A379E/V394D/L518A | ** |
| 383 | T24N/A379E/V394D/H494W | ** |
| 384 | T24N/G222S/A379E/V394D/E510C | *** |
| 385 | T24N/A379E/V394D/W521A | *** |
| 386 | Y2H/T24N/A379E/V394D/E496L | ** |
| 387 | T24N/A379E/V394D/P506L | ** |
| 388 | T24N/A379E/V394D/P512R | ** |
| 389 | T24N/A379E/V394D/Y495G | *** |
| 390 | T24N/A379E/V394D/A492C/L526E | ** |
| 391 | T24N/A379E/V394D/I501S | *** |
| 392 | T24N/A379E/V394D/L526F | ** |
| 393 | T24N/A379E/V394D/T504G | *** |
| 394 | T24N/A379E/V394D/L518E | *** |
| 395 | T24N/A379E/V394D/P506V | *** |
| 396 | T24N/A379E/V394D/V491A | *** |
| 397 | T24N/A379E/V394D/G502L | *** |
| 398 | T24N/A379E/V394D/E496R | ** |
| 399 | T24N/A379E/V394D/Q524P | *** |
| 400 | T24N/A379E/V394D/T516R | ** |
| 401 | Y2H/T24N/A379E/V394D/G409D/T504A | ** |
| 402 | T24N/A379E/V394D/E510R | *** |
| 403 | T24N/A379E/V394D/L526P | *** |
| 404 | T24N/A379E/V394D/G502Q | ** |
| 405 | T24N/A379E/V394D/G505P | *** |
| 406 | T24N/A379E/V394D/P512A | *** |
| 407 | T24N/A379E/V394D/K517G | *** |
| 408 | T24N/A379E/V394D/G500R | *** |
| 409 | T24N/A379E/V394D/N519H | ** |
| 410 | T24N/A379E/V394D/T507W | *** |
| 411 | T24N/A379E/V394D/E496W | *** |
| 412 | T24N/A379E/V394D/Q524G | *** |
| 413 | T24N/A379E/V394D/H494E | *** |
| 414 | T24N/A379E/V394D/H494A | ** |
| 415 | Y2H/T24N/A379E/V394D/V491P | *** |
| 416 | T24N/A379E/V394D/P506H | *** |
| 417 | T24N/A379E/V394D/T516A | *** |
| 418 | T24N/A379E/V394D/H494R/T504M | *** |
| 419 | T24N/A379E/V394D/S523W | ** |
| 420 | T24N/A379E/V394D/S511W | ** |
| 421 | T24N/A379E/V394D/S511P | ** |
| 422 | T24N/A379E/V394D/G481W/G500V | ** |
| 423 | T24N/A379E/V394D/T486A | *** |
| 424 | T24N/A379E/V394D/S469C | *** |
| 425 | T24N/D268Y/A379E/V394D/V466A | *** |
| 426 | T24N/A379E/V394D/P455W | *** |
| 427 | T24N/A379E/V394D/G481F | *** |
| 428 | T24N/A379E/V394D/T470D | *** |
| 429 | T24N/A271V/A379E/V394D/S479K | *** |
| 430 | T24N/A379E/V394D/G490A | *** |
| 431 | T24N/A379E/V394D/P473L | *** |
| 432 | T24N/A379E/V394D/S476P | ** |
| 433 | T24N/A379E/V394D/G481P | ** |
| 434 | T24N/A379E/V394D/G490W | *** |
| 435 | T24N/D367Y/A379E/V394D/G457R | *** |
| 436 | T24N/A379E/V394D/P465A | *** |
| 437 | T24N/A379E/V394D/P465M | ** |
| 438 | T24N/A379E/V394D/S475R | *** |
| 439 | T24N/D268Y/A379E/V394D/P473W | *** |
| 440 | T24N/A379E/V394D/S474D | *** |
| 441 | T24N/A379E/V394D/P455F | *** |
| 442 | T24N/A379E/V394D/T478K | *** |
| 443 | T24N/A379E/V394D/T478W | ** |
| 444 | T24N/A379E/V394D/G458A | ** |
| 445 | T24N/A379E/V394D/S480M | ** |
| 446 | T24N/A379E/V394D/P464R | ** |
| 447 | T24N/A379E/V394D/G487I | ** |
| 448 | T24N/A379E/V394D/G484L | *** |
| 449 | T24N/A379E/V394D/G484P | ** |
| 450 | T24N/A379E/V394D/G457R | ** |
| 451 | T24N/K115N/A379E/V394D/S467R | *** |
| 452 | T24N/A379E/V394D/S459E | *** |
| 453 | T24N/A379E/V394D/S475L | ** |
| 454 | T24N/A379E/V394D/V472A | *** |
| 455 | T24N/A379E/V394D/T486K | ** |
| 456 | T24N/A379E/V394D/S475F | ** |
| 457 | T24N/A379E/V394D/P485W | *** |
| 458 | T24N/A379E/V394D/D456R | *** |
| 459 | T24N/A379E/V394D/S475W | *** |
| 460 | T24N/A379E/V394D/P465R | *** |
| 461 | T24N/A379E/V394D/V466W | *** |
| 462 | T24N/A379E/V394D/P473T | ** |
| 463 | T24N/A379E/V394D/P455L | *** |
| 464 | T24N/A379E/V394D/G481W | ** |
| 465 | T24N/A379E/V394D/P455Q | *** |
| 466 | T24N/A379E/V394D/S482G | ** |
| 467 | T24N/A379E/V394D/T489Y | ** |
| 468 | T24N/A379E/V394D/T477V/ | ** |
| 469 | T24N/A379E/V394D/G481R | ** |
| 470 | T24N/E343D/A379E/V394D/S483G | ** |
| 471 | T24N/A379E/V394D/T470E | ** |
| 472 | T24N/T58V/D350E/W351Y/T363D/V394D/ | °° |
| | V451Y/I501Q | |
| 473 | T58V/D350E/W351Y/T363D/V394D/V451Y/ | # |
| | I501Q | |

+ = 1.0 to 1.2 fold improvement over wild-type M. thermophila CBH1a
++ = 1.3 to 1.5 fold improvement over wild-type M. thermophila CBH1a
+++ >1.5 fold improvement over wild-type M. thermophila CBH1a
* = 1.0 to 1.2 fold improvement over Variant No. 207
** = 1.3 to 1.5 fold improvement over Variant No. 207
*** >1.5 fold improvement over Variant No. 207
°° 1.3 to 1.5 fold improvement over Variant No. 208
# = 1.0 to 1.2 fold improvement over Variant No. 472

### Example 3

### Production of CBH1a Variants in the M. thermophila Host

A two-step fermentation process (inoculation and main fermentations starting from spores) was used to express M. thermophila CBH1a variant genes in M. thermophila. Plasmids containing genes encoding M. thermophila CBH1a Variant No. 24, 207, 208, 251, 472, or 473 were transformed into a M. thermophila strain and plated on agar plates containing M3-01 medium with 22.93% sucrose (ingredients of M3-01 Medium: 6.0 g/L Sodium Nitrate, 0.52 g/L Potassium Chloride, 1.52 g/L Potassium Phosphate monobasic (KH₂PO₄), 0.24 g/L Magnesium Sulfate, 1.6 mg/L Copper(II) Sulfate pentahydrate (CuSO₄5H₂O), 5 mg/L Ferrous Sulfate heptahydrate (FeSO₄7H₂O), 22 mg/L Zinc Sulfate heptahydrate (ZnSO₄7H₂O), 5 mg/L Manganese(II) Chloride tetrahydrate (MnCl₂4H₂O), 1.8 mg/L Cobalt(II) Sulfate heptahydrate (CoSO₄7H₂O), 1.5 mg/L Sodium Molybdate dihydrate (Na₂MoO₄2H₂O), 11 mg/L Boric Acid, 50 mg/L EDTA, 10.0 g/L Glucose, 1.0 g/L CAS amino acids (Tritium Microbiologie B. V., The Netherlands), 16 g/L agar, 1 ml/L 1000× Pen/Strep after sterilization (1000× Pen/Strep: 2 g Penicillin G and 5 g Streptomycin dissolved in 100 ml H₂O, sterilized by filtration). The pH of the medium was adjusted to 6.5 with 10 M KOH and autoclaved for 25 minutes at 121° C. The plates were incubated at 35° C. for 5 days.

Spores harvested from the agar plates were used to inoculate a 100 mL F1-01 inoculum medium sterilized in a 500 mL Erlenmeyer flask to reach 5×10⁴-10⁵ spores/mL initial spore number (Ingredients of F1-01 Inoculum Medium: 0.50 g/L Potassium Phosphate dibasic (K2HPO₄), 0.05 g/L Potassium Chloride, 0.007 g/L Ferrous Sulfate heptahydrate (FeSO₄7H₂O), 1.00 g/L Yeast Extract (only KAT), 10 g/L Pharmamedia (Traders Protein, Lubbock, Tex., USA), 10 g/L D(+)Lactose monohydrate, 10 g/L Glucose after sterilization, 1 ml/L 1000× Pen/Strep after sterilization (1000× Pen/Strep: 2 g Penicillin G and 5 g Streptomycin dissolved in 100 ml H₂O, sterilized by filtration). The pH of the medium was adjusted to 7.0 with 10 M NaOH and autoclaved for 25 minutes at 121° C. To prepare the inoculum culture, the flask was incubated at 35° C., 85% humidity for 3 days with shaking at 250 rpm and 25 mm displacement. 15 mL F1-01 Main Fermentation Medium sterilized in a 100 mL Erlenmeyer flask was inoculated with 750 μL of the obtained inoculum culture (ingredients of F1-01 Main Fermentation Medium: 0.66 g/L Potassium Phosphate dibasic (K2HPO₄), 0.24 g/L Potassium Phosphate monobasic (KH₂PO₄), 8.00 g/L Ammonium Sulphate, 12.00 g/L Sodium Citrate tribasic dehydrate, 0.15 g/L Yeast Extract (only KAT), 0.09 g/L Magnesium Sulfate heptahydrate, 0.80 g/L Calcium Chloride dihydrate, 24.80 g/L Pharmamedia (Traders Protein, Lubbock, Tex., USA), 26.40 g/L D(+)Lactose monohydrate, 64.80 g/L Cellulose (AlphaCel BH200A)). The medium was autoclaved for 25 minutes at 121° C. The main fermentation was carried out by incubation at 35° C., 85% humidity for 6 days with shaking at 300 rpm and 25 mm displacement. After finishing the main fermentation the cells were pelleted by centrifugation (4500 rpm, 15 min, 4° C.). The clear medium supernatant containing the secreted M. thermophila CBH1a enzyme was collected and stored at −20° C. until used.

### Example 4

### Evaluation of M. thermophila Wild-Type CBH1a and a Subset of Variants

Wild-type M. thermophila CBH1a and Variant Nos. 24, 207, 208, 251, and 472 were assayed for cellobiohydroase activity on pretreated wheat straw by measuring glucose production. Wild-type CBH1a and Variant Nos. 24, 207, 208, 251, and 472 proteins were purified from the fermentation broth of M. thermophila cultures and the proteins were quantified using a HPLC method of protein quantification and/or a bicinchoninic acid (BCA) protein assay, as known in the art. The proteins were then adjusted to the same concentration and equal amounts of protein were used in each glucose production reaction. CBH1a activity was assayed according to the conditions as described in Example 2. Under the conditions tested, CBH1a Variant Nos. 24, 207, 208, 251, and 472 showed up to −20% improvement in the amount of glucose produced as compared to wild-type CBH1a.

### Example 5

### Evaluation of CBH1a Variants

CBH1a Variant Nos. 472 and 473 were assayed for cellobiohydroase activity on pretreated corn stover by measuring glucose production. The protein concentration of fermentation broth of M. thermophila containing Variant No. 472 or 473 was quantified by Bradford assay and/or a bicinchoninic acid (BCA) protein assay, as known in the art. The fermentation broths containing the variants were then adjusted to the same concentration of protein and equal amounts of protein were used in each glucose production reaction. The cellobiohydrolase activity was measured in a reaction mixture containing 40 mg of pretreated corn stover, 0.4% total protein broth with respect to glucan concentration, and 0.081% β-glucosidase in 128 mM sodium acetate pH 5. Glucose production was measured as described in Example 2. Under the conditions tested, CBH1a Variant No. 473 exhibited about a 1.0- to 1.2-fold improvement in glucose production over CBH1a Variant No. 472.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A recombinant cellobiohydrolase type 1a (CBH1a) variant comprising at least about 70% sequence identity to SEQ ID NO:2 and comprising an amino acid substitution, relative to SEQ ID NO:2, at one or more positions selected from 351, 363, 58, 451, 501, 394, 350, and 24, wherein the positions are numbered with reference to SEQ ID NO:2.

2. The variant of claim 1, comprising an amino acid substitution, relative to SEQ ID NO:2, at one or more positions selected from 350, 351, and 394, wherein the positions are numbered with reference to SEQ ID NO:2.

3. The variant of claim 2, wherein the amino acid residue at position 350 is glutamic acid (X350E); the amino acid residue at position 351 is tyrosine (X351Y); and/or the amino acid residue at position 394 is aspartic acid (X394D).

4. The variant of claim 1, comprising an amino acid substitution, relative to SEQ ID NO:2, at one or more positions selected from 24, 58, 363, 451, and 501, wherein the positions are numbered with reference to SEQ ID NO:2.

5. The variant of claim 4, wherein the amino acid residue at position 24 is asparagine (X24N); the amino acid residue at position 58 is valine (X58V); the amino acid residue at position 363 is aspartic acid (X363D); the amino acid residue at position 451 is tyrosine (X451Y); and/or the amino acid residue at position 501 is glutamine (X501Q).

6. The variant of claim 1, wherein the variant is derived from a Myceliophthora thermophila cellobiohydrolase.

7. (canceled)

8. The variant of claim 1, that has an improved property relative to wild-type Myceliophthora thermophila CBH1a (SEQ ID NO:2).

9. The variant of claim 8, wherein the variant has increased cellobiohydrolase activity after incubation at pH 5.0 and 55° C. for 72 hours as compared to wild-type Myceliophthora thermophila CBH1a (SEQ ID NO:2).

10. A recombinant polynucleotide encoding the variant of claim 1.

11. A recombinant host cell expressing a cellobiohydrolase variant having the amino acid sequence of a polypeptide of claim 1.

12. An enzyme composition comprising the variant cellobiohydrolase polypeptide of claim 1.

13. A recombinant cellobiohydrolase type 1a (CBH1a) variant comprising at least about 70% sequence identity to SEQ ID NO:2 and comprising an amino acid substitution, relative to SEQ ID NO:2, at one or more positions selected from Y2, L23, T24, A25, S30, G39, Q46, T58, R60, T61, S62, S63, A64, T75, S76, Y77, S83, S96, S97, K111, K115, Q117, Y118, S119, S177, G178, E208, D216, A217, T221, G222, K223, V250, I251, T267, D268, A271, D275, K289, K294, I339, E343, Q349, D350, W351, R354, A357, V362, T363, D367, A379, H392, V394, W403, A408, G409, A417, V424, A426, E427, V428, P433, N434, V451, S452, G453, P455, D456, G457, G458, S459, P464, P465, V466, S467, S469, T470, V472, P473, S474, S475, S476, T477, T478, S479, S480, G481, S482, S483, G484, P485, T486, G487, T489, G490, V491, A492, H494, Y495, E496, G500, I501, G502, T504, G505, P506, T507, Q508, T510, S511, P512, Y513, T516, K517, L518, N519, W521, S523, Q524, and L526, wherein the positions are numbered with reference to SEQ ID NO:2.

14. The variant of claim 13 that has increased thermoactivity or specific activity as compared to wild-type Myceliophthora thermophila CBH1a (SEQ ID NO:2).

15. The variant of claim 13, wherein the variant comprises one or more amino acid substitutions selected from Y2H, L23E, T24L/N/V, A25D, 530T, G39D, Q46H, T58V/Y, R60T, T61N, S62L, 563A/D, A64P, T75L, S76V, Y77W, S83T/W, S96E/N, S97G, K111Q, K115N, Q117D/E, Y118D, S119D, S177E, G1785, E208G, D216N, A217E, T221E, G222S, K223A/G/L/W, V2501,1251W, T267E/N, D268Y, A271V, D275N, K289D/E/R/W, K294Q, I339V, E343D, Q349T, D350A/E, W351Y, R354L, A357V, V362A/P/T, T363N, D367Y, A379E, H392A/E/P/S, V394A/D, W403Y, A408D/S, G409D, A417S, V424R, A426S, E427N, V428L, P433S, N434E, V451Y, S452E, G453L, P455F/L/Q/W, D456R, G457R, G458A, S459E, P464R, P465A/M/R, V466A/W, S467R, S469C, T470D/E, V472A, P473L/T/W, S474D, S475F/L/R/W, S476P, T477V, T478K/W, S479K, S480M, G481F/P/R/W, S482G, S483G, G484L/P, P485W, T486A/K, G487D/I, T489Y, G490A/W, V491A/P, A492C/V, H494A/E/K/R/W, Y495G, E496L/R/W, G500E/R/V, 1501Q/S/W, G5021/L/Q, T504A/G/M, G505E/P, P506A/H/L/V, T507W, Q508W, T510R, S511P/W, P512A/R, Y513G/W, T516A/K/R, K517E/G/P, L518A/E, N519H, W521A, S523A/W, Q524G/P/W, and/or L526E/F/P.

16. (canceled)

17. The variant of claim 13, comprising an amino acid substitution at one or more positions selected from T24, T58, V451, D350, W351, T363 A379, V394, and I501.

18-20. (canceled)

21. The variant of claim 13, comprising an amino acid substitution at position Y77.

22. The variant of claim 21, comprising the amino acid substitution Y77W.

23. (canceled)

24. The variant of claim 17, comprising one or more amino acid substitutions selected from T24N, T58V, D350E, W351Y, T363D, V394D, V451Y, and I501Q.

25. (canceled)

26. The variant of claim 17, comprising one or more amino acid substitutions selected from T58V, D350E, W351Y, T363D, V394D, V451Y, and I501Q.

27. The variant of claim 13, having the amino acid sequence of any of SEQ ID NOs:18-23.

28. The variant of claim 13, wherein the variant exhibits at least a 1.1-fold increase in thermoactivity or specific activity relative to wild-type Myceliophthora thermophila CBH1a (SEQ ID NO:2).

29. (canceled)

30. The variant of claim 13, wherein the variant has increased activity after incubation at pH 5.0 and 55° C. for 72 hours as compared to wild-type Myceliophthora thermophila CBH1a (SEQ ID NO:2).

31. A cellobiohydrolase type 1a (CBH 1a) variant comprising at least 70% sequence identity to SEQ ID NO:2 and comprising one or more amino acid substitutions selected from X2H, X23E, X24L/N/V, X25D, X30T, X39D, X46H, X58Y, X60T, X61N, X62L, X63A/D, X64P, X75L, X76V, X77W, X83T/W, X96E/N, X97G, X111Q, X115N, X117D/E, X118D, X119D, X177E, X178S, X208G, X216N, X217E, X221E, X222S, X223A/G/L/W, X250I, X251W, X267E, X268Y, X271V, X275N, X289D/E/R/W, X294Q, X339V, X343D, X349T, X350A, X351Y, X354L, X357V, X362A/P/T, X363N, X367Y, X379E, X392E/P/S, X394A/D, X403Y, X408D/S, X409D, X417S, X424R, X426S, X427N, X428L, X433S, X434E, X451Y, X452E, X453L, X455F/L/Q/W, X456R, X457R, X458A, X459E, X464R, X465A/M/R, X466A/W, X467R, X469C, X470D/E, X472A, X473L/T/W, X474D, X475F/L/R/W, X476P, X477V, X478K/W, X479K, X480M, X481F/P/R/W, X482G, X483G, X484L/P, X485W, X486A, X487D/I, X489Y, X490A/W, X491A/P, X492C/V, X494A/E/K/R/W, X495G, X496L/R/W, X500E/R/V, X501Q/S/W, X502I/L/Q, X504A/G/M, X505E/P, X506A/H/L/V, X507W, X508W, X510R, X511P/W, X512A, X513G/W, X516A/K/R, X517E/G/P, X518A/E, X519H, X521A, X523A/W, X524G/P/W, and X526E/F/P, wherein the position is numbered with reference to SEQ ID NO:2, and wherein the cellobiohydrolase variant has increased thermoactivity or specific activity in comparison to secreted wild-type CBH1a (SEQ ID NO:2).

32. The variant of claim 31, wherein the variant comprises one or more amino acid substitution sets as set forth in Table 2.

33-35. (canceled)

36. The variant of claims 31, wherein the variant exhibits at least a 1.1-fold increase in thermoactivity or specific activity relative to the wild-type cellobiohydrolase from which the variant is derived.

37. A recombinant polynucleotide encoding the variant of claim 13.

38. An expression vector comprising the recombinant polynucleotide of claim 10.

39. A recombinant host cell comprising the expression vector of claim 38.

40. A recombinant host cell expressing a cellobiohydrolase variant having the amino acid sequence of a polypeptide of claim 13.

41. The recombinant host cell of claim 39, wherein the cell is a eukaryotic cell.

42. (canceled)

43. (canceled)

44. The recombinant host cell of claim 39, wherein the cell further produces at least one additional cellulase enzyme.

45. (canceled)

46. An enzyme composition comprising the cellobiohydrolase variant of claim 13.

47. The enzyme composition of claim 12, further comprising at least one additional cellulase enzyme.

48. A method of producing a cellobiohydrolase type 1a (CBH1a) variant comprising culturing the host cell of claim 11, under conditions in which the CBH1a variant protein is produced by the cell.

49. (canceled)

50. A method of producing a fermentable sugar from a cellulosic substrate, comprising contacting the cellulosic substrate with a β-glucosidase (BgI), an endoglucanase (EG), a type 2b cellobiohydrolase (CBH2b), a glycoside hydrolase 61(GH61), and the CBH1a variant of claim 1, under conditions in which the fermentable sugar is produced.

51. A method of producing an end-product, the method comprising:
(a) contacting the cellulosic substrate with a f3-glucosidase (BgI), an endoglucanase (EG), a type 2b cellobiohydrolase (CBH2b), a glycoside hydrolase 61(GH61), and the CBH1a variant of claim 1, under conditions in which fermentable sugars are produced; and
(b) contacting the fermentable sugars with a microorganism in a fermentation to produce the end-product.

52. The method of claim 51, wherein prior to step (a), the cellulosic substrate is pretreated to increase its susceptibility to hydrolysis.

53. The method of claim 52, wherein the end-product is an alcohol, an amino acid, an organic acid, a diol, or glycerol.

54-56. (canceled)

57. The method of claims 51, wherein the microorganism is yeast.

58. The method of claims 51, wherein the fermentation is a simultaneous saccharification and fermentation process.

59. The method of claims 51, wherein the saccharification and fermentation steps are consecutive.
